(19) United States
(12) Patent Application Publication
(10) Pub. No.: US 2026/0158242 A1
(43) Pub. Date: Jun. 11, 2026

(54) RECONFIGURABLE STEERABLE CATHETER

(71) Applicant: Technion Research & Development Foundation Limited, Haifa (IL)

(72) Inventors: Shai ELBAZ, Haifa (IL); Amir GAT, Haifa (IL); Nadav ZEMAH, Haifa (IL); Dotan ILSSAR, Haifa (IL)

(73) Assignee: Technion Research & Development Foundation Limited, Haifa (IL)

(21) Appl. No.: 18/708,267

(22) PCT Filed: Nov. 8, 2022
(86) PCT No.: PCT/IL2022/051186

§ 371 Date: May 8, 2024

Related U.S. Application Data
(60) Provisional application No. 63/276,727, filed on Nov. 8, 2021.

(51) Int. Cl. A61M 25/06; A61M 25/00; A61M 25/01

(52) U.S. Cl. CPC A61M 25/0662; A61M 25/0023; A61M 25/0045; A61M 25/0054; A61M 25/0155; A61M 2025/0024; A61M 2025/006; A61M 2025/0063; A61M 2205/0216; A61M 2205/3331; A61M 2205/50

(57) ABSTRACT

The disclosure presents, interalia, a steerable catheter and method for controlling precise tip deflection of a compliant catheter mechanism with plastic and/or elastic deformation capabilities. Tip deflection of the catheter is optionally controlled with a dedicated hydraulic shaping device inserted over a wire in the lumen of the catheter. The hydraulic device can be retracted if necessary. Actuation of the hydraulic device deflects the tip at a desired location along its axis, the process can be repeated in multiple locations thereby shaping the catheter's tip into a desired three-dimensional curve. The deflection may stably maintain a desired shape after the hydraulic device is retracted owing to the multi-stability of the tip structure. In this way a 3D path can be shaped while inside the vasculature or other human anatomy and this 3D path can be maintained and held in position while other trans-catheter devices are passed therealong.

### RELATED APPLICATION

This application claims the benefit of priority of U.S. Provisional Patent Application No. 63/276,727 filed on 8 Nov. 2021, the contents of which are incorporated herein by reference in their entirety.

### FIELD AND BACKGROUND

The present invention, in some embodiments thereof, relates to a configurable tube and, more particularly, but not exclusively, to a configurable guide sheath for intrabody use.

One way of guiding a catheter in the body is to provide a guide sheath and guide a catheter inside the guide sheath.

Additional background art includes US patent publications US20180161547A1, US20140052097A1, US2016249900, US2020/0188635, US20030163154 and US2015352339, US patents U.S. Pat. Nos. 10,456,563, 9,095,374, 10,688,276 and PCT publication WO2008073126 (A1), the disclosures of all of which are incorporated herein by reference.

### SUMMARY

Following is a non-exclusive list including some examples of embodiments of the invention. The invention also includes embodiments which include fewer than all the features in an example and embodiments using features from multiple examples, also if not expressly listed below.
Example 1. A kit comprising:
   a multi-stable guide defining an inner lumen; and
   a shaper sized to fit in said lumen;
   said guide being deformable from a first stable state to a second stable state by said shaper, when said shaper is in said lumen.
Example 2. A kit according to example 1, wherein said shaper is removable.
Example 3. A kit according to any of examples 1-2, wherein said second stable state defines a different bending of said guide than said first stable state.
Example 4. A kit according to any of examples 1-3, wherein said second stable state defines a different extension of said guide than said first stable state.
Example 5. A kit according to any of the preceding examples, wherein said shaper is hydraulic.
Example 6. A kit according to any of the preceding examples, wherein said shaper deforms away from axial symmetry when expanded.
Example 7. A kit according to any of the preceding examples, wherein said shaper has an outer surface configured to frictionally engage an inner surface of said lumen.
Example 8. A kit according to any of the preceding examples, wherein said guide includes an elastic section.
Example 9. A kit according to any of the preceding examples, wherein said guide includes a pliable section.
Example 10. A kit according to any of the preceding examples, wherein said guide includes a multi-stable section arranged to interact with said shaper.
Example 11. A kit according to example 10, wherein said multi-stable section comprises an axial sequence of conical-frusta pairs, each pair defining at least two stable states, each at a different deformation.
Example 12. A kit according to example 11, wherein said section defines at least 30 different stable deformation geometries.
Example 13. A kit according to example 12, wherein said stable deformation geometries include a range of at least 60 degrees of bending between one state and a second state, the bending being measured relative to a longitudinal axis of said sheath.
Example 14. A kit according to any of the preceding examples, wherein said lumen has a cross-sectional area of at least 50% of an area defined by an outer cross-section of said guide.
Example 15. A kit according to any of the preceding examples, wherein said lumen has a cross-sectional area of at least 70% of an area defined by an outer cross-section of said guide.
Example 16. A kit according to any of the preceding examples, wherein said lumen has a cross-sectional area of at least 80% of an area defined by an outer cross-section of said guide.
Example 17. A kit according to any of examples 1-11, wherein said guide defines a second lumen suitable for use as a working channel.
Example 18. A kit according to any of examples 1-11, wherein said shaper defines a lumen extending to and out of a tip of said shaper.
Example 19. A kit according to any of the preceding examples, wherein said guide is sized and of a material suitable for use in the human body.
Example 20. A kit according to any of the preceding examples, wherein said guide has a diameter of less than 30 mm over a length of 1 meter.
Example 21. A kit according to any of the preceding examples, wherein said guide has a diameter of less than 20 mm over a length of 1 meter.
Example 22. A kit according to any of the preceding examples, wherein said guide has a diameter of less than 10 mm over a length of 1 meter.
Example 23. A kit according to any of the preceding examples, wherein said guide is prebent.
Example 24. A kit according to any of the preceding examples, wherein the guide includes a section configured to remain outside the body, when in use.
Example 25. A method of controlling a guide sheath, comprising:
   (a) deforming a portion of said guide sheath using an insert to apply a force;
   (b) manipulating said insert to not apply said force;
   (c) delivering one or both of a tool and a treatment through said deformed guide sheath.
Example 26. A method according to example 25, wherein said deforming comprises expanding said shaper and wherein said manipulating comprises deflating said shaper, at least in part.
Example 27. A method according to example 25, wherein said manipulating comprises removing said shaper and thereby clearing a cross-section of said guide sheath.
Example 28. A method according to example 25 comprising:
   (b1) advancing said sheath after (b); and
   (b2) then repeating (a) and (b) and (b1).
Example 29. A method according to example 28, comprising passing a stented lesion by said advancing.
Example 30. A method according to example 28 or example 29, comprising reaching a distal lesions without guide catheter exchange.
Example 31. A method according to any of examples 25-30, wherein said deforming comprises creating a compound curve in said sheath.
Example 32. A method according to any of examples 25-31, wherein said deforming anchors said sheath in a surrounding body lumen.
Example 33. A method according to example 32, comprising axially shortening part of said bendable section after said anchoring, to advance a proximal part of said sheath.
Example 34. A method according to any of examples 25-33, wherein said delivering comprises directing a guidewire in a desired three-dimensional direction set by said deforming.
Example 35. A method according to any of examples 25-34, comprising cannulating an artery through its ostium and extending said sheath axially beyond the ostium, thereby potentially reducing back-out of said delivered tool.
Example 36. A method according to any of examples 25-35, wherein said deforming provides support for delivery of a device via said sheath by controlling bending and/or axial forces exerted by said sheath.
Example 37. A method according to any of examples 25-36, wherein said deforming provides support for delivery of a device via said sheath by controlling bending and/or axial forces exerted by said sheath.
Example 38. A method according to any of examples 25-37, wherein said deforming comprise controlling one or both of an axial force applied by local extension of said sheath and bending force applied by a bending and/or a three-dimensional curvature of said sheath.
Example 39. A method of controlling a guide sheath, comprising:
   (a) deforming an intra-body portion of a conical-frusta sheath using an insert to apply a force;
   (b) delivering one or both of a tool and a treatment through said deformed guide sheath.
Example 40. A method according to example 39, wherein said deforming is comprised in an act of navigating a tip of said guide sheath to a target location.
Example 41. A method according to example 40, wherein said location is in the heart.
Example 42. A method according to example 41, wherein said location comprises an LV or an

RV outflow region.
Example 43. A method according to example 41, wherein said delivering comprises trans-septally delivering.
Example 44. A method according to any of examples 39-43, wherein said sheath is stably deformed to include at least two curved portions, different in bend radius or direction.
Example 45. A method according to any of examples 39-44, wherein said deforming comprises extending a tip of said guide sheath.
Example 46. A method according to any of examples 39-45, wherein said extending comprises extending a portion distal of a stable bend in said guide sheath.
Example 47. A method according to example 46, wherein said delivering comprises after said delivering, changing a location of said tip to treat an adjacent location.
Example 48. A method according to any of examples 39-47, wherein said deforming includes creating a bend that does not rest against body tissue.
Example 49. A method according to any of examples 39-48, wherein said insert is integrally attached to an inner surface of said sheath.
Example 50. A method according to any of examples 39-49, wherein said deforming comprises selectively deforming by bending decoupled from axial deformation.
Example 51. A method of controlling a guide sheath, comprising:
   (a) selecting a desired bend in said sheath;
   (b) applying a pressure to a deformer associated with said sheath thereby directly setting said bend.
Example 52. A method of controlling a guide sheath, comprising:
   (a) selecting a desired bend and a desired axial extension of a tip of said sheath;
   (b) bending and axially extending a portion of said tip distal to said bend in a decoupled manner whereby said bending does not affect said axial extension and vice versa.
Example 53. A multi-stability guide sheath, comprising:
   a body, comprising:
a lumen;
a bendable section enclosing a part of said lumen,
wherein said bendable section is deformable over a range of positions and wherein each of said positions has an associated resistance to bending and wherein a second plurality of said plurality of positions is characterized by having a deformation-based resistance to bending, wherein said resistance is discrete.
Example 54. A multi-stability guide sheath according to example 53, wherein a plurality of axially separated points along said bendable section each define at least two minima of resistance to bending.
Example 55. A multi-stability guide sheath according to example 54, wherein said points are defined by pairs of conical-frusta elements.
Example 56. A multi-stability guide sheath according to any of examples 53-55, wherein said sheath is stable at said second plurality of said plurality of positions.
Example 57. A multi-stability guide sheath according to any of examples 53-56, wherein said second plurality of said plurality of positions include at least 20 positions.
Example 58. A multi-stability guide sheath according to any of examples 53-55, wherein said body is elastic.
Example 59. A multi-stability guide sheath according to any of examples 53-58, comprising an outer elastic layer overlying a multi-stable under layer.
Example 60. A multi-stability guide sheath according to any of examples 53-59, comprising at least one integral hydraulically expandable chamber which is configured to deform said bendable section, when expanded.
Example 61. A multi-stability guide sheath according to any of examples 53-60, wherein said body defines a guide wire lumen.
Example 62. A multi-stability guide sheath according to any of examples 53-59, comprising at least one removable hydraulically expandable chamber.
Example 63. A multi-stability guide sheath according to example 62, wherein said shaper has an outer surface configured to frictionally and/or geometrically engage a wall of said lumen.
Example 64. A multi-stability guide sheath according to example 62 or example 63, wherein said shaper has a leading edge configured for advancing along said lumen and avoiding geometrically locking therewith.
Example 65. A multi-stability guide sheath according to any of examples 62-64, wherein said shaper has a predefined axially asymmetric shape.
Example 66. A multi-stability guide sheath according to any of examples 53-62, wherein said bendable section has at least one restrictor which non-uniformly limits deformation of said bendable section.
Example 67. A multi-stability guide sheath according to any of examples 53-60, wherein said restrictor defines a compound curve in said bendable section.
Example 68. A multi-stability guide sheath according to any of examples 53-60, wherein said restrictor defines a radially expanding geometry in said bendable section, suitable for anchoring said bendable section in a surrounding body lumen.
Example 69. A multi-stability guide sheath according to any of examples 66-68, wherein said restrictor is elongated.
Example 70. A multi-stability guide sheath according to example 69, wherein said restrictor has a spiral geometry.

As will be appreciated by one skilled in the art, some embodiments of the present invention (e.g., self-advancing robots) may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as guiding a catheter or defining bends in a guide sheath, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1(a)-1(j) show a CFT (conical frusta tip) tool, such as a sheath/catheter and optionally OTW (over the wire) (e.g., double lumen) flexural shaper micro-catheter used for shaping a curve in the CFT, in accordance with some embodiments of the invention;

FIGS. 2(a)-2(m) show a CFT tool and optionally OTW (double lumen) extension shaper micro-catheter, in accordance with some embodiments of the invention;

FIGS. 3(a)-3(m) show a CFT tool and optionally OTW (double lumen) flexural shaper micro-catheter and segmental shaping of a double curve in the CFT, in accordance with some embodiments of the invention;

FIGS. 4(a)-4(n) show a constrained CFT tool and optionally OTW (double lumen) high extension ratio shaper micro-catheter and integral shaping of a complex curve in the CFT, in accordance with some embodiments of the invention;

FIGS. 5(a)-5(i) show a CFT tool and optionally OTW (double lumen) flexural shaper micro-catheter, with intrinsic curvature and/or punctured-sphere-like geometry at the tip, optionally used for shaping a curve in the CFT, in accordance with some embodiments of the invention;

FIGS. 6(a)-6(i) show a CFT tool and optionally OTW (double lumen) flexural shaper micro-catheter, with a fixed antenna-like wire and/or a sphere-like geometry at the tip, optionally used for shaping a curve in the CFT, in accordance with some embodiments of the invention;

FIGS. 7(a)-7(d) show a schematic layout of a catheter tip and top and section views of a constituent multi-stable cell, composed of two elastic conical frusta, in accordance with some embodiments of the invention;

FIGS. 8(a)-8(c) show the theoretical potential energy of a single elastic frustum under zero gauge pressure and a comparison between the theoretical values and the numerically calculated values of the potential energy, in accordance with some embodiments of the invention;

FIGS. 9(a)-9(c) show three projected views describing the dependency of the equilibrium states of a single elastic frustum, on the pressure applied on it directly, in accordance with some embodiments of the invention;

FIGS. 10(a)-10(b) shows a schematic view of a CFT based search and rescue robot, navigational capabilities and gaits, in accordance with some embodiments of the invention;

FIGS. 11(a)-11(p) show a CFT tool and optionally OTW (double lumen) high extension ratio shaper micro-catheter designed to optionally perform intraluminal hydraulic crawling, e.g., when the tip of a CFT guide catheter pulls the shaft distally when actuated by the shaper micro-catheter without the need to push proximally from the hub, in accordance with some embodiments of the invention;

FIGS. 12(a)-12(b) show the idealized and the realistic deformations of the active frustum along (a) the vertical and (b) the horizontal sections of FIG. 8, in accordance with some embodiments of the invention; and

FIG. 13 is a cross-sectional view of an integral device including a sheath having CFT like properties and integrated one or more hydraulic compartments for shaping the sheath, in accordance with some embodiments of the invention.

### DESCRIPTION OF SPECIFIC EMBODIMENTS OF THE INVENTION

The present invention, in some embodiments thereof, relates to a configurable tube and, more particularly, but not exclusively, to a configurable guide sheath or guide catheter for intrabody use.

An aspect of some embodiments of the invention relates to a multi-stable tool, optionally a sheath tool used to deliver other tools, for example, a guide sheath, a guide catheter and/or an elongate tool with a working channel such as an endoscope, optionally for medical use (e.g., sterile packaging). In some embodiments of the invention, the tool (with guide sheath being used as an example) can stably maintain multiple deformation states, for example, in one or two planes, for example, including one, two, three or more bending locations. One or more radio-opaque markers may be provided at deformable locations so that the deformation shape can be better seen under x-ray. In some embodiments of the invention, the deformable part of the tool is a series of conical-frusta elements, and without loss of generality (as the elements may not extend to the actual distal end of the tool and may be located elsewhere along the distal part of the tool) the term CFT (conical frusta tip) is used. It is noted that the designs described herein may also (or instead) be applied to a mid-section and/or a proximal part of a tool.

While the terms guide sheath is used herein, it is noted that a same device can serve both as a guide sheath and as a guide catheter, the functionality optionally depending on the device outer and inner diameters and/or on the target location and paths thereto. In particular, a tool as described herein is optionally steered to a location in the body and other tools may be passed therethrough and/or may be included in the tool itself. Some exemplary tool sizes for medical use include an ID (inner diameter) of 7 F (French)-12 F for a guide sheath and optionally another 1-3 F for the outer diameter (OD), 4 F=7 F for a guide catheter outer diameter and 2.5 F-4 F for a shaper outer diameter, when not expanded. A CFT section may be, for example, between 5 and 500 mm long, for example, between 10 and 200 mm long, for example, between 20 and 150 mm long. A device may include multiple CFT sections separated by non-CFT sections, for example, 2, 3, 4 or more CFT sections. Overall, a device may include, for example, between 2 and 1000 CFT cells, for example, between 10 and 800, for example, between 20 and 100 cells. While the cells may be unconstrained, in some embodiments, between 10% and 90%, for example, between 20% and 70% of the cells are constrained or asymmetric in behavior.

In some embodiments of the invention, the sheath is provided as a kit with an internal shaper, optionally a hydraulically deformable shaper, for example, deformation by expansion, though other shape changes, for example, extending and bending may be provided by deformation in some embodiments. Optionally, the shaper is inserted to a desired bend location in the sheath and expanded to cause a deformation of the sheath. In some embodiments of the invention, the shaper is in the form of an asymmetrically expanding balloon, for example, a balloon which bends as it is inflated and/or a balloon which expands differently at different axial locations and/or a balloon which expands to different amounts at different radial positions (e.g., having a different compliance at different axial parts thereof) and/or a balloon formed of multiple separate expandable compartments which are optionally controlled in parallel or in series.

In some embodiments of the invention, the shaper is designed using calculations, for example, as shown here, to achieve a desired effect on the surrounding CFT. Optionally or additionally, the shaper is capable of a wide range of deformations, some of which are sufficient for CFT manipulation, for example, given suitable inflation pressure.

In some embodiments of the invention, the deformation comprises extension of the sheath. Optionally, the shaper is designed to frictionally engage the inside of the sheath and, optionally, includes two parts which, when expanded, axially translate. In some embodiments of the invention, the shaper includes a surface texture which frictionally engages and/or geometrically interlocks with an inner surface of the CFT. In one example, a plurality of serrations and/or protrusions are provided on one or both of the shaper outer surface and the CFT inner surface. In some embodiments of the invention, a first compartment (or section) of the shaper has a lower resistance to inflation and so inflates first, engaging the CFT inner surface, and allowing the shaper to extend and/or otherwise change shape and modify the shape of the CFT. A potential advantage of such engagement is that forces applied to the CFT can be local, rather than provided (e.g., by pushing) from a remote location such as outside the body. This can have the effect of pulling part of the CFT forward, rather than pushing it form its proximal side. Optionally or additionally, such extension may be useful to allow manipulation of a sheath tip without affecting more proximal deformations.

In some embodiments of the invention, the sheath is deformed, but maintains a stable configuration when deformed. This may be helpful, for example, to allow the sheath to maintain a natural elasticity and spring back after deformation caused by body tissue movement or other forces. In some sense, the geometrical deformation of a CF (conical frusta) element is plastic, as the element switches between two stable states. This type of deformation will be called herein a “bi-stable” deformation. It is noted that once at such a stable state, the element does not apply any force or feel significant strain. Optionally, the CFT material itself can be elastic, in the sense that when its element are not in a stable state, such portion of the CFT can be elastically deformed and spring back when such deforming force is removed.

A potential advantage of a CFT-like structure over a plain elastic wall is that the CFT like structure can provide a stiffer wall. A potential advantage over a wall with an embedded coil or braid is that the CFT has a more structured deformation process. This prevents, for example, uncontrolled deformation as might happen with a coil. Another potential advantage of a CFT like structure is that the CFT structure is stiff, rather than flexible when bent. This stiffness and/or reduction in degrees of freedom may provide a more predictable and stable bending configuration. Also, a CFT structure may be thinner than providing a thick wall that is stiff and/or may require less force to deform.

A potential advantage of a CFT-like structure regarding bending is that as circumferentially related parts in a CFT structure may be coupled, bending is provided by separating points on one side, which causes the other, circumferentially opposite, side to compress. So bending a coil-based flexible catheter may require multiple circumferentially spaced balloons, rather than one directional balloon being sufficient.

In some embodiments of the invention, the CFT segments define a plurality of stable positions (elongation and/or bending), for example, 2, 10, 40, 50, 100 or smaller, intermediate or greater numbers. At such stable positions, the balance of forces applied by conical-frusta sections at various states balance out (e.g., optionally with some friction or plastic deformation forces), so that no net force is applied on the sheath. Further, due to the nature of the conical-frusta design, each conical frusta element is at a local minima, so changing the shape of the CFT would require input of a force to cause the conical-frusta element to leave such minima, providing stability and resistance to deformation (e.g., until sufficient force is applied to further deform the CFT).

In a practical design, the stable states of such CFT may effectively form a continuum of positions where the CFT is stably at rest.

In some embodiments of the invention, the sheath is preformed to be asymmetric, for example, bend in a certain direction when in a resting state, bend more on one (lateral) side of the CFT when a CF element changes state and/or define a non-planar and/or multi-bend shape.

A potential advantage of using a CFT is that force (axial and/or torque) can be applied locally (at the deforming region) rather than proximally.

In some embodiments of the invention, a deformable portion of the sheath (e.g., the CFT) is formed of a sequence (e.g., 4, 10, 20, 30 or intermediate or greater numbers) of conical frusta, optionally circumferentially symmetric and arranged in pairs such that each pair defines together multiple stable states, for example 2. Optionally, sheath deformation comprises deforming one or more such pairs. It is noted that the overall deformation options may be continuous by allowing a continuous set of positions each with a slightly different deformation of a pair of conical-frusta.

In some embodiments of the invention, a different mechanism is used. For example, the sheath may include one or more plastically deformable elements, such as wires, braids, ribbons and/or other structures in its wall, which structures are deformed using an internal shaper.

In some embodiments of the invention, the CFT as a whole (or an axial section thereof) is elastic. Optionally, this functionality is provided by an elastic layer (e.g., elastic wires or coating or sheath). In some embodiments of the invention, this elastic layer is selected to apply an elastic force greater than the force required to change between bi-stable states (e.g., the local minimal force required to keep a frusta cell in an open state). Optionally, this causes the structure to deform momentarily in the bi-stable scheme, under application of force by the shaper, but bounce statically back to its original form. This may be useful for navigation, with the shaper being used to select a bending and/or extending location along the CFT.

It is noted that in this and other embodiments, the CFT may enclose a guidewire, which may be used to maintain a path to a target when the CFT deforms.

In some embodiments of the invention, such a sheath is used to deliver a tool inside the heart or in the GI track. A potential advantage of such use is that a stable and easily traversable path is defined. Another potential advantage of such use is that the deformed sheath possibly does not apply significant additional force on the tissue when a tool passes therethrough and/or in use. Another potential advantage of such use is that the path can be set in stages and one a certain anatomy is traversed, additional manipulation of the sheath or another tool to pass that anatomy is not needed. As with other sheaths, the use of a sheath may protect tissue against inadvertent engagement by a tool passing through the sheath.

An aspect of some embodiments of the invention relates to using hydraulics to set the shape (e.g., bend(s) and/or axial length) of an elongate medical tool, where the shape of the tool is stably maintained also when a hydraulic pressure used to set the shape, is stopped. In some embodiments of the invention, the tool comprises a bendable section with a sequence of conical frusta or a plastically deformable element and an axially adjacent shaping section which is hydraulically deformable, for example expandable, by inflation thereof, thereby stably setting a shape of the deformable section.

In some embodiments of the invention, the shape is set using a hydraulic shaper, which shaper can be removed to allow the medical tool to have a greater available working lumen. Optionally or additionally, the shaper (e.g., when un-inflated) is usable as a micro-catheter over a guidewire.

An aspect of some embodiments of the invention relates to a multi-joint device controllable by an internally inserted shaper which selectively bends one or more of the joints. In some embodiments of the invention, the device is elastically biased so that when the shaper is removed or otherwise stops its shaping action, at least some of the joints bend back to a resting condition thereof. In some embodiments of the invention, the joints are multi-stable structures.

A potential advantage of some embodiments of the invention relates to providing better and/or active control of axial and/or bending forces exerted by a guide sheath (e.g., its bending and/or extension).

A potential advantage of some embodiments of the invention relates to passing narrowing and/or other obstructions, such as stented locations or other implanted devices.

A potential advantage of some embodiments of the invention relates to (more) precise aiming of a guidewire or tool exiting from a guide sheath. Optionally, the better control and stability of a 3D deformation of a sheath is used for such increased precision.

A potential advantage of some embodiments of the invention relates to preventing back-out. Optionally, better and more stable and/or geometrically correct placement and/or shape of a guide sheath in accordance with some embodiments of the invention at an ostium, prevent deformation and/or retrograde movement of the sheath away from the ostium.

A potential advantage of some embodiments of the invention relates to delivery of tools and/or implants, optionally using the sheath as a stable and/or precise aiming platform, with an option for readjustment.

A potential advantage of some embodiments of the invention relates to reaching distal lesions (e.g., 5-10 cm or more past an LM or RCA ostia and/or passing at least two branches/turns from an LM or RCA). Optionally, the methods as described herein are used for maneuvering the sheath so that a guidewire is aimed in a correct direction as the sheath is advanced. In some embodiments of the invention, a potential advantage is that the guide sheath is not exchanged during progress to the lesion.

An aspect of some embodiments of the invention relates to treating the heart using a stably deformable sheath, for example as described herein. In some embodiments of the invention, the sheath is used to set a path to a target lesion, and the sheath is stiff after deployment so that advancing of the tool does not apply force against the body and/or cause damage. Optionally or additionally, the target area is past at least one and optionally two or more different bends in the sheath. In some embodiments of the invention, the tip of the sheath is axially advanced while previous bends stay in place, for example, by axial extension of a CFT portion, for example as described herein. Optionally or additionally, the tip is bend without moving previous bends. A potential advantage is that once navigation of a sheath to a target is complete, at least in a coarse manner, fine tuning may only require application of force at and for the tip.

An aspect of some embodiments of the invention relates to decoupling axial extension and bending of a tubular device. In some embodiments of the invention, axial extension is provided by CFT-type extension of a section of the tip or other part of a catheter or sheath device. As described herein, no mechanical tension or pushing need to be applied along a shaft body of the device. In contrast, standard catheters are bent using pull wires which apply tension all along the shaft and/or axial advancing tends to change the support point of the catheter on a body part, causing a change in bending.

In some embodiments of the invention, bending is provided by CFT-like bending at a location. Desirably, such bending uses locally applied forces which can be delivered by pressured fluid. Preferably, this does not apply tensing, compression or bending forces on any other part of the device, thereby decoupling bending from extending (or contracting).

An aspect of some embodiments of the invention relates to a device with an integral pressured shaping mechanism and a CFT-like multi-stable structure. In some embodiments of the invention, one or more inflation chambers are provided underlying the CFT-like structure. Selective expansion of one or more chamber can provide bending and/or extension, for example, as described herein, noting that the expansion chamber is inherently coupled to the CFT layer by manufacture, so radial expansion to provide friction is not required. Optionally, the expansion chambers are drained after use, so less of the inner volume of the device is taken up. Optionally or additionally, for example in an elastic-return device, the chambers are constrained from radial extension into the device lumen, for example, by an inner non-expanding or radially resilient layer.

An aspect of some embodiments of the invention relates to setting a bending angle of a device using a pressure. In some embodiments of the invention, the pressure is provided using a hydraulic fluid. In some embodiments, the pressure is provided using gas. In some embodiments of the invention, the pressure is provided to a separate expandable shaper inserted within the device. In some embodiments, a pressurizable chamber is integrated into a device. In general, in the disclosure, where a hydraulic actuator is described, it may be replaced by a pneumatic actuator. Generally, pneumatic actuators has the potential advantage of being more elastic, while hydraulic actuators have the potential advantage of being more accurate.

A potential advantage of using pressure is the option of avoiding applying force to parts of the device other than the part being intentionally deformed. This allows one part of the device to be deformed while other parts stay stable. In particular, no retracting forces, pushing forces, rotational torque and/or length-changing force are present in some embodiments.

An aspect of some embodiments of the invention relates to a catheter with a bend radius less than 150% (or less than 120% or less than 100%) of the diameter of the catheter. In one example, the bend radius is between 3 and 5 mm, or less, for example for a catheter diameter of between 3-5 mm. In some embodiments of the invention, such radius of bending, for a rigid and/or elastic catheter are provided using CFT-like structures which optionally provide support to avoid crimping of any catheter lumen.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### DESCRIPTION OF SOME EMBODIMENTS OF THE INVENTION

One proposed steerable catheter mechanism relies on a combination of component devices: A conical frusta tip (abbreviated CFT) structure which forms the distal portion of the steerable catheter and the hydraulic device which is inserted into the lumen of the steerable catheter (e.g., either in a monorail formation or over a guidewire-OTW). While each device in separate: the conical frusta tip or the hydraulic device (also referred to as a “shaper”) are optionally relatively simple mechanisms, it is the combination of the two that can potentially provide precision steering with a variety of shape changing capabilities as described herein. The catheter's shaft is optionally wire-free; it does not require any special features or specifications as per some embodiments of the invention (e.g., threading of pull-wires or other embedded mechanisms) and thus can potentially be designed for optimal torque transfer. The resulting wall thickness of the catheter's distal portion can potentially be significantly smaller than conventional steerable catheters or sheaths which do incorporate pull-wires. Furthermore, the control of the tip is optionally independent of the shaft structure, length or material. In some embodiments of the invention, control of the tip is achieved via pressurization of the in-lumen retractable hydraulic device connected to the hub of the catheter.

### An Exemplary Conical Frusta Tip

In some embodiments of the invention, the conical frusta tip comprises a portion, optionally distal, of the catheter that undergoes deflection, in some embodiments of the invention. The design parameters of the conical frusta (e.g., geometrical definitions such as conical element angles and heights, material definitions such as wall thickness, and rigidity and additional parameters as describe herein) and the resulting functionality of the catheter are optionally linked together as described herein.

In conventional non-steerable catheters and in some steerable catheters the distal portion of the catheter may be pre-shaped in order to provide a certain functionality (e.g. support against an artery). In general, in some embodiments of the current invention the distal portion of the catheter is not pre-shaped, although it is possible to pre-shape it. The conical frusta tip can undergo dynamic shaping while inside the vasculature as it is actuated by the hydraulic shaper catheter (e.g., see description of drawings above).

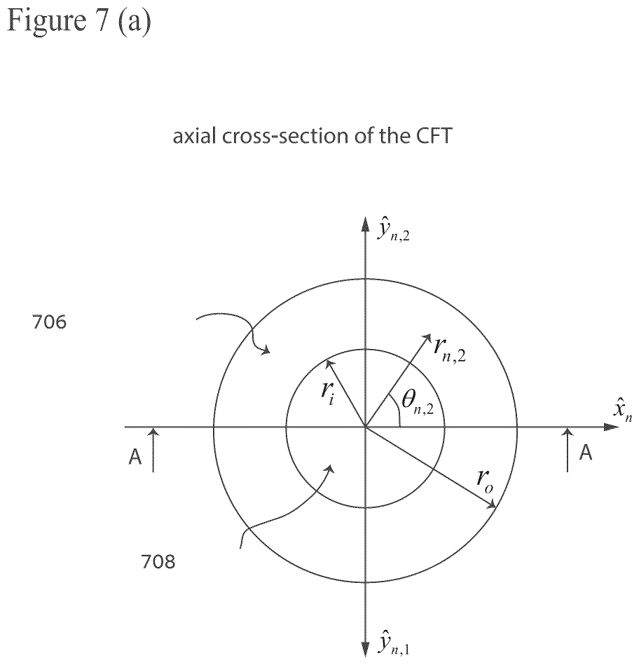

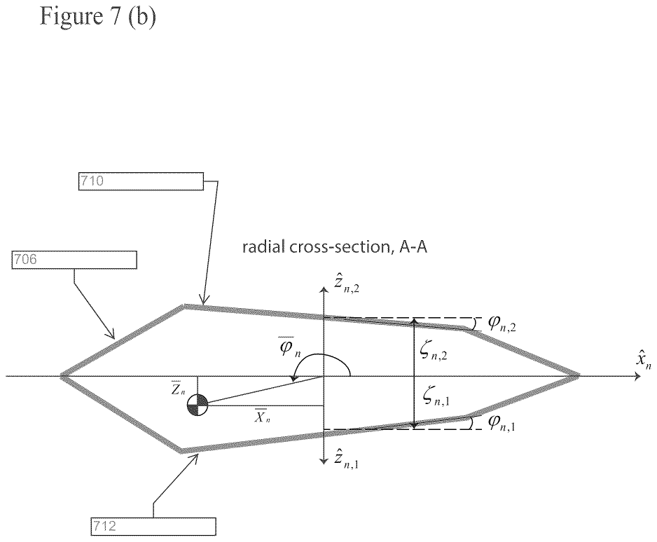

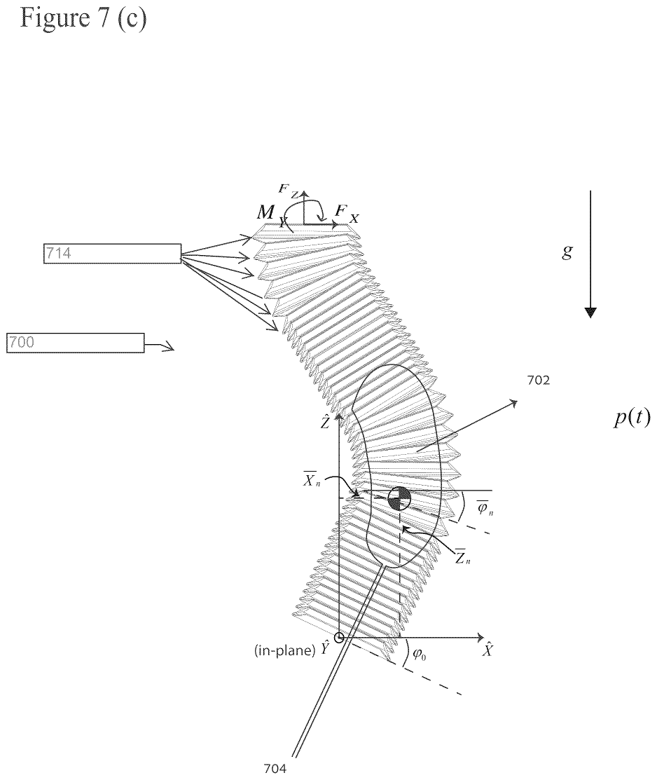

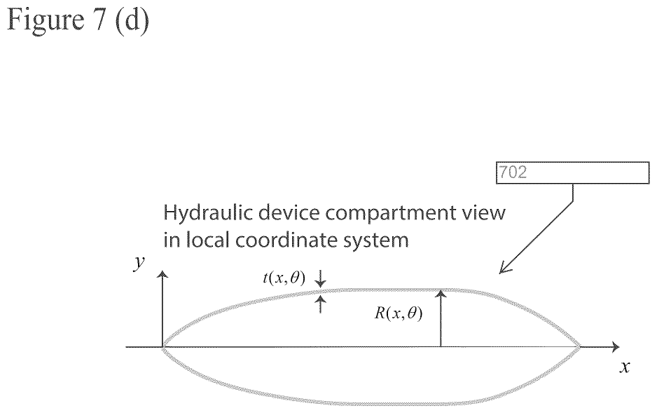

In this document, shaping is defined as either permanent or momentary change of local two- or three-dimensional orientation of the axis of the tip segment along any point of its axis. For example, both curvature and deflection of the axis can be controlled in three-dimensional space to form a desired three-dimensional curve. Momentary, pressure actuation dependent shaping of the CFT, can be achieved by incorporating an elastically deformable lining into the CFT, for example, either on the inner wall, or on the exterior wall. Alternatively, momentary, pressure dependent, shaping of the CFT can be achieved, for example, with the CFT design shown in FIG. 7 and a dedicated shaping device with an elastic spring layer on the concave arc of a single lumen shaper.

The pre-actuated axial length of the conical frusta tip is denoted by lₜ. In some embodiments of the invention, the tip can undergo axial extension in the range of 1:1 to 1:lᵣ, where lᵣ denotes the extension ratio. While the analysis will regard the tip length as arbitrary, in general it can vary between applications. For example, for a 110 cm (usable length) long steerable catheter, the conical frusta tip can comprise 10-20 cm of this length. Optionally, the length of conical-frusta parts of such a tube may be, for example, 10%, 20%, 30%, 40%, 50% or greater, smaller or intermediate percentages of the length of the tube.

FIG. 7 (c) shows a schematic layout of a catheter's tip 700 and in-lumen hydraulic device 702, with a hydraulic pressure source 704.

FIGS. 7 (a) and 7(b) show top and section views of a constituent multi-stable cell, composed of two elastic conical frusta (710, 712), with FIG. 7(a) showing a top frustum face 706 and a CFT lumen 708 in accordance with some exemplary embodiments of the invention. FIG. 7(d) shows a section view of hydraulic device 702 with corresponding static thickness and radial functions.

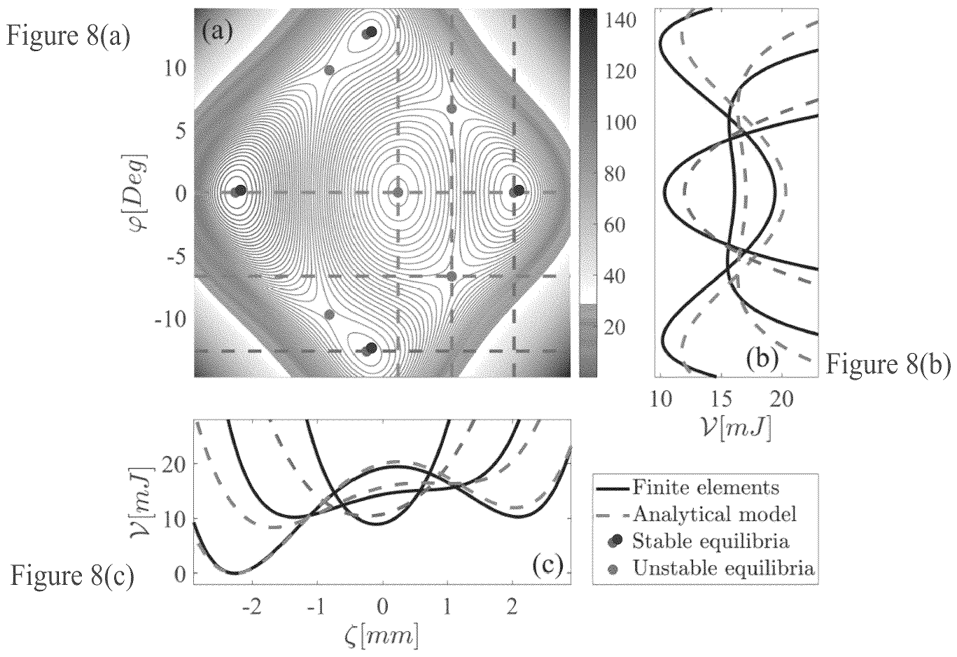

The conical frusta tip shown in FIG. 7, is composed of a serial interconnection of sets of two elastic frus-a—a diverging frustum 710 and a converging frustum 712 oppositely aligned along the axis. In some embodiments of the invention, each set, or sub-structure, has both uniaxial and anti-symmetric degrees of freedom. Under appropriate conditions each set has four stable equilibrium states (e.g., see FIG. 8). This local multi-stability potentially provides the elements the ability of staying stable in a large number of complex equilibrium states, which can be beneficial for reconfiguring the shape of the catheter tip. It is noted that while the overall effect is multi-stable deformability of the shape (which appears to be plastic deformation), the actual structure may be elastically deformable.

As illustrated in FIG. 7, catheter (or guide sheath) tip 700 is optionally described as a serial interconnection of N cells 714, each consisting of two elastic frusta. The cells optionally interface a pressurizeable device 702, with pressure denoted p as seen in FIG. 7 (c), (the hydraulic device) located in the lumen 708 of the tip segment which is controlled externally. We consider a two-dimensional description where the deflection of the tip and its constituent building blocks are bounded to the \hat{X}-\hat{Z} plane of the global coordinate system whose origin is located at the middle of the tip's base where it is connected to the distal portion of the shaft of the catheter, see FIG. 7 (c). The deformation of each n^{th} multi-stable cell 714 of the tip is formulated in terms of ζ_{n,1} and φ_{n,1} denoting the axisymmetric and antisymmetric DOFs (degrees of freedom) of the frustum closer to the base of the tip, as well as ζ_{n,2} and φ_{n,2} describing the corresponding DOFs of the frustum that is farther from the base of the tip.

### Exemplary Coordinates of Motion of the Tip Structure

In some embodiments of the invention, the horizontal and vertical coordinates of the n^{th} multi-stable cell's center of mass with respect to the global coordinate system are denoted as:

$${\overline{X}}_{n} = {\sum }_{j = 1}^{n - 1} ( {ζ}_{j , 1} + {ζ}_{j , 2} ) sin {\overline{Φ}}_{j} + ( {ζ}_{n , 1} + {\overline{z}}_{n} ) sin {\overline{Φ}}_{n} + {\overline{x}}_{n} cos {\overline{Φ}}_{n} \qquad ( 1 )$$

$${\overline{Z}}_{n} = {\sum }_{j = 1}^{n - 1} ( {ζ}_{j , 1} + {ζ}_{j , 2} ) cos {\overline{Φ}}_{j} + ( {ζ}_{n , 1} + {\overline{z}}_{n} ) cos {\overline{Φ}}_{n} - {\overline{x}}_{n} sin {\overline{Φ}}_{n} \qquad ( 2 )$$

The orientation of the cell's middle surface around the Ŷ axis is denoted as:

$${\overline{Φ}}_{n} = {φ}_{0} + {\sum }_{j = 1}^{n - 1} ( {φ}_{j , 1} + {φ}_{j , 2} ) + {φ}_{n , 1} \qquad ( 3 )$$

where φ₀ is the inclination angle of the tip's base, relative to the horizon. The above equations are optionally supplemented by the translational and angular coordinates of the n^{th} multi-stable cell's center of mass, with respect to the local coordinate system of its constituent frustum, which is farther from the tip's base (see FIG. 7 (b)):

$${\overline{X}}_{n} = - \frac{{r}_{o} {r}_{i} ( {φ}_{n , 1} + {φ}_{n , 2} )}{4 ( {ζ}_{n , 1} + {ζ}_{n , 2} )} \qquad ( 4 )$$

$${\overline{z}}_{n} = \frac{2 ( {r}_{o}^{2} + 3 {r}_{i}^{2} + 2 {r}_{o} {r}_{i} ) ( {ζ}_{n , 2}^{2} - {ζ}_{n , 1}^{2} ) + {r}_{o} {r}_{i}^{2} ( {r}_{o} + 2 {r}_{i} ) ( {φ}_{n , 2}^{2} - {φ}_{n , 1}^{2} )}{8 ( {r}_{o}^{2} + {r}_{i}^{2} + {r}_{o} {r}_{i} ) ( {ζ}_{n , 1} + {ζ}_{n , 2} )} \qquad ( 5 )$$

$${\overline{φ}}_{n} = a tan 2 ( {\overline{z}}_{n} , {\overline{x}}_{n} ) \qquad ( 6 )$$

The horizontal, vertical and rotational DOF of the tips distal outlet may be denoted as:

$${X}_{tip} = {\sum }_{j = 1}^{N} ( {ζ}_{j , 1} + {ζ}_{j , 2} ) sin {\overline{Φ}}_{j} \qquad ( 7 )$$

$${Z}_{tip} = {\sum }_{j = 1}^{N} ( {ζ}_{j , 1} + {ζ}_{j , 2} ) cos {\overline{Φ}}_{j} \qquad ( 8 )$$

$${Φ}_{tip} = n + {\sum }_{j = 1}^{N} ( {φ}_{j , 1} + {φ}_{j , 2} ) \qquad ( 9 )$$

### Exemplary Contribution of Nonconservative Forces Applied to the System

To consider the effect of the nonconservative forces applied to the system, the virtual work may be calculated. These forces may include the uniform pressure applied to the catheter's closed end (for example by the present arterial blood or by the hydraulic device 702) assuming its terminal radius equals to rᵢ, and/or the local horizontal and vertical forces F_{X} and F_{z}, and/or the bending moment M_{γ} applied to this end (see FIG. 7 (c)). These forces can also be applied by the artery (or other surrounding lumen or nearby tissue or other objects) in case of arterial anchoring and supports. Moreover, the gravitational forces applied by the fluid entrapped inside the CFTs cells is also optionally taken into account. Considering all the effects mentioned above, the total virtual work may be given by:

$$δ𝒲 = - ρ g {\sum }_{n = 1}^{N} {V}_{n} δ {\overline{Z}}_{n} + ( {F}_{X} + π {r}_{i}^{2} p sin {\overline{Φ}}_{t i p} ) δ {\overline{X}}_{t i p} + ( {F}_{Z} + π {r}_{i}^{2} p cos {\overline{Φ}}_{t i p} ) δ {\overline{Z}}_{t i p} + {M}_{Y} δ {\overline{Φ}}_{tip} ,$$

where g is the gravitational acceleration, δ● is a variation in ●.

### Exemplary Closed Form Expressions for the Axial Force and Bending Moment Applied Externally to a Single Frustum

In some embodiments of the invention, the force and moment applied by the interfacing hydraulic device 702 or neighboring frusta as experienced by a single frustum is represented by:

$$F \approx 4 {C}_{1} ζ ( 2 {ζ}^{2} - 2 {ζ}_{0}^{2} + 3 {r}_{i}^{2} {φ}^{2} ) + 2 {C}_{2} ( ζ - {ζ}_{0} ) + {C}_{3} p , \qquad ( 10 )$$

$$M \approx {r}^{2} φ [ {C}_{1} ( 12 {ζ}^{2} - 4 {ζ}_{0}^{2} + 3 {r}_{i}^{2} {φ}^{2} ) + {C}_{2} ] , \qquad ( 11 )$$

Here, ζ₀≈(rₒ−rᵢ)ψ₀ is the load-free deflection of the frustum and p signifies hydraulic compartment (e.g., of shaper) quasi-steady time-dependent actuation pressure. The constant C₁, C₂, C₃ may be given by:

$${C}_{1} = π Eh [ ( {r}_{o} + {r}_{i} ) / 2 - ( {r}_{o} - {r}_{i} ) / ln ( {r}_{o} / {r}_{i} ) ] / 8 {( {r}_{o} - {r}_{i} )}^{3} \qquad ( 12 )$$

$${C}_{2} = π {Eh}^{3} ln ( {r}_{o} / {r}_{i} ) / 12 {( {r}_{o} - {r}_{i} )}^{2} \qquad ( 13 )$$

$${C}_{3} = π ( {r}_{o}^{2} - 2 {r}_{i}^{2} + {r}_{o} {r}_{i} ) / 3 \qquad ( 14 )$$

### Exemplary Solving for the Final Shape of the Catheter Under Hydraulic Actuation

In some embodiments of the invention, combining the integrations of equations (10) and (11) with respect to ζ and φ, and reinstating the general subscript n,k (cell and frustum respectively) yields the potential energy of a general frustum, given by:

$${V}_{n , k} \approx 2 {C}_{1} {ζ}_{n , k}^{4} + ( {C}_{2} - 4 {C}_{1} {ζ}_{0 , k}^{2} ) {ζ}_{n , k}^{2} - ( 2 {C}_{2} {ζ}_{0 , k} + {C}_{3} p ) {ζ}_{n , k} + \frac{3 {C}_{1} {r}_{i}^{4}}{4} {φ}_{n , k}^{4} + \frac{{C}_{2} - 4 {C}_{1} {ζ}_{0 , k}^{2}}{2} {r}_{i}^{2} {φ}_{n , k}^{2} + 6 {C}_{1} {r}_{i}^{2} {ζ}_{n , k}^{2} {φ}_{n , k}^{2} + Const . \qquad ( 15 )$$

Here it is assumed that all odd frusta are identical as are all even frusta (this is not required in some embodiments), and ζ_{0,k} denotes the load-free deflection of the k^{th} frusta in each multi-stable cell.

Optionally, the potential energy of the hydraulic compartment contributed by the n^{th} cell is given by

$${V}_{H D , n} = π {Er}_{n o m} \frac{{h}_{0} {ℓ}_{0}}{{ℓ}_{0} + {ζ}_{n , 1} + {ζ}_{n , 2} - ( {ζ}_{0 , 1} + {ζ}_{0 , 2} )} ( {ζ}_{n , 1} + {ζ}_{n , 2} ) ( \frac{3 {r}_{i} {φ}_{n , 2}^{2} + 2 {( {ζ}_{n , 1} + {ζ}_{n , 2} )}^{2}}{2 {ℓ}_{0}^{2}} ) \qquad ( 16 )$$

where, [symbol] ₀=ζ_{0,1}+ζ_{0,2}, h₀ is nominal cell height and rₙₒₘ is nominal cell radius.

The total energy of the catheter's CFT portion is optionally given by summing the energy over all the cells of the tip and adding the energy of the interfacing shaper catheter as follows:

$${V}_{t o t a l} = {\sum }_{n = 1}^{N} \{ {\sum }_{k = 1}^{2} {V}_{n , k}^{f r u s t a} + π E ( \frac{{h}_{0} {ℓ}_{0}}{{ℓ}_{0} + {ζ}_{n , 1} + {ζ}_{n , 2} - ( {ζ}_{0 , 1} + {ζ}_{0 , 2} )} ) {r}_{n o m} ( {ζ}_{n , 1} + {ζ}_{n , 2} ) ( \frac{3 {r}_{i} {φ}_{n , 2}^{2} + 2 {( {ζ}_{n , 1} + {ζ}_{n , 2} )}^{2}}{2 {ℓ}_{0}^{2}} ) \} \qquad ( 17 )$$

In one manner of solving, one applies Hamilton's principle on this sum, e.g., in the current equation system differentiating the total energy sum (potential and virtual work) according to each degree of freedom and equating to zero, which yields a system of 4N ordinary differential equations, governing the dynamics of the system under investigation. Their solution can yield the controlled deflection of the CFT under hydraulic actuation of the shaper. Furthermore, equations (10), (11) form a set of governing equations of motion which can be solved for the local DOFs (degrees of freedom) of each frustum: ζ, φ. In this way, external forces applied from the artery, for example in the case of arterial supports, can optionally be incorporated into the solution of the spatial motion of the CFT catheter. Equations (10), (11) have another potentially significant importance in that they describe the static tip behavior once the hydraulic device has been retracted. They can provide design criteria as to the local forces and moments supported by the tip structure within the artery. For example, if the catheter's tip is positioned in a support formation against the artery (or a heart wall or other dynamic structure), the design criteria can ensure that the tip structure will not change shape due to this support.

### Exemplary Solutions to the Governing Equations and Analysis

Examining the elastic nature of a single frustum while it is not subjected to hydraulic device pressure (or shaping), based on the theoretical formulation, and utilizing a finite element analysis, one can deduce the fundamental multi-stable behavior of the CFT section.

FIG. 8 shows the strain energy function of a single active frustum based on eq. (15) and the parameters computed in the calibration process, in accordance with some embodiments of the invention. This figure also presents the frustum's equilibrium states achieved by nulling eqs. (10) and (11), where the stability of each state is classified based on the eigenvalues of the Jacobian of these equations at the examined state.

FIG. 8 further shows several sections of the strain energy function, where either the axial or the tilting DOF is constant. Finally, the corresponding sections achieved from a finite element scheme devised using the COMSOL Multiphysics software are also displayed for validation. In this scheme, the frustum is discretized by second order rectangular shell elements, distributed uniformly such that there are 15 elements in the radial direction and 100 elements along the circumference. Numerous stationary simulations utilizing this scheme were performed, where the axial displacement of the frustum's small base is determined, whereas its large base is bounded to a plane in a way that allows rotations. The strain energy in each state was computed by a built-in function.

FIG. 8 shows a very good agreement between the theoretical and the numerically obtained results in moderately small values of the DOFs, whereas significant values of ζ and φ appear to lead to weaker correlation as they result in large values of the base angle ψ which is assumed to be small for the particular model derivation. Nevertheless, the range of validity seems adequate to capture most reasonable deformations of the frustum and illustrates that both forward and reverse problems can be solved—CFT geometry based on force and force needed to achieve a CFT geometry.

Next, FIG. 8 shows that when the frustum is not subjected to pressure it has nine equilibrium states, four of which are stable and correspond to snap-up, snap-down and two anti-symmetrical partial-snap states. These stable states provide a non-pressurized (e.g., with no internal hydraulic or other pressure) CFT the capability of staying stable in a large number (e.g., due to the large number of frustums) of equilibrium states that include local folding, deploying, and bending. In the realistic case where the thickness of the frustum is not infinitely small, the strain energy function and its equilibrium states are not symmetrical around ζ=0.

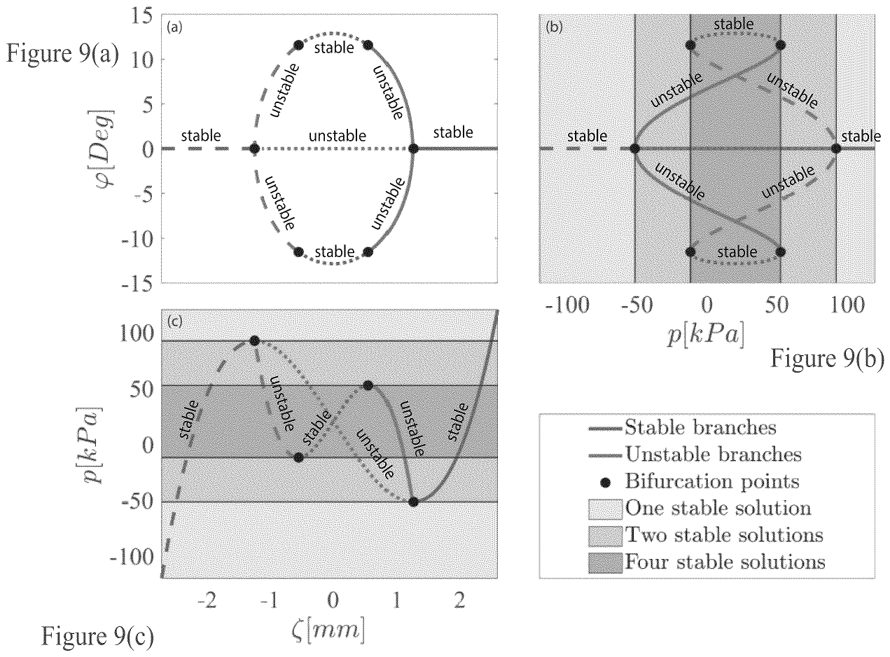

To complete a picture of a frustum's exemplary static behavior, FIG. 9 shows the analytically obtained equilibrium states of the active frustum achieved in conditions where different external hydraulic device pressures are applied to it directly. This figure shows four bifurcation pressure values, separating five regions with different number of stable states. The first region corresponds to small absolute pressure values thus is described qualitatively by FIG. 8, meaning that in this region the frustum has four stable states. Conversely, in high positive pressure values the frustum can stay stable only in a snap-up state, and similarly in high negative pressure values the only equilibrium state is snap-down. The two supplementary regions are achieved in moderate pressure values (positive or negative). In these regions the frustum has two stable equilibria, corresponding to snap-up and snap-down states. Accordingly and in agreement with FIG. 8, relatively small pressure variations inside a CFT (or interfacing hydraulic compartment) will cause post-buckling from snap-up or snap-down state to a partial-snap as it is energetically preferable. However, in higher pressure values each frustum will switch immediately between snap-up and snap-down states. One potential benefit is that a bent configuration can be straightened to an axial extension configuration by increasing pressure. This also allows sequential deformations of a same part of a CFT, as well as sequential deformations of different parts of a CFT. The last analysis executed which deals with the system's elastic properties examines the assumption that all odd frusta can be considered rigid. This analysis utilizes a finite-element scheme established in COMSOL Multiphysics, describing a multistable cell composed of two interconnected frusta with identical thicknesses, whose unstressed axisymmetric deflections are ζ0,1 and ζ0,2.

In agreement with the model's assumptions the bases of both frusta are allowed to rotate freely around the tangential direction where the large bases of both frusta are forced to have equal translational motion, the axial displacement of the upper frustum's small base is dictated, and the small base of the lower frustum is pinned to a plane. Similarly to the scheme discussed above, each frustum is discretized by second order rectangular shell elements, dividing it to 15 radial and 100 tangential evenly distributed segments.

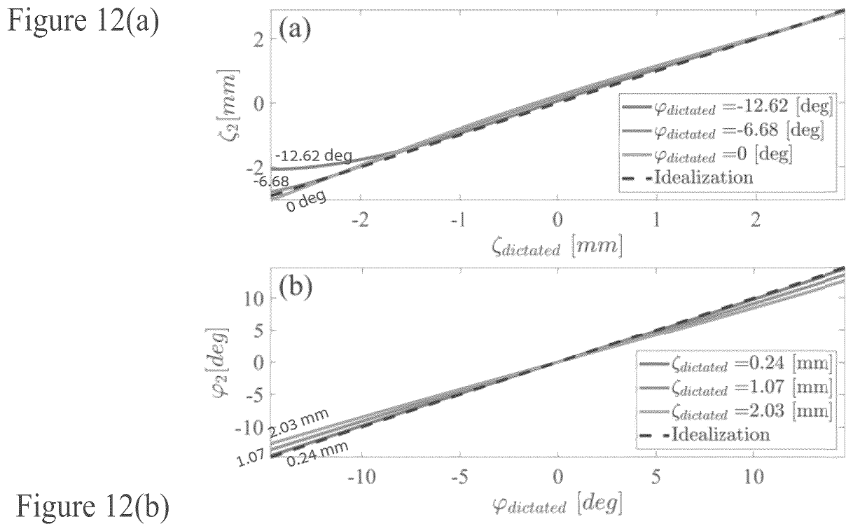

Based on this scheme, FIG. 12 compares the idealized deformations of the cell represented only by those of the upper frustum, and the deformation of this frustum in a more realistic case where the lower one is not considered rigid. This figure shows that in moderate values of ζ and φ which suit most practical deformations, the deviations between the realistic and the idealized cases are small, implying that the model simplification which truncates half of it's DOFs is indeed applicable.

To summarize, this energetic stability map, obtained from numerical simulation, shows both stable equilibria points and unstable equilibria points. In FIG. 8 the extension DOF is zeta, and curvature DOF is phi. At zero phi there are two stable axial extension points meaning the cell can be energetically stable when collapsed and when opened. Alternatively, at zero zeta there are two stable curvature points, since the analysis is two-dimensional, the structure can flex to either side (e.g., without loss of generality, either side means in any azimuthal direction of three dimensions).

### Exemplary Hydraulic Shaper Catheter

In some embodiments of the invention, the Hydraulic actuation (e.g., shaper) is generated via a dedicated in-lumen transcatheter (operating in the lumen of the conical frusta tipped catheter) device. The hydraulic device's shaft structure can be implemented using embodiments which are similar to the shaft portion of PTCA Balloon angioplasty catheters, e.g., OTW-over the wire-a double lumen shaft with a guidewire lumen and inflation lumen or monorail/Rapid exchange-a shaft with a single central inflation lumen and distal guidewire entry port, or a shaft with a single central inflation lumen that no wire is incorporated with it. The tip of the shaper catheter optionally comprises an elastically deformable compartment which assumes a predesigned shape when pressurized (e.g., such the shape is optionally a function of its elastic and geometrical properties). Such compartment may have axially and/or tangentially varying radius and/or wall-thickness, Young's modulus, Poisson's ratio and/or other parameters. Optionally, the general shape conforms to the cylindrical shape of the tip of the shaper catheter 702 as seen in FIG. 7.

The properties of the elastically deformable compartment (e.g., radial cross-section, arc sector, length and/or elastic material constants) optionally determine its functionality while pressurized inside the lumen of the CFT. The elastically deformable compartment is optionally made of, but not limited to, compliant hyperelastic material. Optionally, a stress-strain curve of such material is designed according to its desired functionality (e.g., with appropriate choice of specific material). In some embodiments of the invention, the design process is carried out via simulation in closed form or numerical solution of the governing equations of the structure, for example, as described above. It is noted that for any given desired deflection of the CFT, there are often several different embodiments of hydraulic shaper tips which such generate desired deflection of the CFT. It is also noted that the shaper tip may be predesigned to expand and/or apply forces in a manner which is non-uniform axially and/or circumferentially. Further, the tip may be designed to apply different forces radially, for example, one part may be compliant and one part non-compliant. Increasing pressure past a certain point will further expand the compliant section, potentially further deforming a surrounding CFT, but not substantially affect the non-compliant section.

### Exemplary Navigation of the Vasculature/Heart Chambers Using an OTW Hydraulic Shaper Micro-Catheter and a Conical Frusta Catheter—CFT Tipped Catheter.

The set of hydraulic shapers as described herein can generate a wide range of deflections of the CFT. Optionally, one or both of two types of pressure signals are provided by such a shaper: constant—uniform pressures (such as used for example in PTCA balloon devices) and time dependent pressure signals. Time dependent pressure signals can create a corresponding desired transient response of the CFT which may be optionally used to navigate the catheter in the vasculature (e.g., if the sheath/catheter is not deformed to a new stable configuration). The mechanism by which the shaper generates either elastic deflections or plastic (reconfigurable) deflections of the CFT is a measured combination between local axial elastic strain, generated in the shaper's elastic compartment, and the local tangential (circumferential) strain which enforces contact force along the CFT, for example, as described herein with respect to an elastic sheath covering the CFT.

### SOME EXEMPLARY MODES OF OPERATION

There are several embodiments of hydraulic actuation. One of the main approaches, viable, for example, for outer sheaths or catheter diameters larger than 4 Fr, for example, is an independent hydraulic shaper catheter: the shaper and CFT are aligned, and twisting of the shaper changes the directionality of the curve without the need to twist the CFT sheath. The following table summarizes some exemplary shaper embodiments:

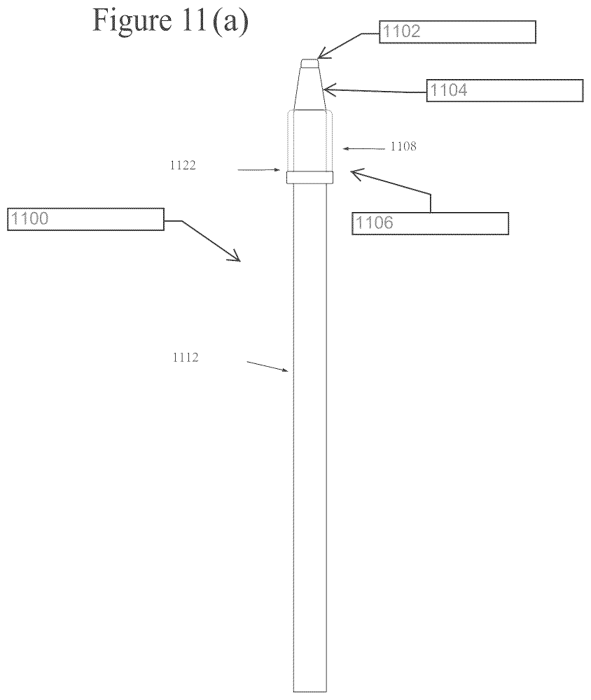

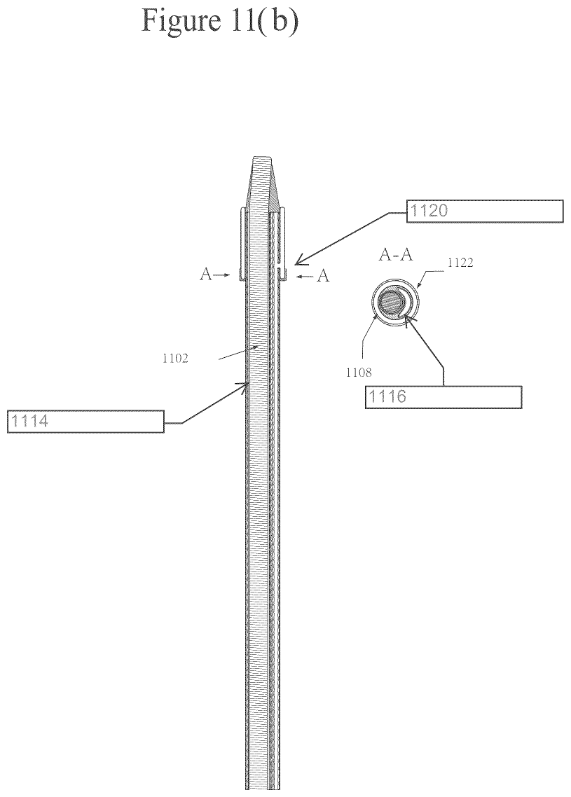

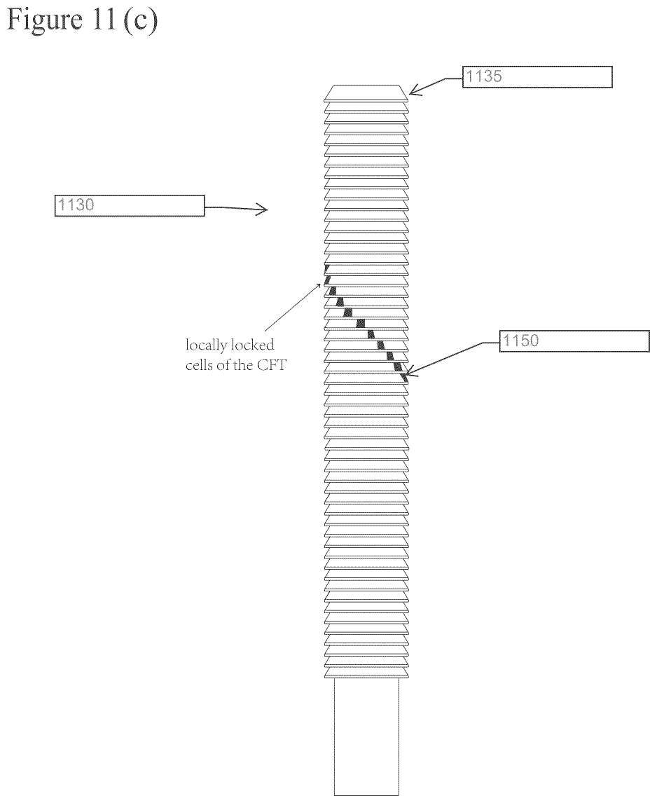

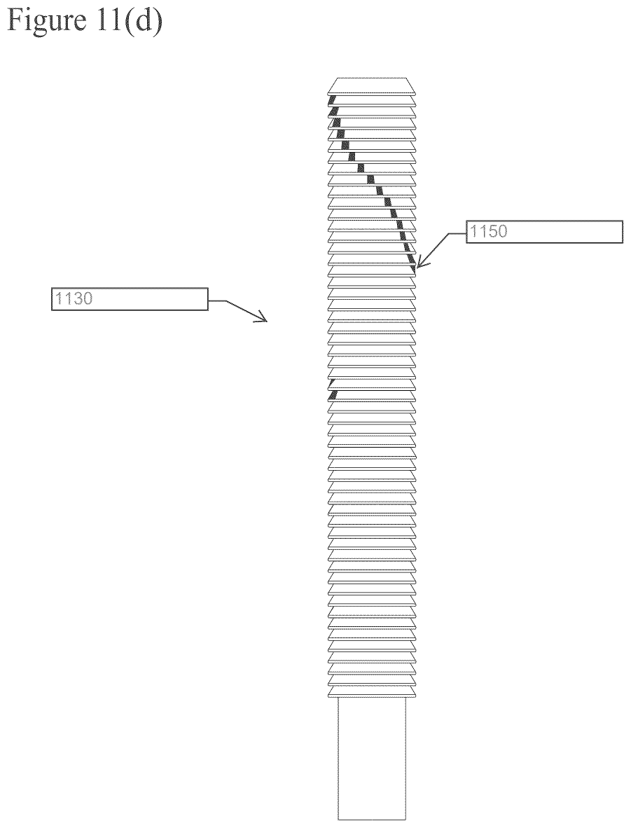

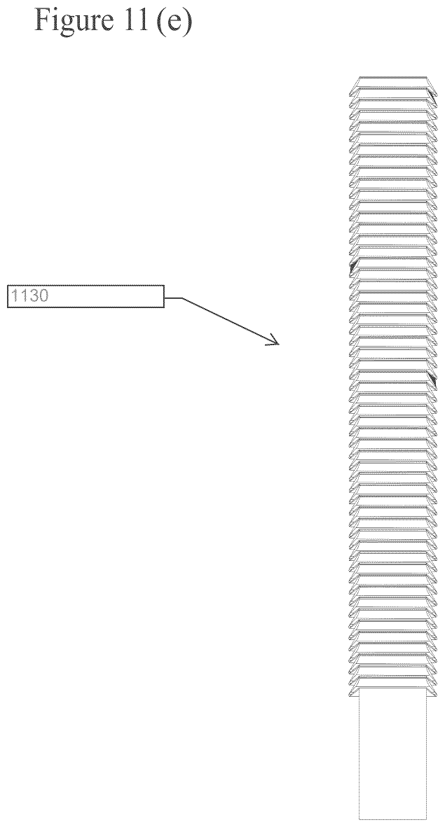

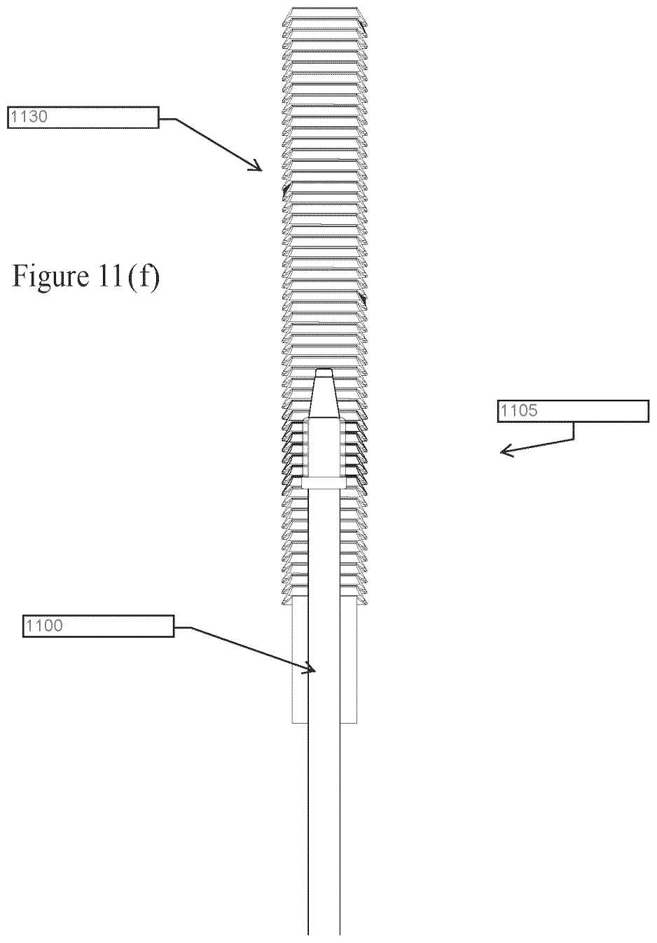

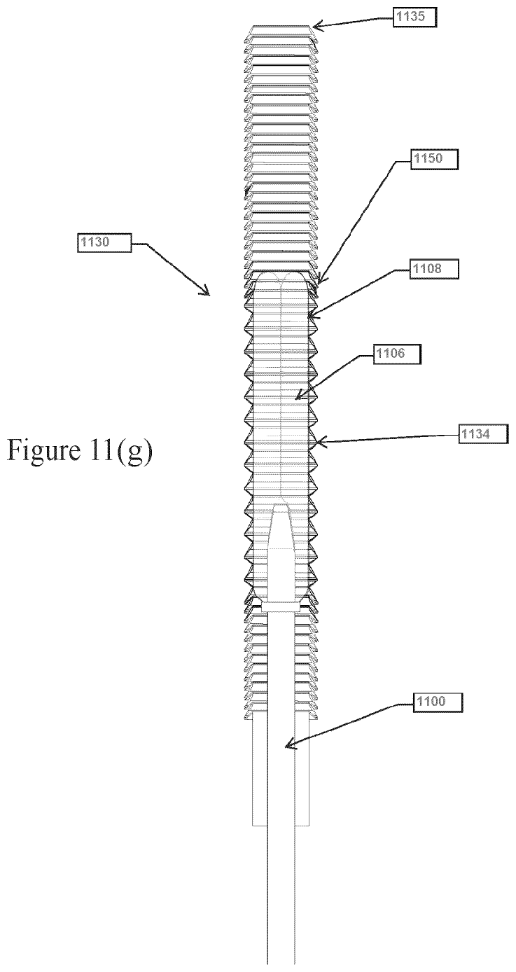

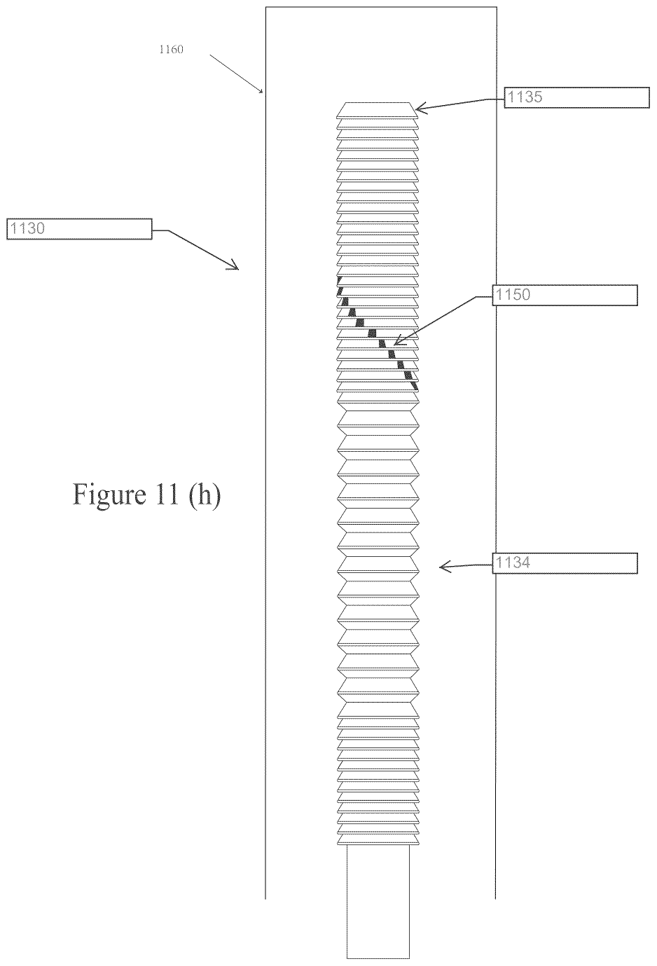

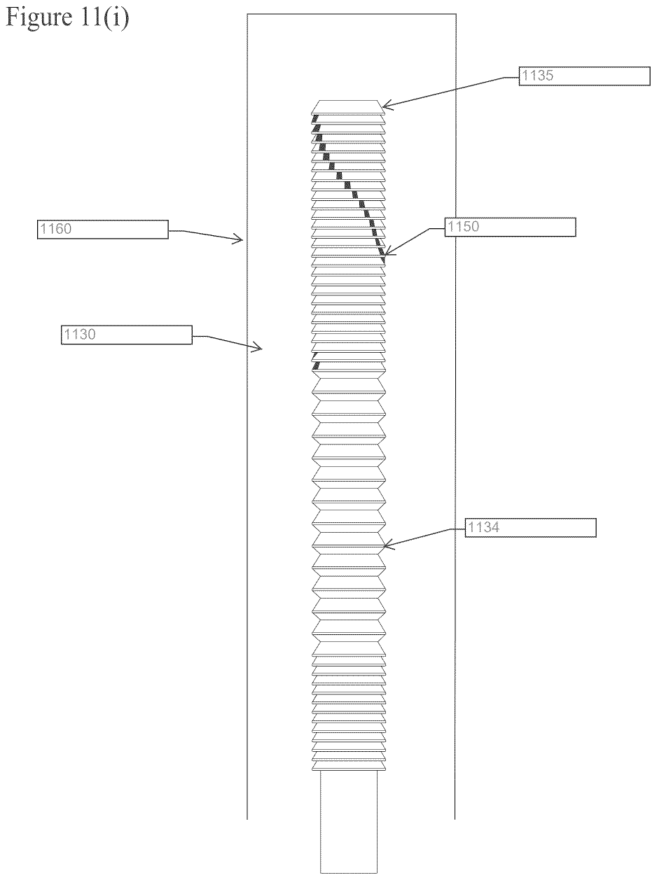

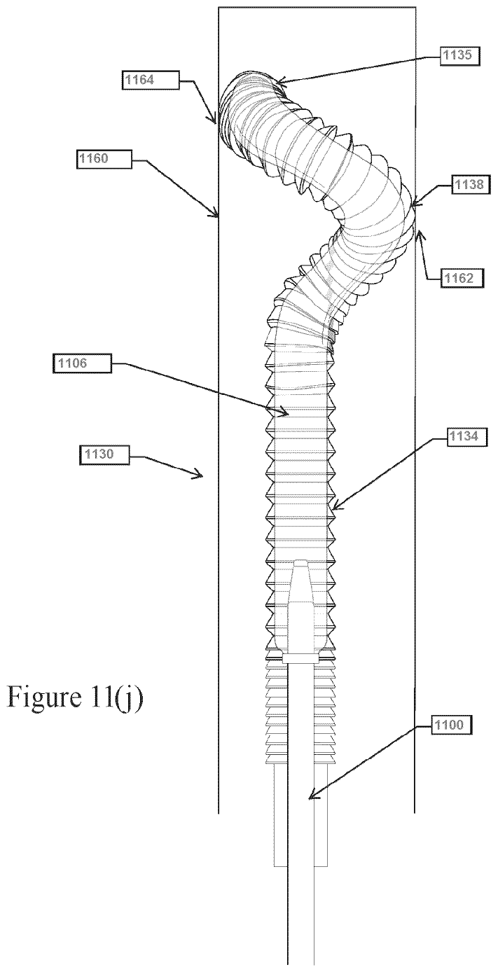

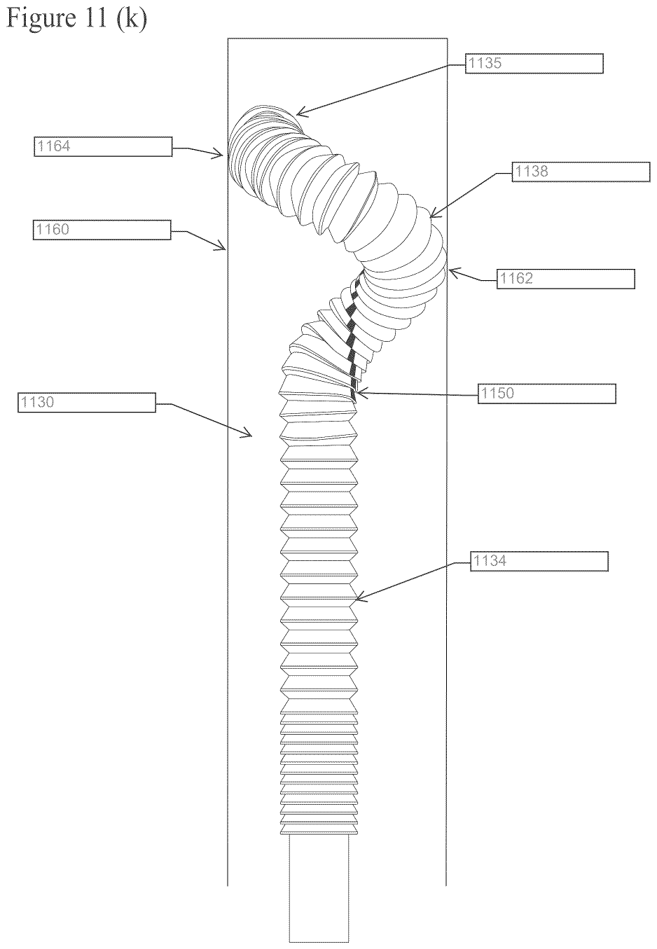

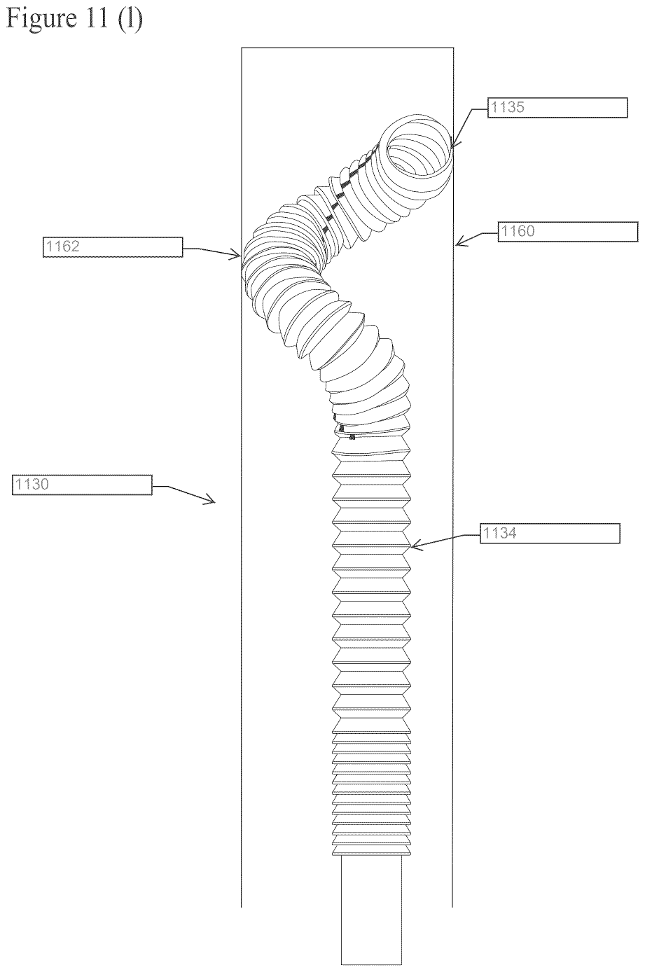

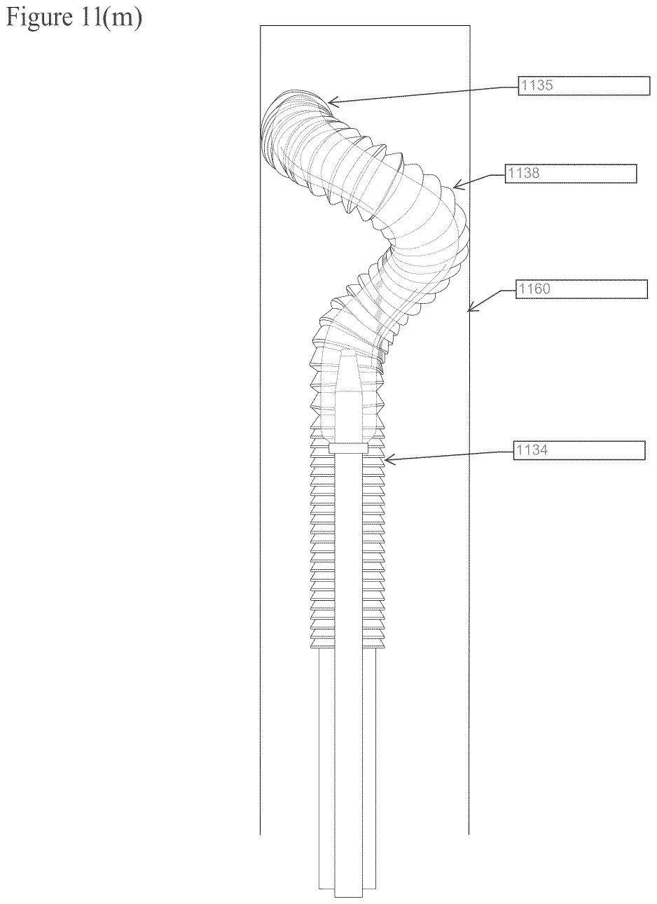

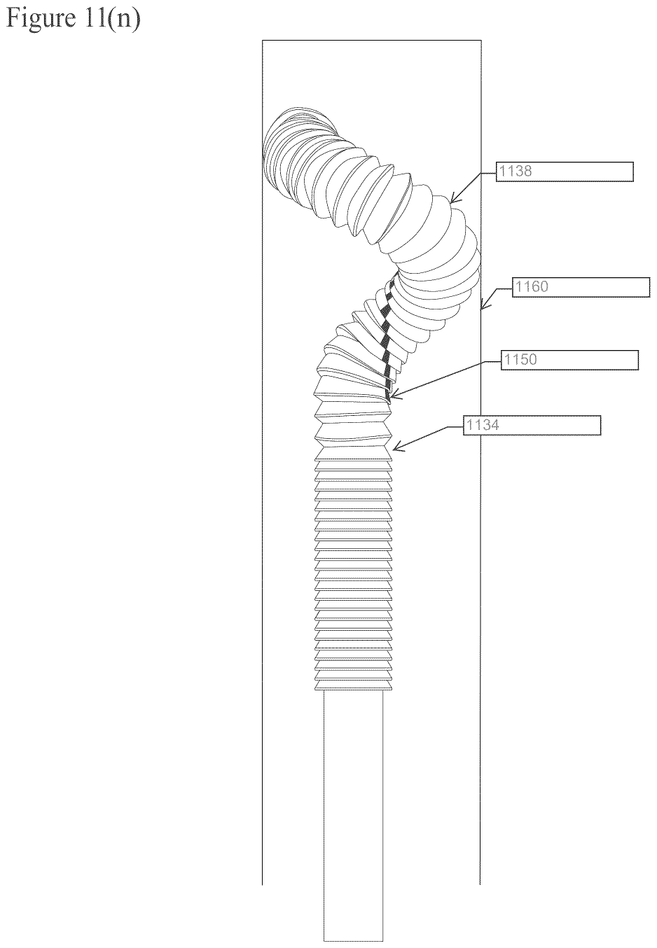

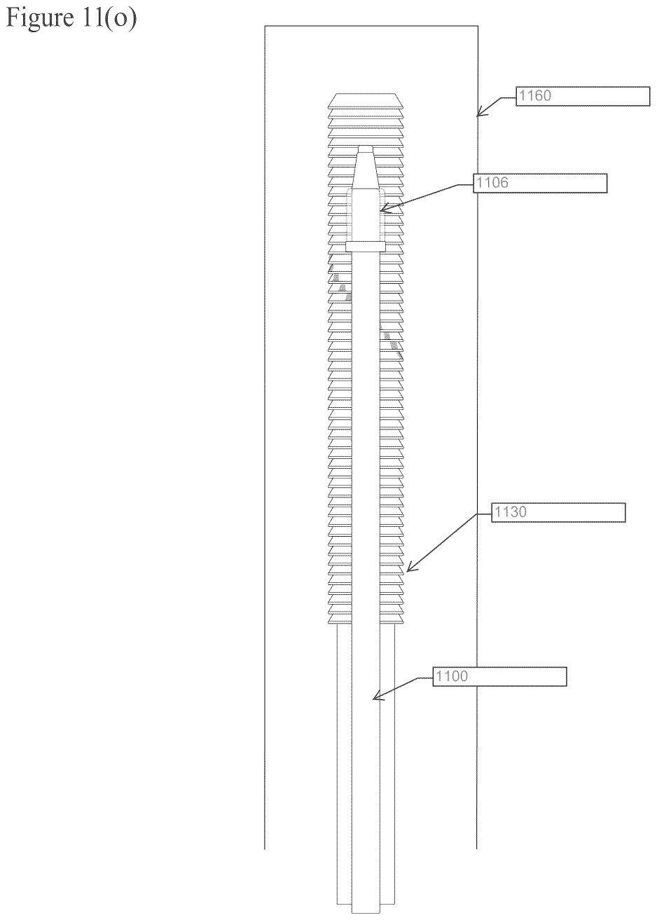

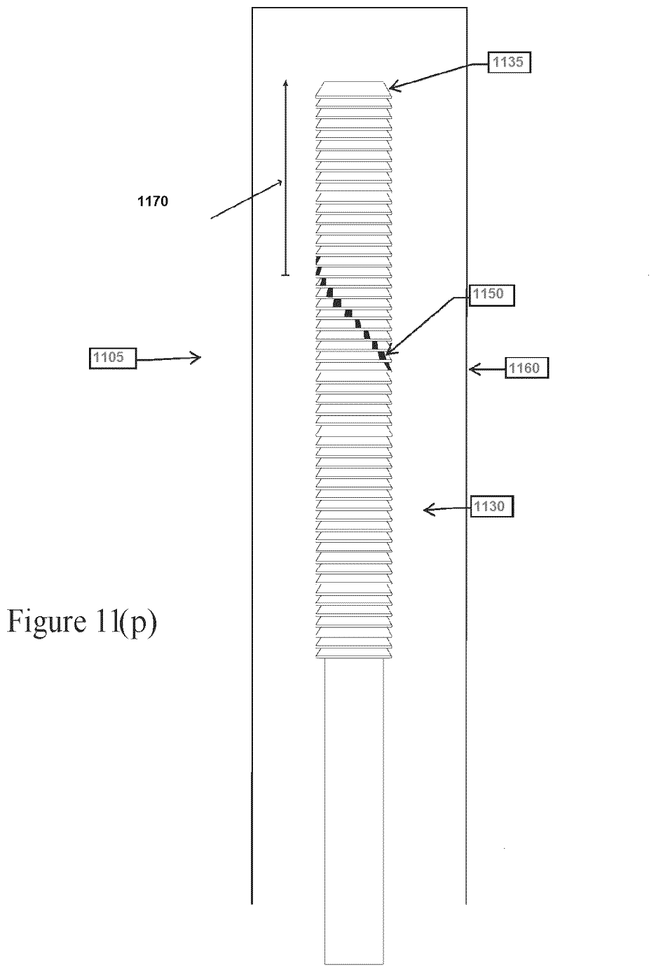

TABLE 1
Hydraulic micro-catheter shaping device (shaper) embodiments

| | Hydraulic | Maximal | | |
|---|---|---|---|---|
| | Mechanism | Extension | | |
| | # of lumens | ratio | Number of | |
| | refer to | (typical | hydraulic | Exemplary |
| # | inflation lumens | values) | Inputs | Function |
| 1 | Single inflation | 1:3-5 | 1 | Multi-arc frusta, |
| | lumen proximal | | | the frusta can be |
| | to distal (P2D) | | | predesigned for a |
| | high axial | | | set 3D curve |
| | extension | | | when actuated |
| 2 | Single inflation | 1:1.5-2 | 1 | High resolution/ |
| | lumen axial | | | Single arc, shaper |
| | extension | | | pressure is |
| | | | | controlled, |
| | | | | pressure is |
| | | | | proportional to |
| | | | | amount of |
| | | | | opened frusta |
| 3 | Single inflation | 1:1 side | 1 | Curve a |
| | lumen flexural | creasing only | | continuous stable |
| | shaper | | | arc |
| 4 | Single inflation | 1:1 side | 1 | Curve a |
| | lumen flexural | creasing only | | continuous stable |
| | shaper with | | | arc, restore |
| | spring retraction | | | original CFT state |
| | | | | when pressure is |
| | | | | released |
| 5 | Hydraulic crawling | | | See FIG. 11 |
| | type shaper. | | | |

In some embodiments of the invention, the hydraulic shaping devices described in table 1 are to be positioned along the CFT as part of a retractable micro-catheter device. This method may be especially useful in limited diameter configurations where a shaper micro-catheter can be exchanged for other transcatheter devices once the CFT catheter/sheath is desirably configured. The method is also congruent with some examples of peripheral radiological workflow, where micro-catheters (3-4 Fr) are typically used over-the-wire and in conjunction with 5-6 Fr catheters. It is noted, however, that the same methods and designs can be used also in catheter designs where the hydraulic device is non-retractable, and fixed to (or in) the CFT wall. Such permanent hydraulic devices can be designed in larger dimeter settings, e.g. cardiological/transseptal placement sheaths, as part of a CFT sheath. Optionally, such compartments are embedded in the wall of the device and no separate balloon is provided.

In addition to a desired choice of material and geometric properties for the CFT e.g., as described in the section titled “Conical Frusta tip” there are provided some alternative designs which may provide various functions. The following table summarizes some exemplary CFT embodiments (which are optionally coated and/or covered with various layers, such as for smoothing, hydrophilic behavior, biocompatibility, water proofness and/or protection):

TABLE 2

| | | Maximal | |
|---|---|---|---|
| | | Extension | |
| | | ratio (N is | |
| | | typically | |
| # | CFT type | between 1-5) | Exemplary Function |
| 1 | Unconstrained, | 1:N | The CFT can be statically |
| | PTFE coated | | shaped into any desired 3D |
| | internally | | curve and maintained stable |
| | | | in that position |
| 2 | Unconstrained | 1:N | Same as #1 but with pre- |
| | Pre-shaped, | | shaping when unactuated. |
| | PTFE coated | | The CFT can function as a |
| | internally | | statically pre-shaped sheath. |
| 3 | Constrained (e.g., | 1:N | Constraints are inherent to |
| | tied at certain | | the CFT manufacturing |
| | locations so it | | process and are optionally |
| | is restricted | | applied in designated intervals |
| | in what it can | | of θ, the azimuthal angle of |
| | do. For example, | | the frustum plane, and \hat{Z}, |
| | this allows a | | axial coordinate - spanning a |
| | predefined curve | | number of neighboring frustas |
| | to be achieved | | along the axis. When actuated |
| | using a simple | | with the first shaper in this |
| | balloon), PTFE | | table the CFT performs a set |
| | coated internally | | 3D arc which is predesigned. |
| 4 | Unconstrained, | 1:N | The elastic lining provides a |
| | PTFE coated | | shape restoring property. |
| | internally, | | When the CFT is actuated |
| | elastically | | locally it assumes the multi- |
| | lined externally | | stable arc, when pressure is |
| | | | released the CFT restores its |
| | | | original unactuated shape, |
| | | | whether pre-shaped or |
| | | | straight. The shape restoring |
| | | | mechanism can be applied |
| | | | intermittently along the axis, |
| | | | such that both multi-stable |
| | | | arcs and pressure dependent |
| | | | arcs are formed along the |
| | | | axis. |
| 5 | Unconstrained, | 1:N | Same as 4 but shape restoring |
| | elastically lined | | mechanism is internal. |
| | internally, the | | |
| | liner may | | |
| | include a PTFE | | |
| | coating as well. | | |
| 6 | Constrained, | 1:N | Same as 3 but with shape |
| | elastically | | restoring capabilities. |
| | externally/internally | | |

CFT—conical frusta tip embodiments which appear at the distal end of the catheter/sheath

In some embodiments of the invention, the sheath and/or catheter are manufactured to have a non-straight resting or starting position, for example, the CFT is bent plastically along a set curve during manufacturing so that locally slight deviations of the frusta cells generate a static curve, as in the form of a pre-shaped guide catheter (e.g., a Judkins left). This can also be understood as a slightly distorted cone section in each cell, which integrally generates a preshaped curve.

After presenting tables 1 and 2 it is emphasized that based on the section titled “Solving for the final shape of the catheter under hydraulic actuation”, one can generate many CFT designs, e.g., based on the designs and calculations methods described herein, such as using a simulation driven parametric optimization process to choose the exact hydraulic compartment diameter, the diameter and/or local cell geometry of the CFT and/or other design parameters.

In one example, one guesses an initial configuration for a specific task, e.g. set a desired 3D curve, defines simulation parameters, and after each simulation run the parameters are updated until the simulation converges to a desired output (e.g., the desired curve). This allows one to construct a shaper micro-catheter, with dedicated diameter, elastic membrane geometry and material that generates a desired curve in the CFT upon actuation, where the tip angle can also depend on the magnitude of the pressure being input to the shaper by surrounding tissues.

### Exemplary CFT Dimensions

In some embodiments of the invention, the CFT minimum wall thickness is twice the thickness of the tube material which is used to manufacture it. Generally, the minimal achievable wall thickness of an extruded tubing depends on its diameter. For example, in a 5 mm diameter tube which has a 0.1 mm wall, the CFT thickness can be 0.2 mm (extreme limit). This suggests that a portion of the lumen cross-section diameter taken up by CFT walls, can be, for example, 4%. As noted a smaller thickness can be provided, e.g., if the wall thickness is smaller. In some embodiments of the invention, the percentage of diameter taken up by the CFT, which also indicates the inner lumen diameter as a function of outer lumen is, for example, between 4% and 50%, for example, between 10% and 30%, for example, between 15 and 26%. In a particular example, the inner lumen of the CFT is 75% of the total outer diameter.

In some of the drawings herein, the CFT cells have an axial length of 0.16 units when collapsed and 0.36 units when open. This gives an extension ratio of 2.25 where the distance is measured as the distance between the extreme bounds of the upper and lower conical frusta surfaces comprising each cell—710 and 712 in FIG. 7(b). It is noted that higher extension ratios can be achieved, for example, 1:3, 1:5, 1:10 or smaller or intermediate ratios, as well as smaller ratios, such as 1:1.5-1:2 and/or 1:2-1:3. In some embodiments of the invention, a collapsed cell height (radially measured) is for example, between 0.1 mm and 10 mm.

### DESCRIPTION OF EXEMPLARY DRAWINGS

### FIG. (Set) 1—CFT Sheath/Catheter and OTW (Double Lumen) Flexural Shaper Micro-Catheter—Shaping a Curve in the CFT

This figure set shows an exemplary process of shaping a two dimensional curve in the CFT by a double lumen micro-catheter shaping device (shaper of type 5—see table 1 and CFT of type 1—see table 2), in accordance with some embodiments of the invention.

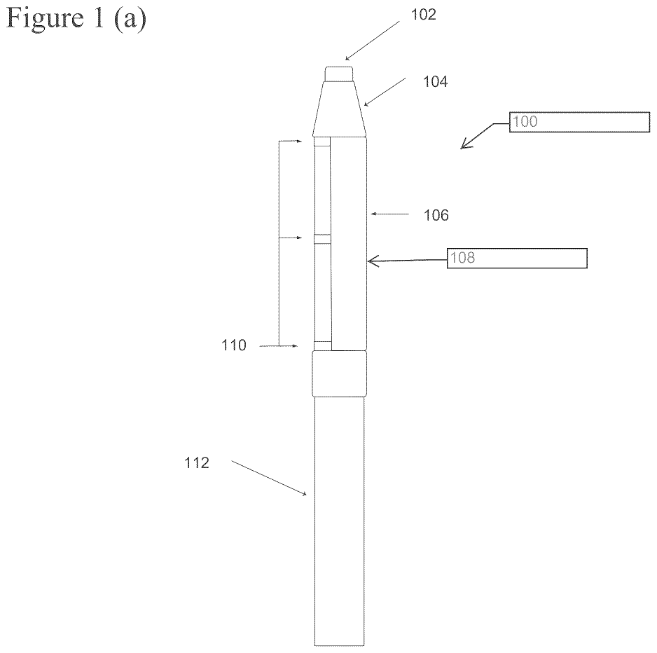

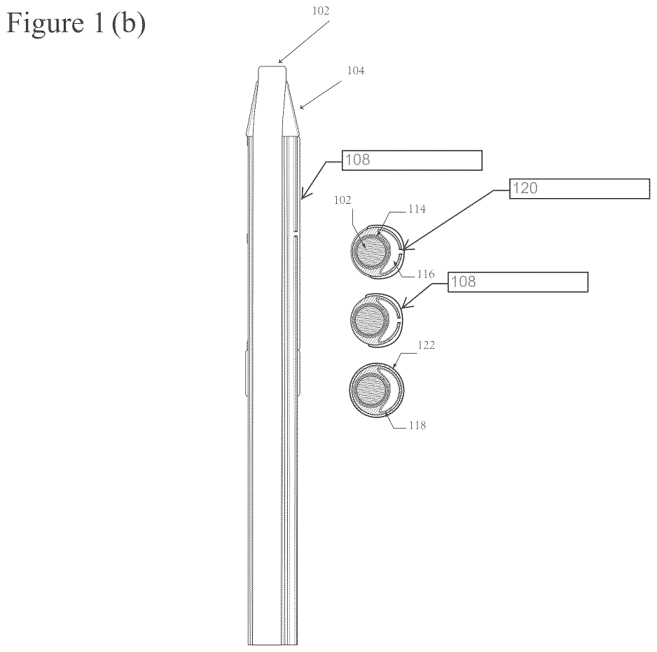

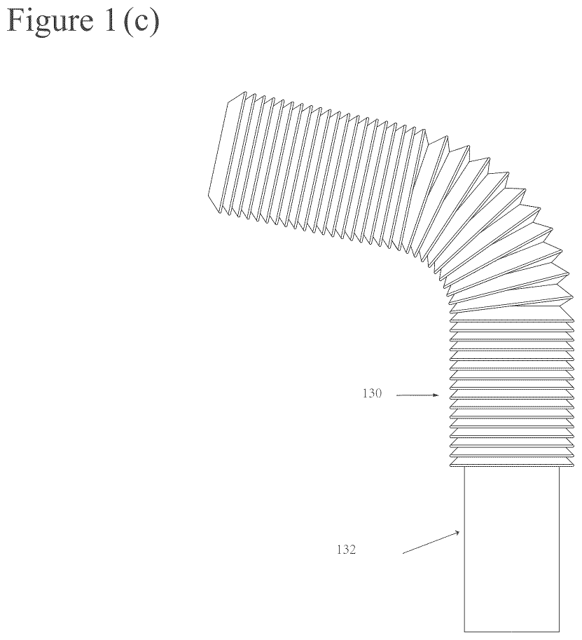

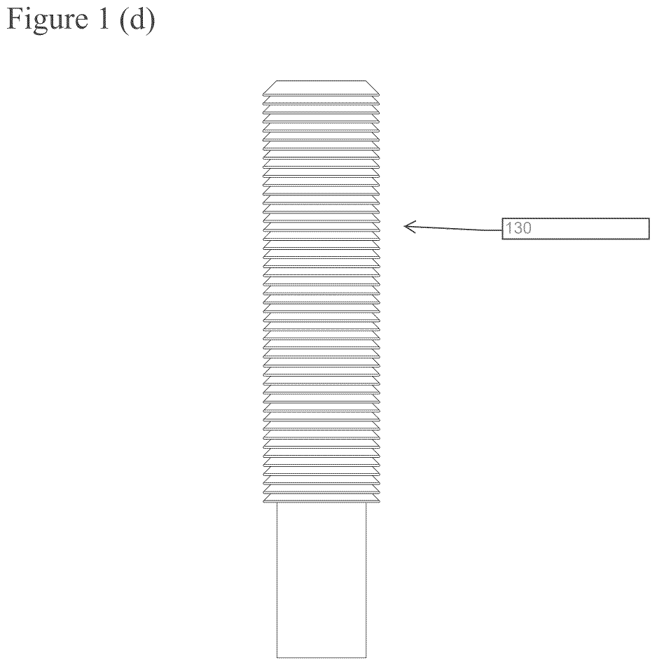

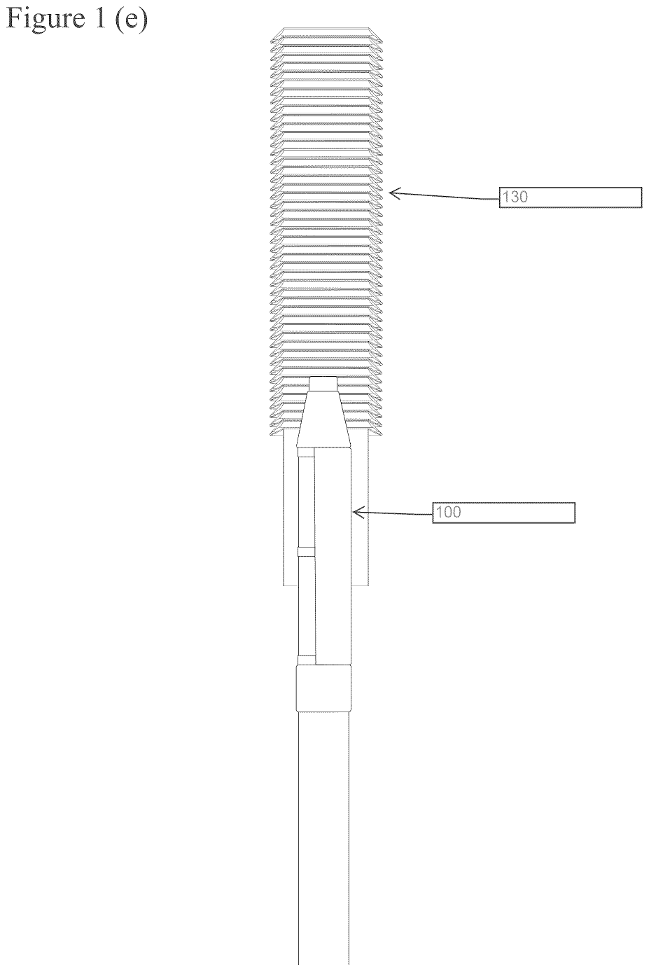

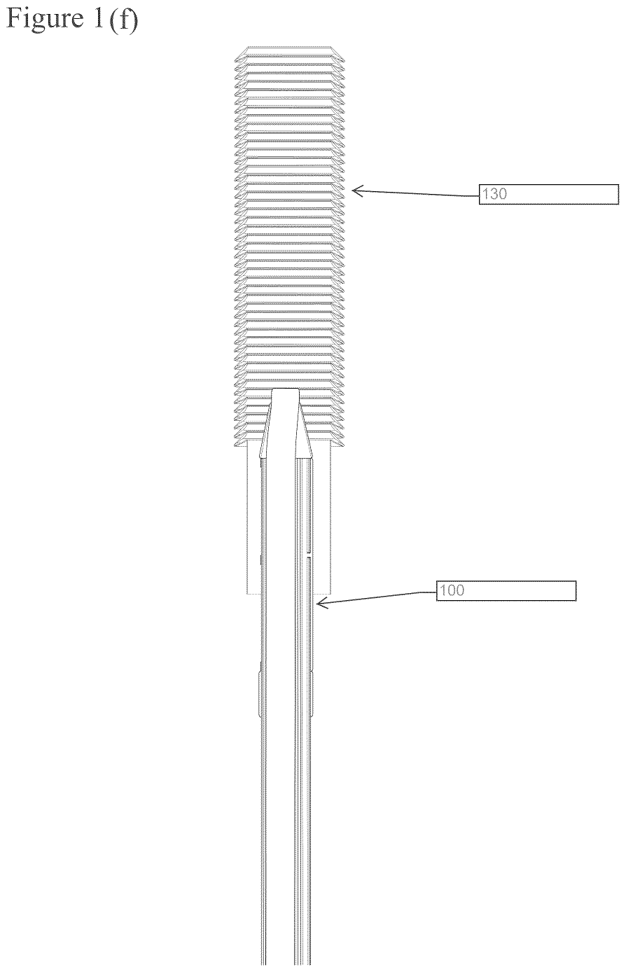

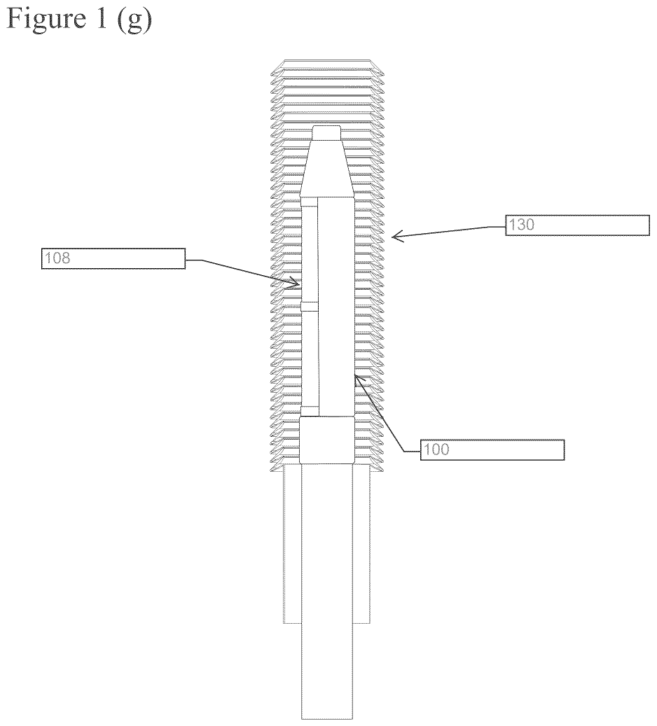

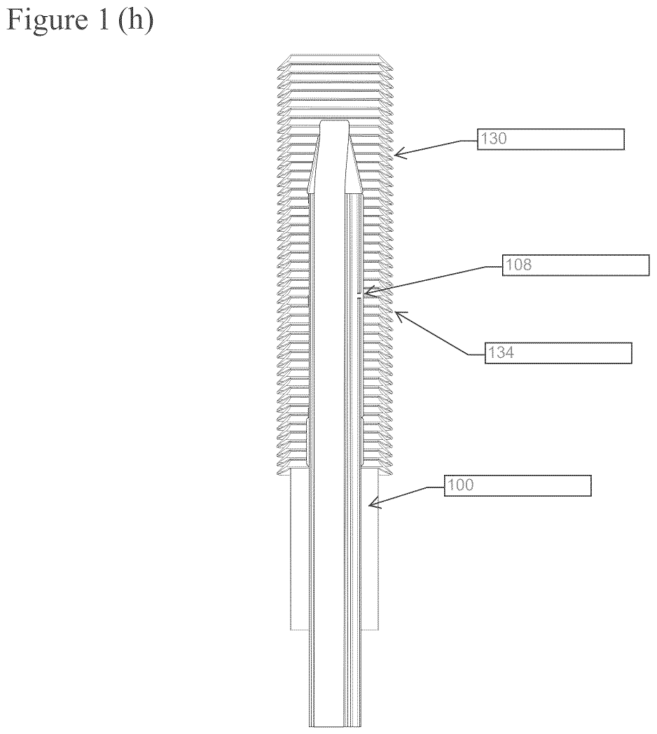

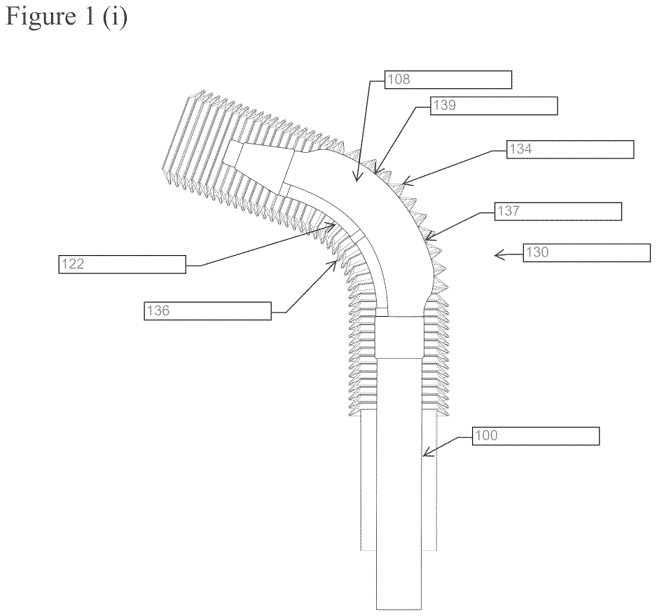

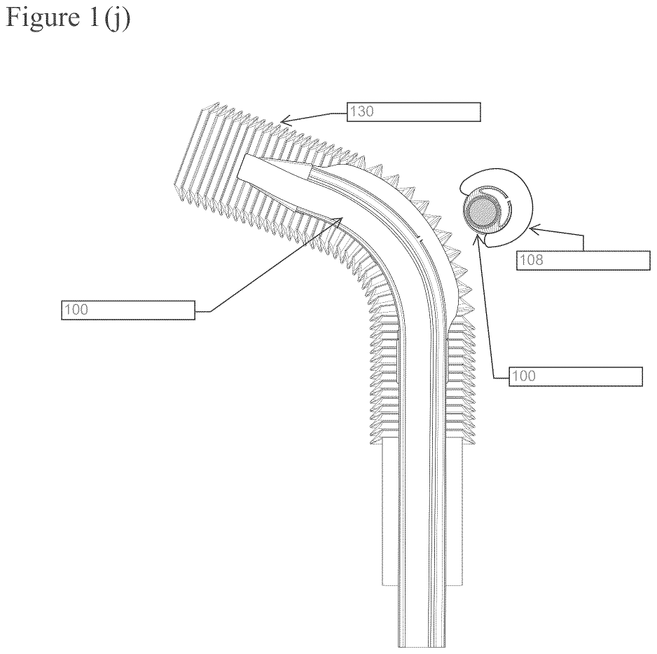

FIG. 1(a)—an exemplary two-dimensional side-view of a double lumen (OTW) shaper 100, optionally a microcatheter. A wire 102 is seen protruding a tip 104 of the shaper.

Exemplary Hydraulic Device Construction (See FIG. 1(a))

In some embodiments of the invention, a hydraulic device core compartment 106 includes an inflatable elastic cylindrical tube or membrane 108 and is designed to form a curve in the CFT. An exemplary method of operation is by latching membrane 108 (e.g., using a friction mechanism and/or a geometrical interference mechanism, based for example on protrusions) radially and asymmetrically onto the CFTs internal wall while distancing (or bringing together) any two material points along the longitudinal axis away from each other. Once pressurizing the membrane, a strain field begins to form almost exclusively in the surface being designed to yield first (optionally membrane 108 is fabricated with a changing yielding or thickness function (e.g., see FIG. 7 (d)—the thinner wall areas will yield first and areas with a thicker walls will yield later). Referring specifically to FIG. 1(i), when membrane 108 expands radially, it contacts the inner surface of a CFT bending section 134 at a plurality of points, for example, points 137 and 139. This contact provides friction between membrane 108 and points 137, 139. In some embodiments, membrane 108 includes one or more protrusions or is forced by expansion to match (e.g., mechanically interfere with relative axial motion therebetween) an inner geometry of section 134. Further expansion is constrained radially by the CFT itself and membrane 108 now expands axially. Points 137 and 139 are carried along due to the friction and a distance between them increases (as membrane 108 is expanding axially also between points 137, 139). This applies sufficient force to CFT cells at section 134 (and between points 137, 139) to cause a state change. As this effect is asymmetric, it causes bending of CFT 130 as a whole, at section 134.

Optionally, the face of membrane 108 designed to yield first is hereafter termed the “convex face”, the opposite side is hereafter termed the “concave face”. Membrane 108 inflates and touches the CFTs internal wall, and the mechanism of curving the CFT takes effect. The design optionally includes one or more radio-opaque markers 110 on the concave face of membrane 108 and which can provide imaging (e.g., x-ray) feedback as to the location of the shaper along the CFT and/or as to the curvature of the membrane. Optionally or additionally, the CFT includes such markers. A shaft 112 of shaper 100 is optionally coated with a hydrophilic layer.

FIG. 1(b)—shows axial and cross-sectional view of shaper micro-catheter 100 with two visible lumens 114 (guidewire lumen), 116 (inflation lumen) in accordance with some embodiments of the invention. Inflation lumen 116, optionally defined by a hypotube 118, optionally includes one or more orifices 120 to supply pressurized saline to membrane 108 which is optionally elastic. Element 122 indicates a lock-seal section of the catheter, which optionally serves to seal the membrane to the body of the catheter and limit the axial extent of expansion of membrane 108. An axial cross-sectional view of the shaper construction is also viewed to the right of the radial cross-sections describing a top cross-sectional view of the above.

FIG. 1(c)—two-dimensional side view of a CFT 130 after actuation, in accordance with some embodiments of the invention. This is an exemplary desired stable shape that the CFT will be formed into, in the following steps (FIGS. 1(d)-1(j)) which will describe the process. Section 132 indicates an elongate non-CFT body portion which may be braided or transition to braiding or other body structure. A diameter of section 132 optionally corresponds generally to the inner diameter of CFT 130. During the manufacturing process CFT 130 is optionally blended (e.g., welded or worked from a same tube) with a desired shaft section 132.

FIG. 1 (d)—two-dimensional side view (radial) of CFT 130 in an unactuated state, in accordance with some embodiments of the invention. The CFT shown is a particular case of the CFT geometry. Its extension ratio is 1:2.25.

FIG. 1(e)—radial cross-section view of CFT 130 along with a two-dimensional side-view (radial) of shaper micro-catheter 100 positioned at a proximal end of CFT 130.

FIG. 1(f)—radial cross-section view of CFT 130 along with a radial cross-section view of shaper micro-catheter 100 positioned at a proximal end and within CFT 130.

FIG. 1(g)—radial cross-section view of CFT 130 along with a two-dimensional side-view (radial) of shaper micro-catheter 100 as it is advanced axially into CFT 130 and positioned (e.g., membrane 108 thereof) at a desired location where shaping of CFT 130 is desired, in accordance with some embodiments of the invention.

FIG. 1(h)—radial cross-section view of CFT 130 along with a radial cross-section of shaper micro-catheter 100 positioned at a desired location and rotated so that membrane 108 faces a section 134 of CFT 130 to be selectively axially extended, thereby causing a local bend, in accordance with some embodiments of the invention. In some embodiments of the invention, the bending section 134 is between 3 and 70 mm long, for example, between 10 and 40 mm long. This may be set by the length of membrane 108 and/or actual length of CFT containing parts. So, for example, membrane 108 (or other membranes described herein) may have a length (e.g., when expanded) of between 10 and 300 mm, for example, between 20 and 100 mm.

FIG. 1(i)—radial cross-section view of CFT 130 along with a two-dimensional side-view (radial) of shaper micro-catheter 100 as it is actuated hydraulically so membrane 108 expands, in accordance with some embodiments of the invention. Upon actuation CFT 130 is optionally curved about the center of membrane 130, so as to cause a convex curve in a section 134 of CFT 130 and a concave curve in a section 136 of CRT 130. Optionally, an inner curve section 122 of shaper 100 is pressed against section 136, to provide a counter-force to the contact of membrane 108 against an inner wall of section 134. This may help ensure friction between membrane 108 and section 134 and/or ensure delivery of radial force to section 134.

FIG. 1(j)—radial cross-section view of CFT 130 along with a radial cross-section of shaper micro-catheter 100 as it is actuated hydraulically, in accordance with some embodiments of the invention. An axial cross section is also shown in the figure (right) in which shaper's membrane 108 can be seen when actuated. Optional tapered (soft) portion 104 of the distal end of optionally can easily be curved along with CFT 130. Upon actuation, CFT 130 is optionally curved about the center of membrane 108 (center of curvature generally coincides with axial center of a uniform membrane).

### FIG. (Set) 2—CFT Sheath/Catheter and OTW (Double Lumen) Extension Shaper Micro-Catheter

The figure set shows the process of shaping two distinct local extensions in a CFT 230, in accordance with some embodiments of the invention. The mechanism shown in the figure can be used to form a precise extension with single frustum resolution of CFT 230 controlled by saline pressure of a shaping device 200. The shaper and CFT used here are optionally shaper of type 3—see table 1 and CFT of type 1—see table 2, respectively. It is noted that such extension mechanism need not be used only for a steerable sheath/catheter application but can also be relevant to guide catheter shaft extensions, such as the Guideliner (by Teleflex, see US20180161547A1) or Guidezilla (by Boston scientific, see US20140052097A1). Reference numbers generally reflect similar or corresponding components to those in FIG. 1 and other figure sets, except that the prefix of the number is adjusted to match the Figure number. Such elements may not be described again, to reduce redundancy.

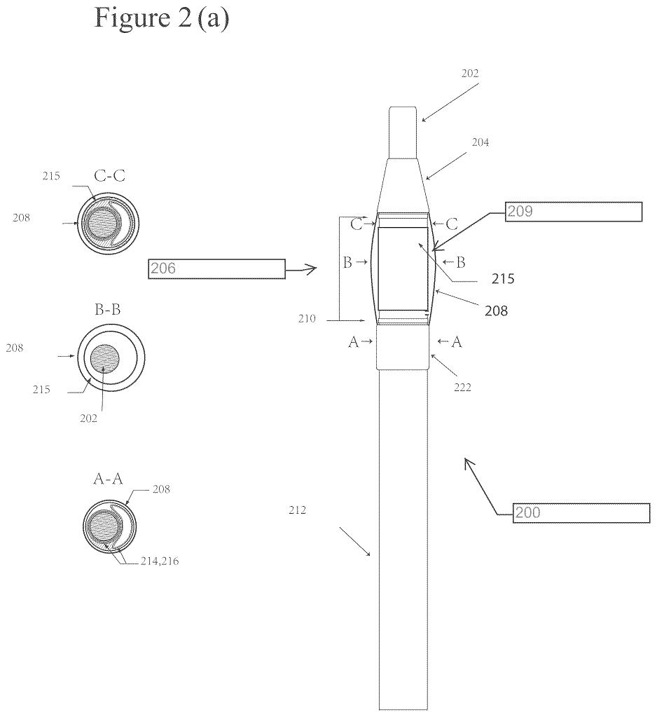

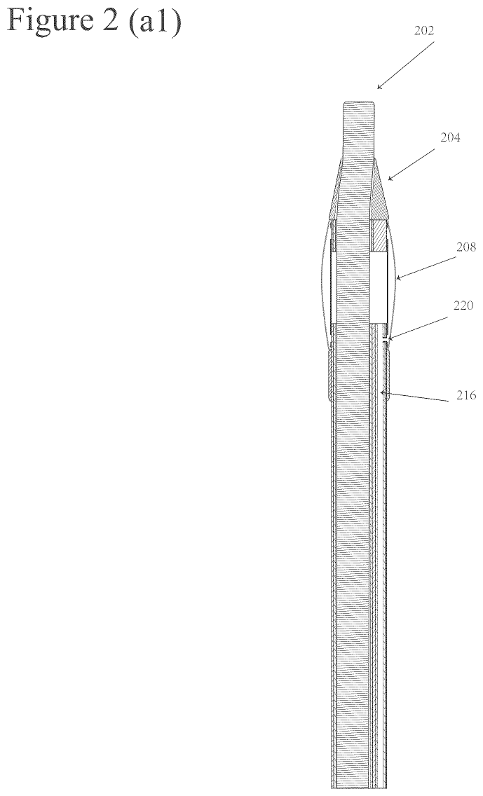

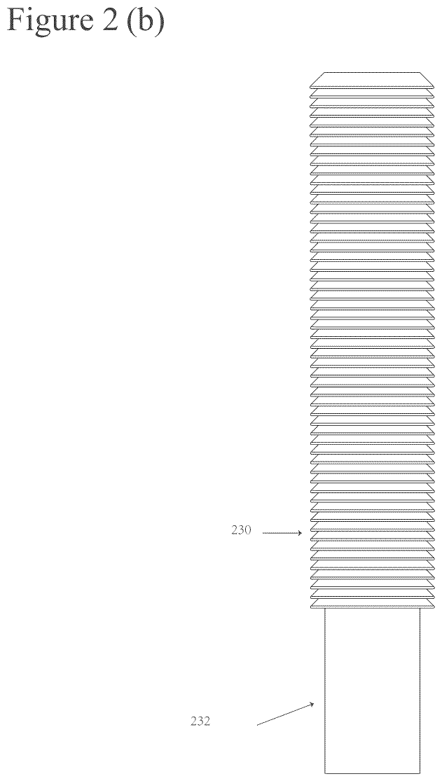

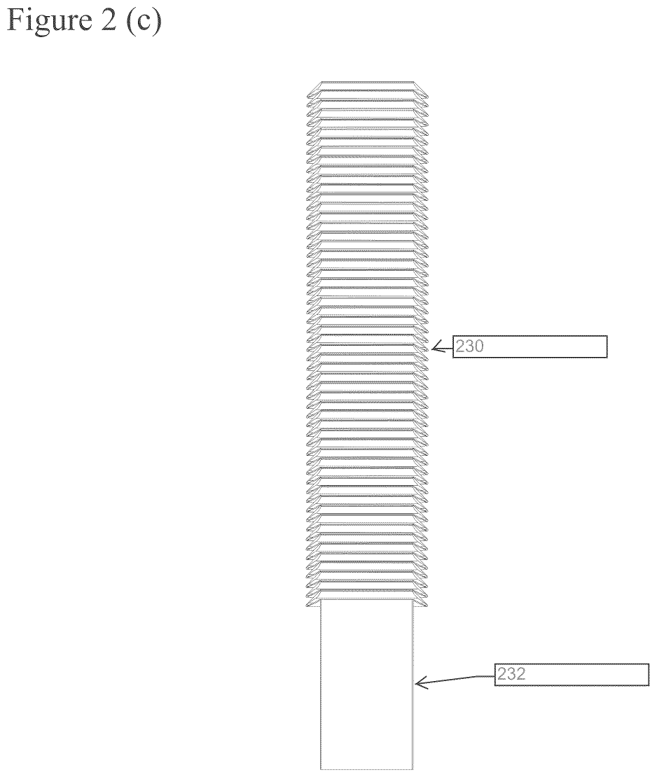

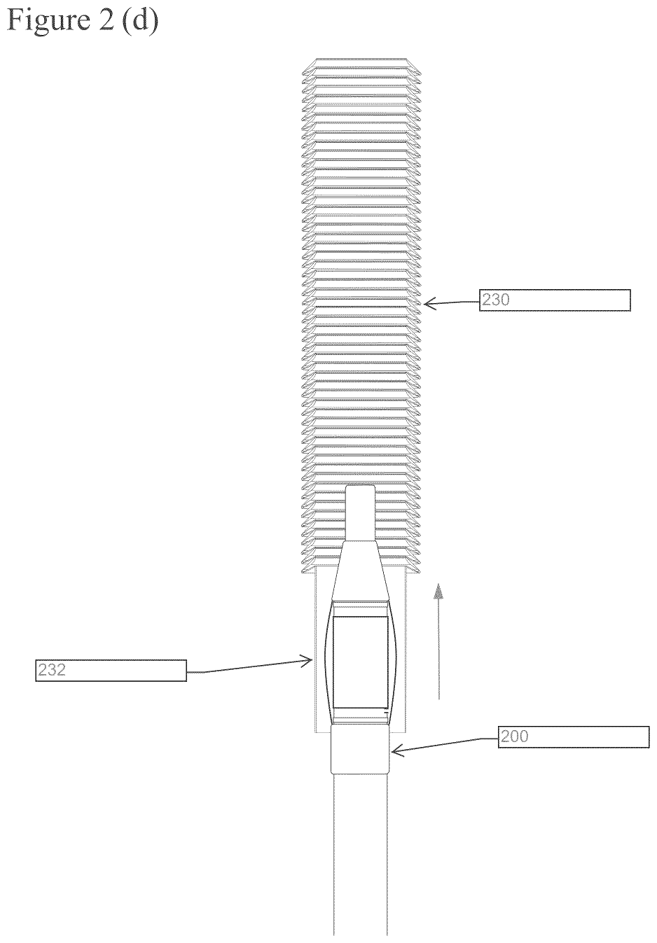

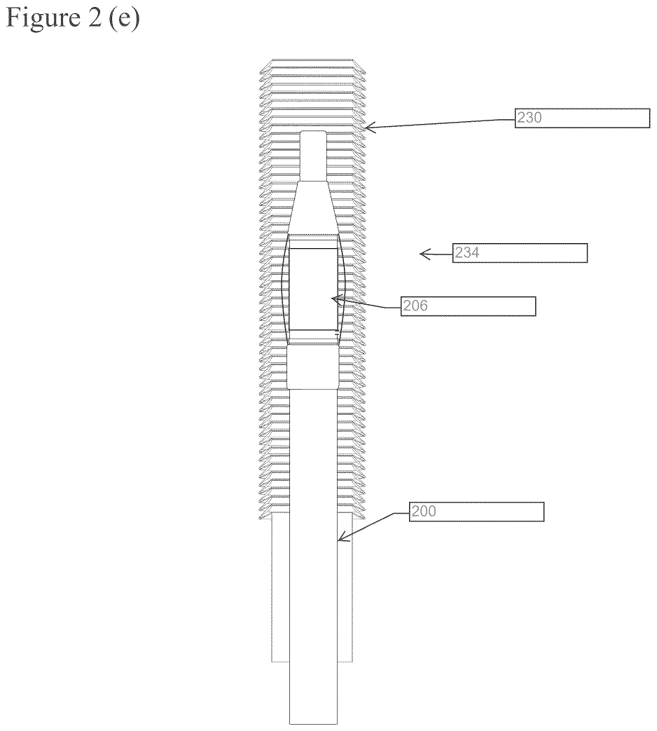

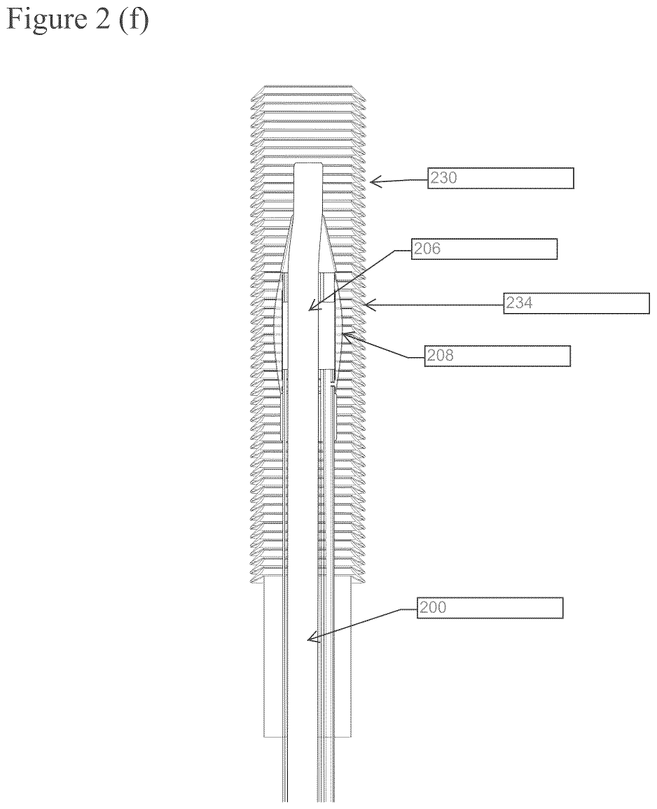

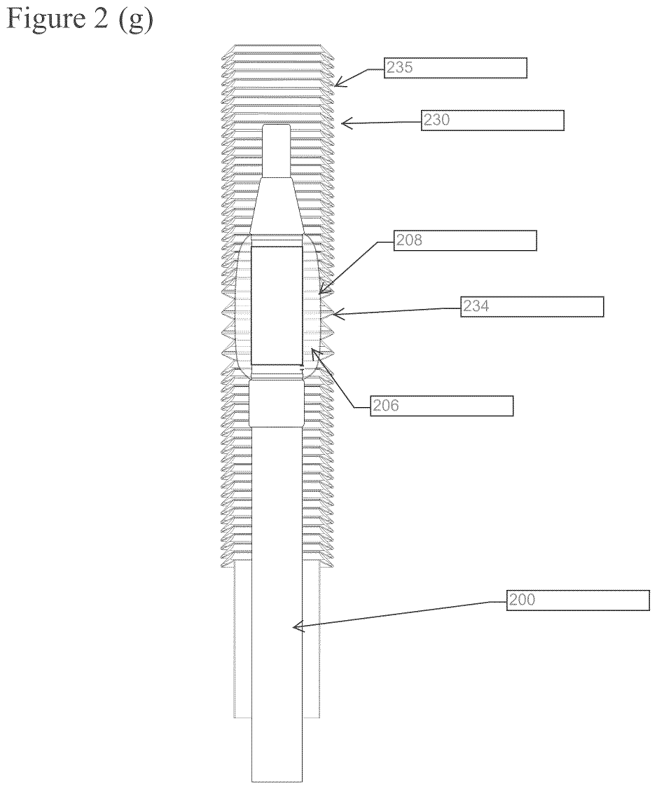

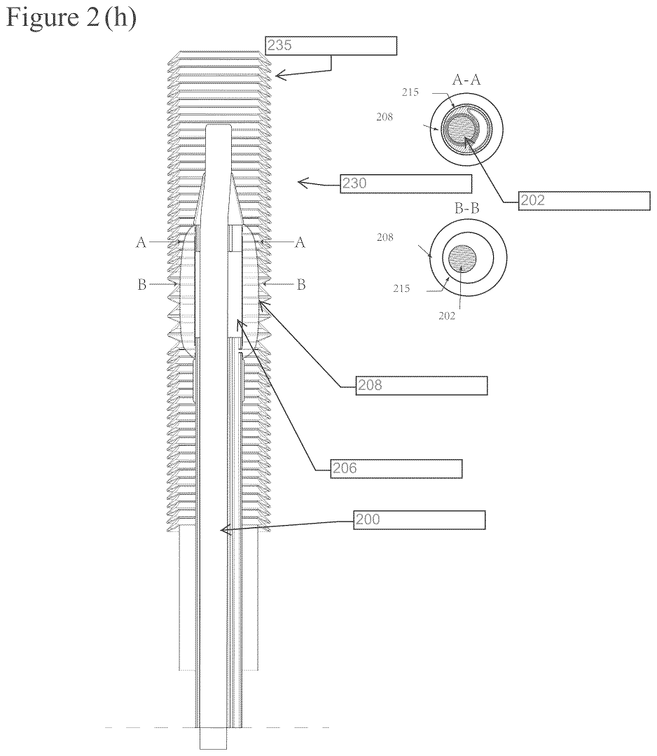

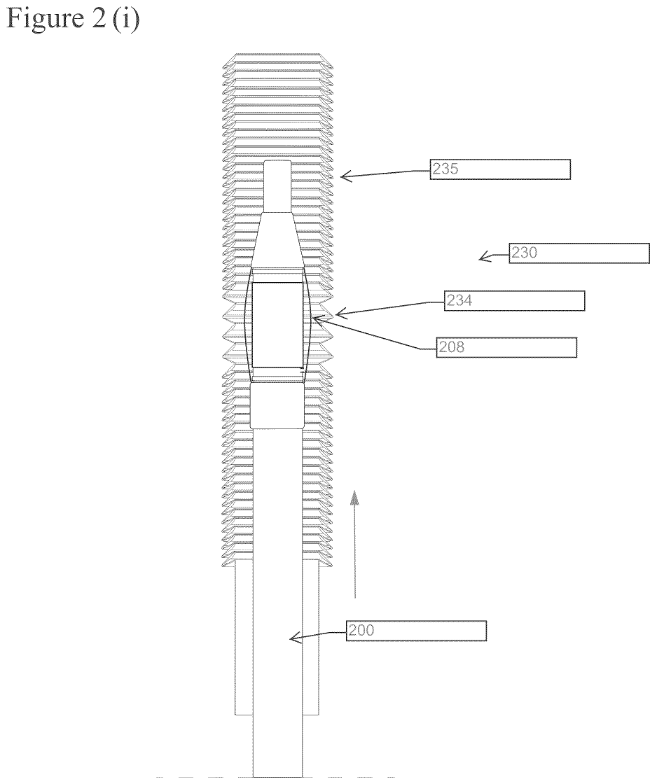

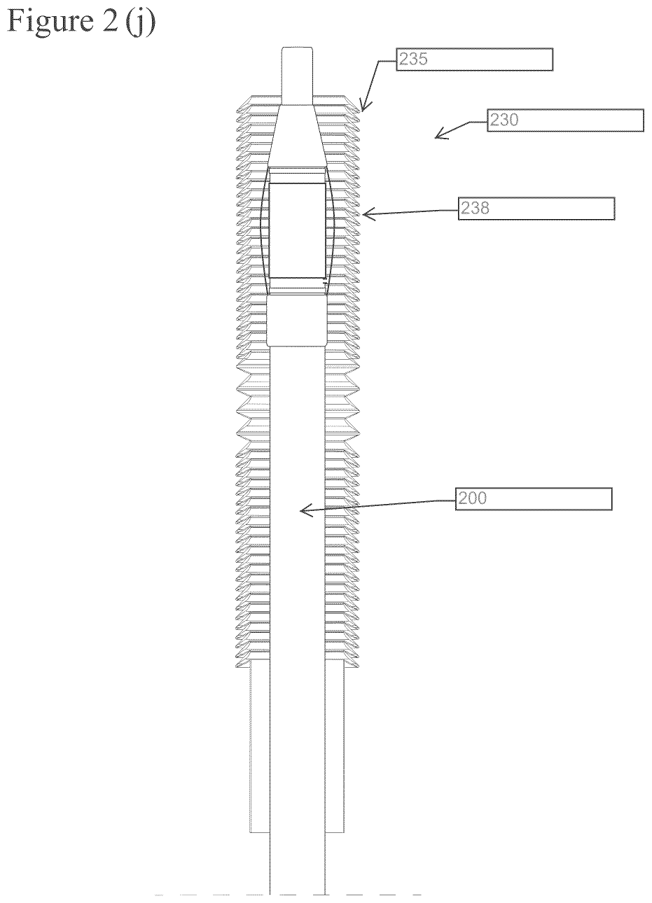

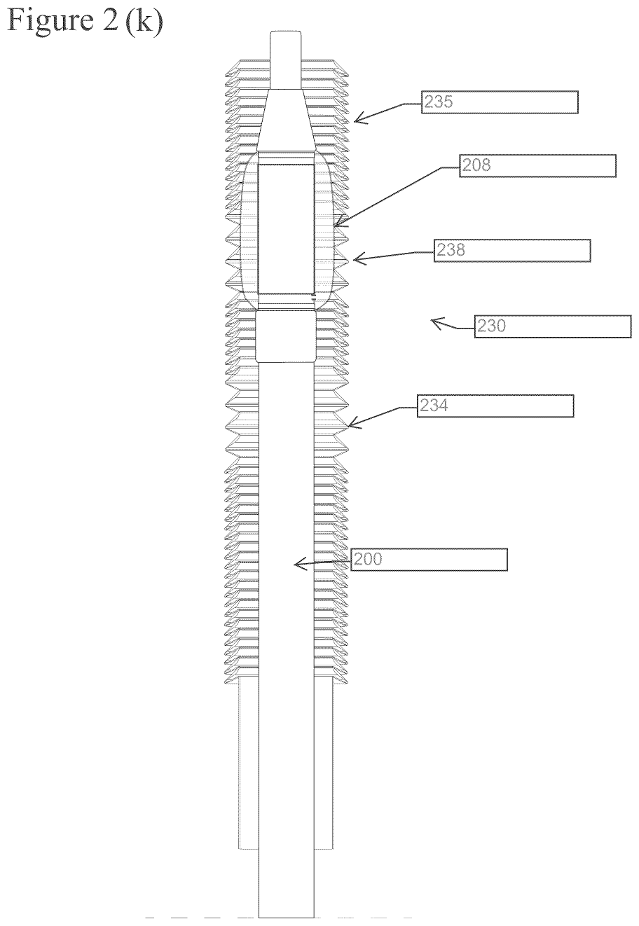

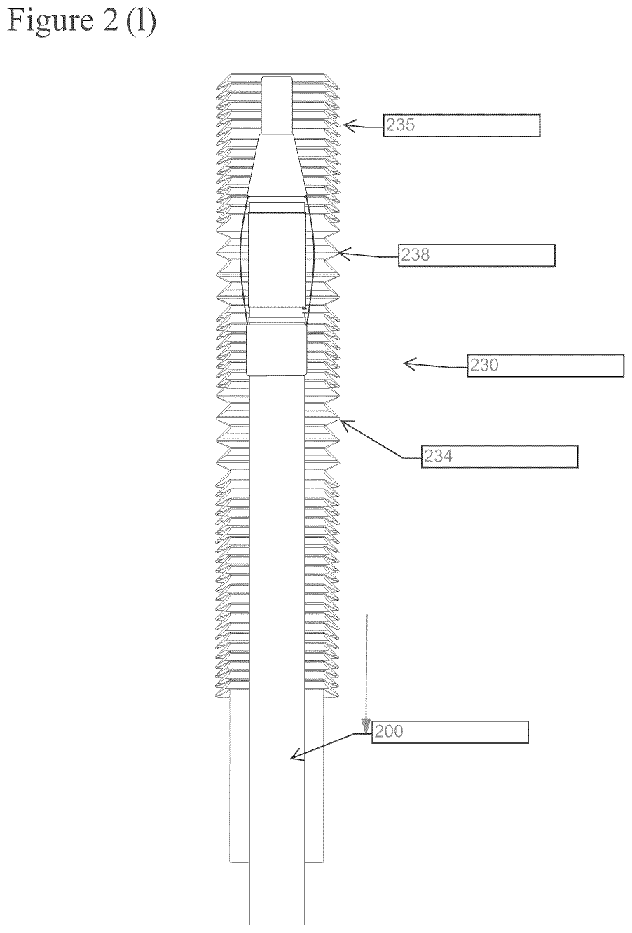

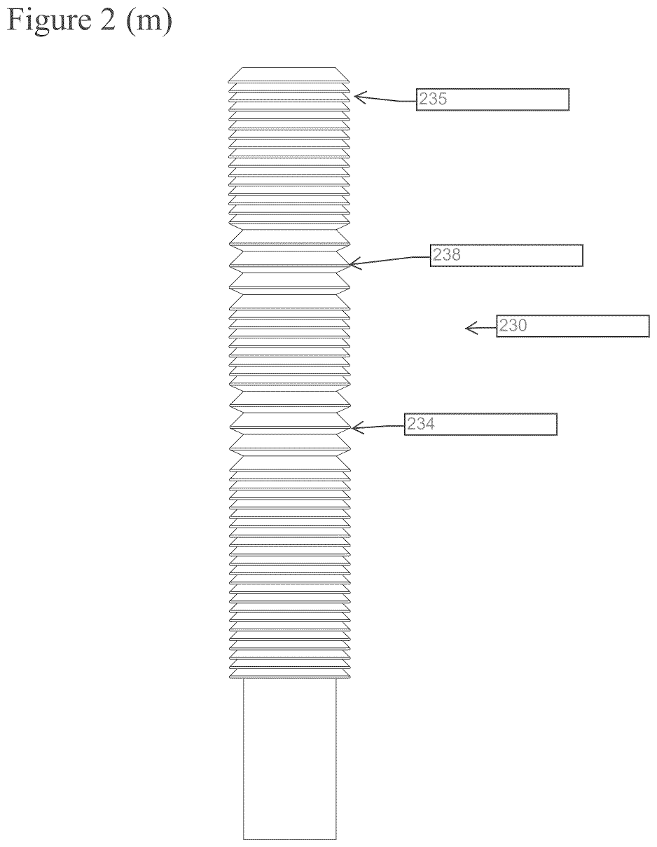

FIG. 2(a)—two-dimensional side-view of a double lumen (OTW) shaper 200and axial cross-section of a shaft 212 thereof, in accordance with some embodiments of the invention. An optional guidewire 202 is seen protruding a tip 204 of shaper 200. In some embodiments of the invention, shaper 200 at a distal end thereof includes a membrane 208, optionally elastic, designed to cause extension of CFT 230 by latching on to an internal wall thereof while distancing two material points away from each other. The strain field is optionally formed uniformly and axi-symmetrically along the circumference of membrane 208 thereby extending the CFT correspondingly. The design optionally includes one or more radio-opaque markers 210 at the extremities of the axisymmetric membrane which can provide imaging feedback as to the location of shaper 200 (and its membrane segment 208) along CFT 230 and/or as to the segment being extended. Axial cross-section views of the shaper micro-catheter are also shown in the figure including two visible lumens, in accordance with some embodiments of the invention: wire lumen 214 and inflation lumen 216. An inflation orifice between lumen 216 and a space 209 optionally supplies pressurized saline to membrane 208.

Exemplary Hydraulic Device Construction (See FIG. 2(a))

In some embodiments of the invention, the Hydraulic mechanism of shaper 200 includes an inner stretchable elastic sleeve 215, which enables the stable passage of an optional guidewire, for example when the sleeve is curved through CFT 230. Membrane 208 is optionally stretched axi-symmetrically around sleeve 215. Pressurizing membrane 208optionally causes it to undergo a combination of radial strain, which may provide at least part of the latching force onto the inner wall of the CFT, and/or optional axial strain which provides the extension force of the CFT. Optionally, shaper 100 is designed around two working pressures between which single frustum resolution can be achieved.

FIG. 2(a)—an exemplary two-dimensional side-view of double lumen (OTW) shaper 200. Wire 202 is seen protruding from tip 204 of shaper 200. The figure includes 3 axial cross-sections: A-A taken at a proximal seal of the hydraulic compartment, B-B taken amid an hydraulic compartment 206 thereof and C-C taken just proximally to a distal seal 222 of the hydraulic compartment.

FIG. 2(a1)—a radial cross-section view of shaper micro-catheter 200 with two visible lumens and also showing an inflation orifice 220, in accordance with some embodiments of the invention.

FIG. 2(b)—two-dimensional side view (radial) of CFT 230 in an unactuated state, in accordance with some embodiments of the invention. The CFT shown is a particular case of the CFT geometry. Its extension ratio is 1:2.25.

FIG. 2(c)—radial cross-section view of CFT 230—unactuated state.

FIG. 2(d)—radial cross-section view of CFT 230 along with a two-dimensional side-view (radial) of shaper micro-catheter 200 positioned at a proximal end of CFT 230.

FIG. 2(e)—radial cross-section view of CFT 230 along with a two-dimensional side-view (radial) of shaper micro-catheter 200 positioned distally so that a hydraulic actuator 206 thereof is at a first point of interest 234, where actuation is desired, in accordance with some embodiments of the invention.

FIG. 2(f)—radial cross-section view of CFT 230 along with a radial cross-section view of shaper micro-catheter 200 positioned with hydraulic actuator 206 at first point of interest 234, where actuation of membrane 208 is desired, in accordance with some embodiments of the invention.

FIG. 2(g)—radial cross-section view of CFT 230 along with a radial cross-section view of shaper micro-catheter 200 positioned distally and with hydraulic compartment 206 thereof actuated at first point of interest 234, so membrane 208 expands and applies force to an inside wall at 234. Actuation optionally generates a response in CFT 230 shown illustratively in the axial opening of 3 frusta, whereby a distal portion 235 of the CFT 230 advances correspondingly. This potential response may be provided as a result of a combination of radial and axial strain in shaping device membrane 208.

FIG. 2(h)—radial cross-section view of CFT 230 along with a two-dimensional side-view (radial) of shaper micro-catheter 200 positioned distally and actuated at first point of interest 234, in accordance with some embodiments of the invention. An axial cross-section of the shaping device is shown to the right of the figure while in the actuated state.

FIG. 2(i)—Following the actuation in FIGS. 2(g) and 2(h) shaping device 200 is now depressurized at the location of actuation (234), an arrow to the right of the figure signifies the direction in which the shaper micro-catheter may to be advanced.

FIG. 2(j)—The shaper micro-catheter is advanced to a second (distal) point of interest 238 where axial extension is desired, in accordance with some embodiments of the invention.

FIG. 2(k)—A similar process as to the one described in FIG. 2(g) takes place at second distal location 238. Distal portion 235 of CFT 230 is optionally advanced further by the same increment as in FIG. 2(g). Distal portion 235 of CFT 230 has now been extended, for example, by 6 frusta relative to its initial state prior to actuation (FIG. 2(f)).

FIG. 2(l)—Following the actuation of FIG. 2(k), shaping device 200 is now optionally depressurized at secondary location of actuation 238, a blue arrow to the right of the figure signifies the direction in which shaper micro-catheter 200 may be retracted.

FIG. 2(m)—two-dimensional side view (radial) of CFT 230 after it has been actuated hydraulically in the two designated locations 234, 238, in accordance with some embodiments of the invention.

### FIG. (Set) 3—CFT Sheath/Catheter and OTW (Double Lumen) Flexural Shaper Micro-Catheter—Segmental Shaping of a Double Curve in the CFT

Following FIG. 1, this figure set shows an exemplary process of shaping a two dimensional double curve in a CFT 330 by a two step process of successive single curves (as in FIG. 1) and alternating twist of a shaper MC (micro-catheter) 300. While the figure describes a two-dimensional curve (where the shaper is twisted 180 deg between curves), multi-plane curves can be established with the same ease and by the same procedure without any loss of generality. As in FIG. 1, a double lumen micro-catheter shaping device (shaper of type 5—see table 1 and CFT of type 1—see table 2) is optionally used.

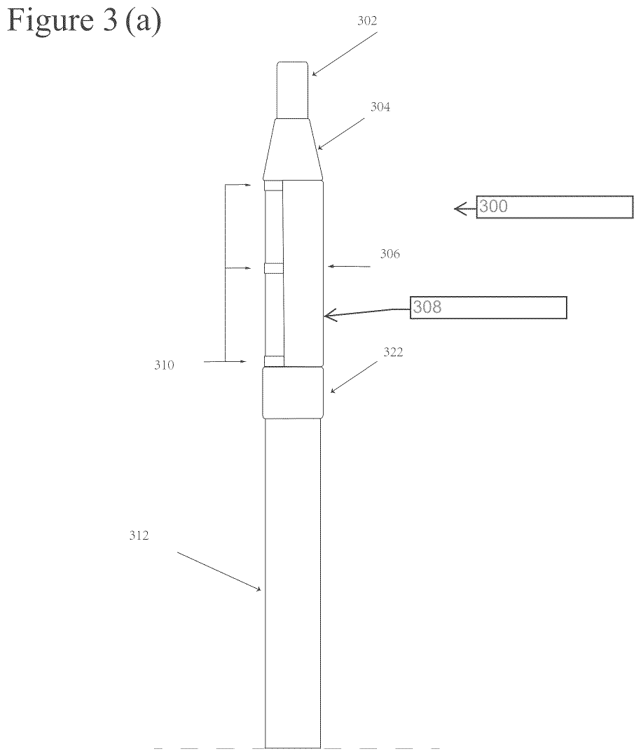

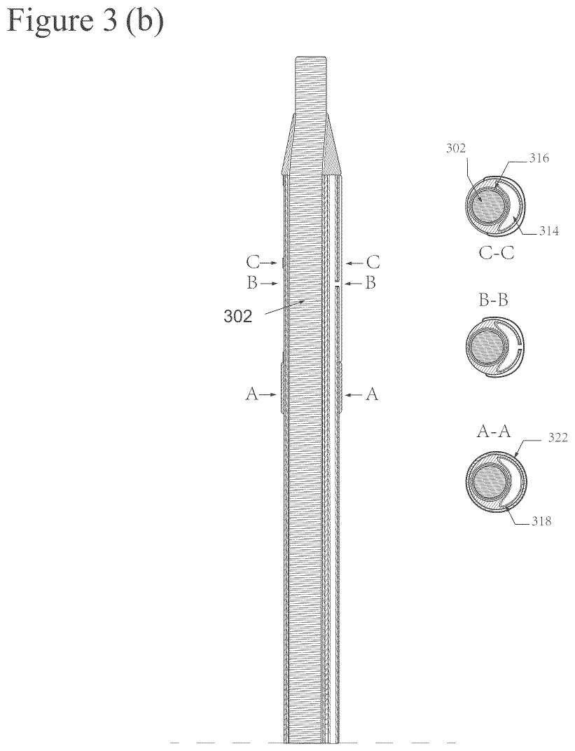

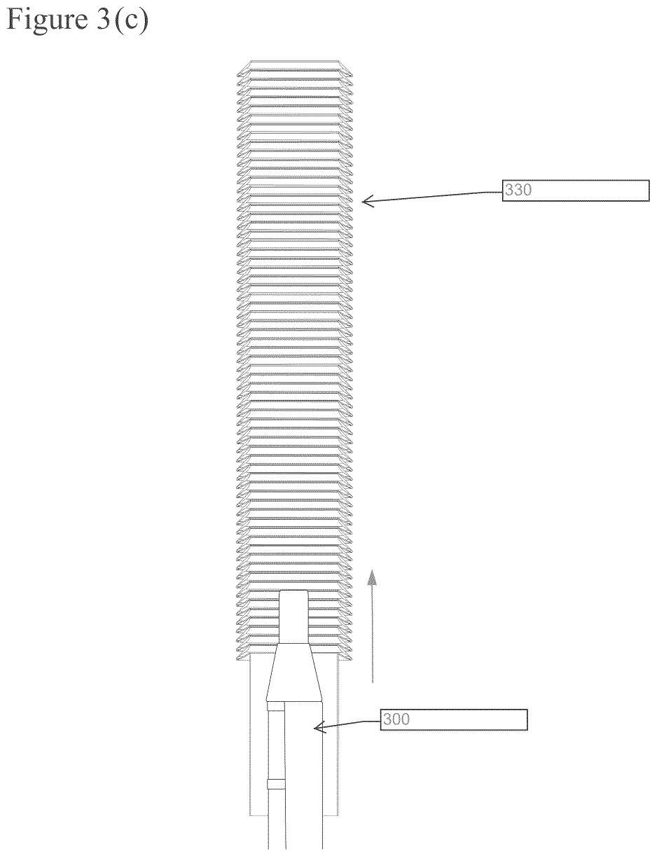

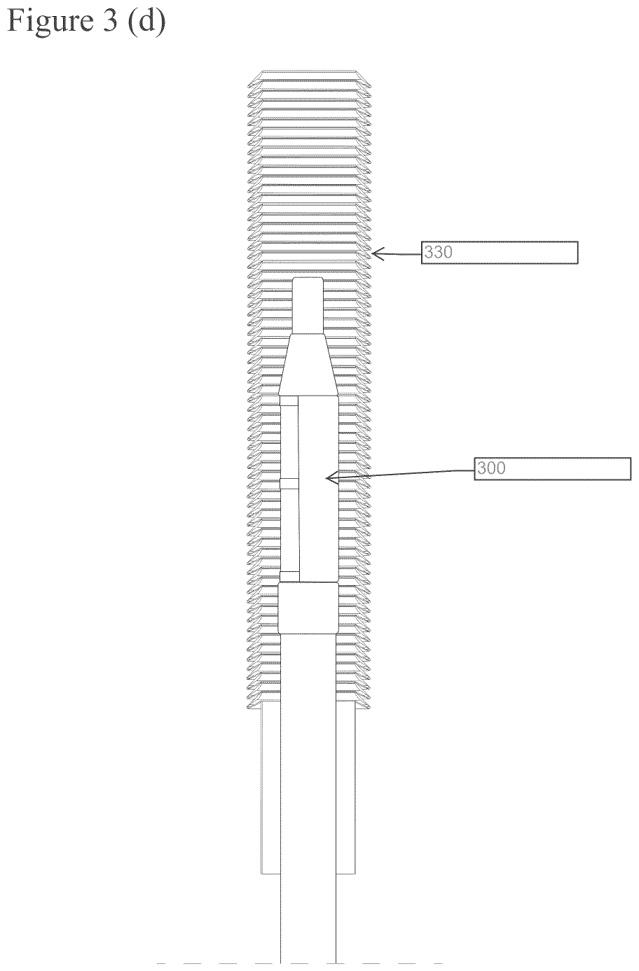

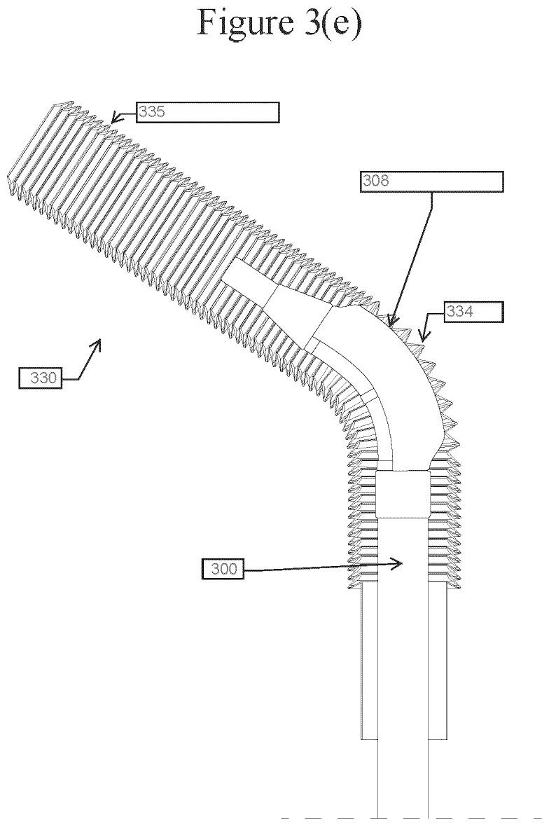

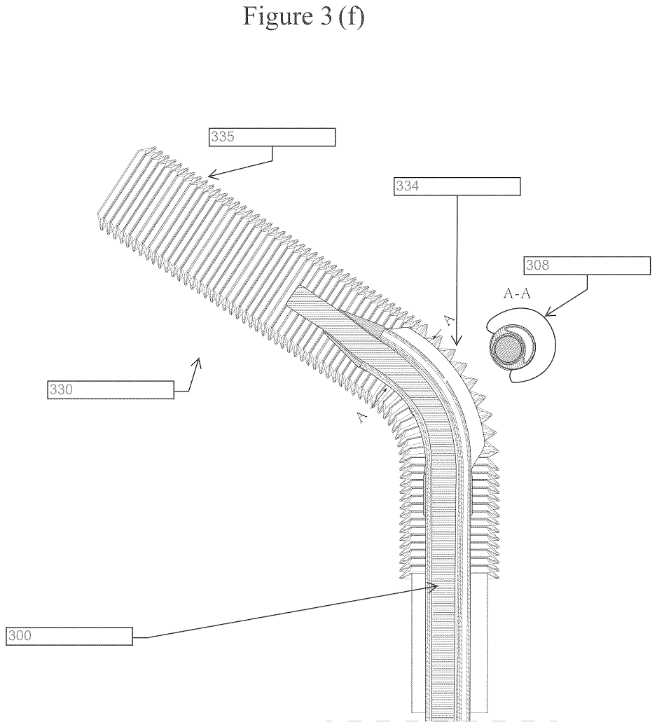

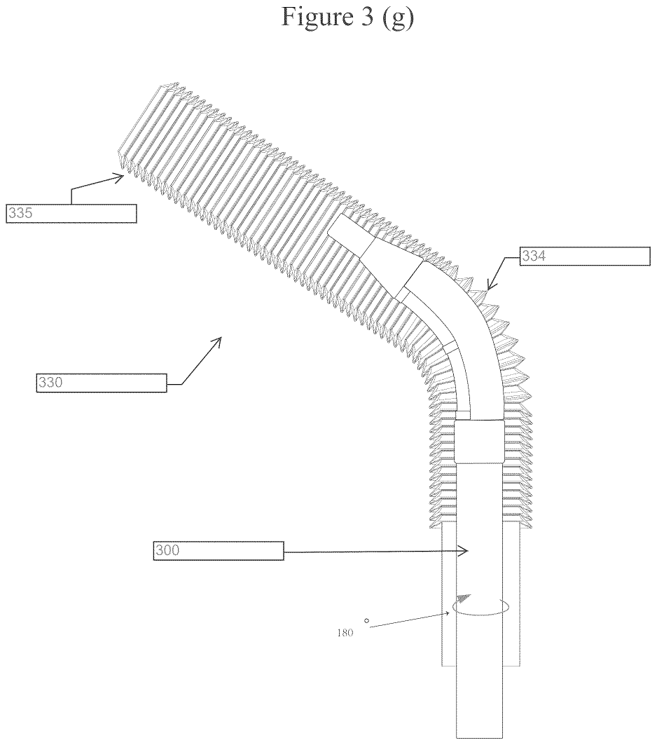

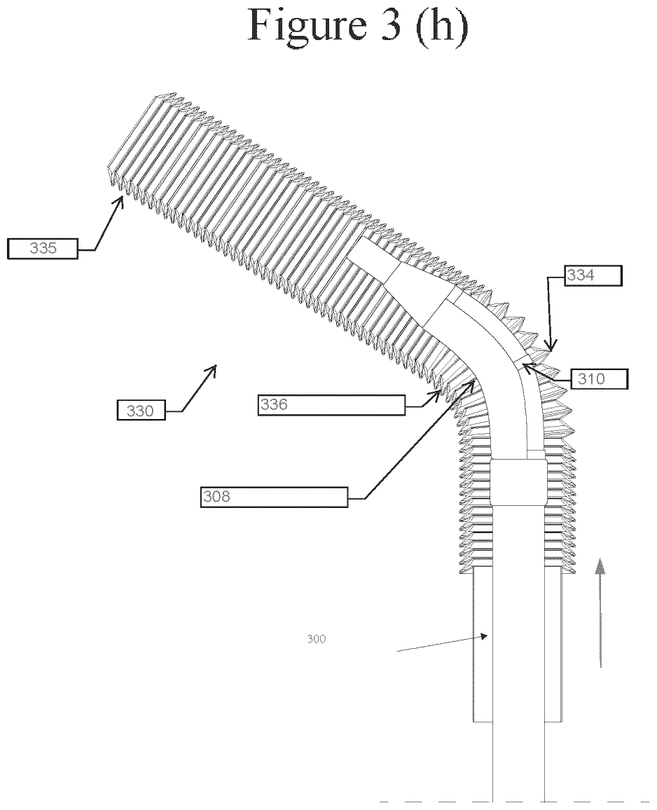

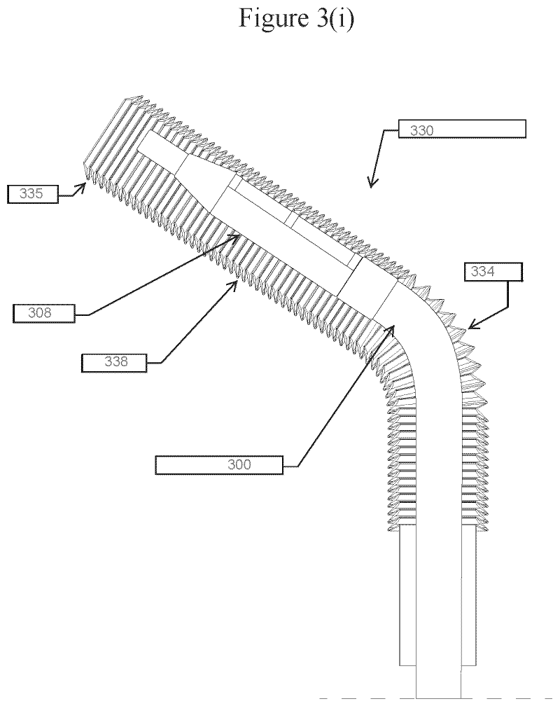

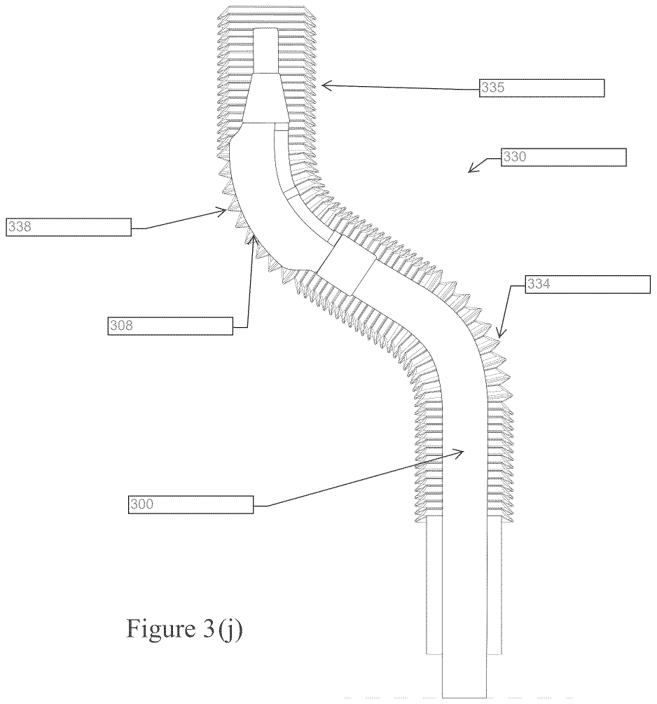

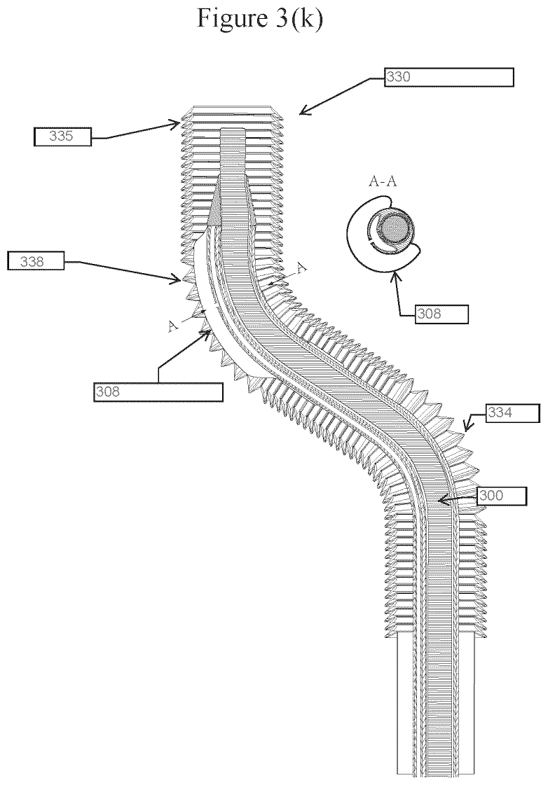

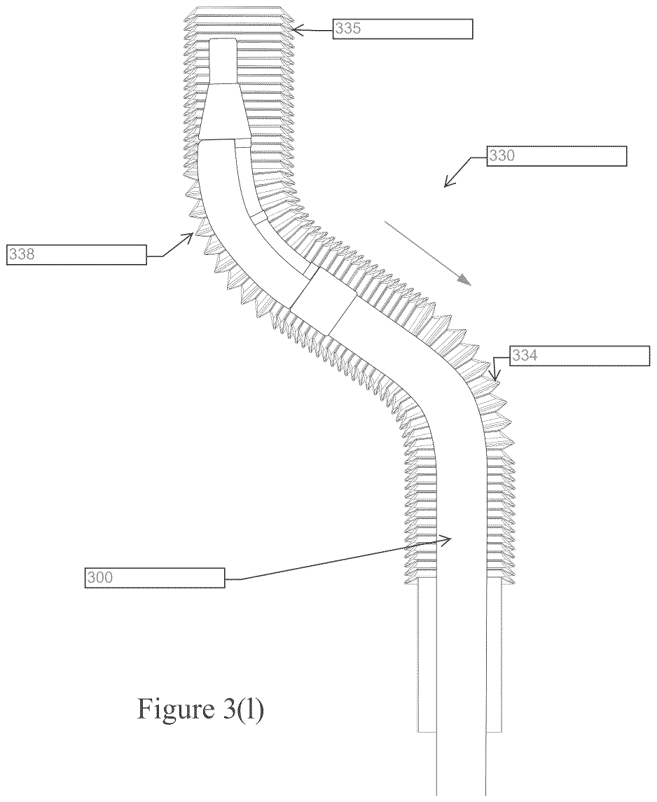

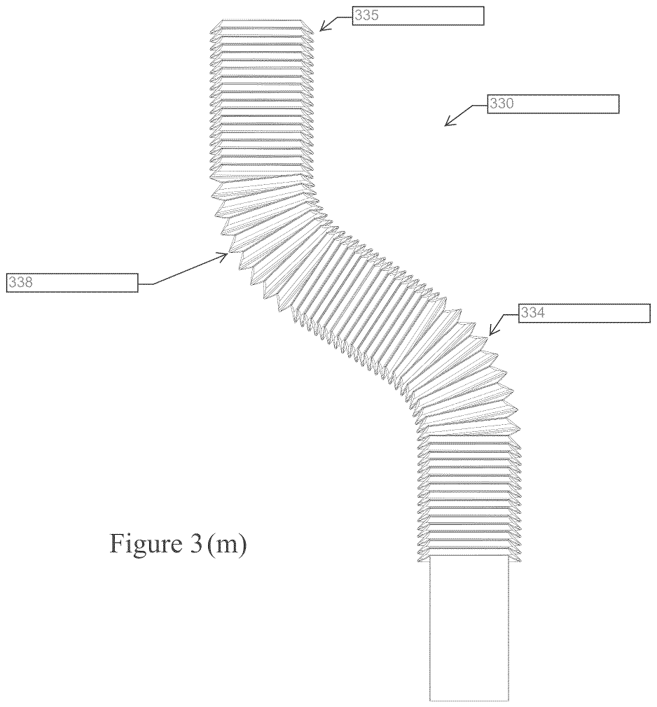

FIG. 3(a)—side-view of a double lumen (OTW) shaper 300, in accordance with some embodiments of the invention. An optional wire 302 is seen protruding from a tip 304 of shaper 300.

Exemplary Hydraulic Device Construction (See FIG. 3(a))

The hydraulic device is optionally identical to that used in FIG. 1.

FIG. 3(b)—follows FIG. 1(b).

FIGS. 3(c)-3(f)—shaper micro-catheter 300 is placed at a first curve location 334 (proximally) where a curve is desired, in accordance with some embodiments of the invention; the process of shaping the curve at this location is optionally identical to that shown in FIGS. 1(e)-1(j).

FIG. 3(g)—radial cross-section view of CFT 330, 2D side-view of shaper MC 300. A twist arrow at the base of CFT 330 shows an axial rotation of shaper MC 300.

FIG. 3(h)—radial cross-section view of CFT 330, 2D side-view of shaper MC 300. Shaper MC 300 has been rotated 180 deg. Hydraulic device membrane element 308 now lies in on a concave face 336 of the bend, whereas the optional radio-opaque markers 310 now optionally lie in the convex plane of the tip of shaper 300. The arrow at the bottom of shaper 300 shows the direction in which the shaper may be advanced.

FIG. 3(i)—radial cross-section view of CFT 330, 2D side-view of shaper MC 300. Shaper MC 300 has been advanced to a second distal location 338 where a curve is desired, in accordance with some embodiments of the invention.

FIGS. 3(j)-3(l)—the process of shaping the curve at location 338 may be identical to FIGS. 1 (e)-(j).

FIG. 3(m)—The figure shows CFT 330 in its new stable state, after a double curve has been shaped through it. The curves optionally have the same opposite curvatures, so that distal end 335 of the CFT is aligned with the axis of the proximal end of the CFT. By the same process any arbitrary 3D curve (e.g., with more or fewer curve sections, of same or different radii and/or same or different distance between curve locations) can be shaped in the CFT.

### FIG. (Set) 4—Constrained CFT Sheath/Catheter and OTW (Double Lumen) High Extension Ratio Shaper Micro-Catheter-Integral Shaping of a (Complex) Curve in the CFT

This figure set shows the process of shaping a predesigned curve in a constrained CFT—e.g., type 3 (table 2) with an integral shaper micro-catheter-type 1 (table 1), in accordance with some embodiments of the invention. When applying a rising pressure signal to hydraulic device 406, axial strain may follow in a membrane 408 (e.g., directed from proximal end to distal end) and gradually advance membrane 408 (e.g., a balloon) through CFT 430. In some embodiments of the invention, with the initial pressure rise sufficient radial deformation is established in membrane 408 which will be maintained throughout its advancement. Optionally, gradual axial strain will actuate CFT 430 cell by cell, and when a constrained segment is encountered the CFT will curve locally according to the constraint, while straining open all parts of the cell which are not constrained. Optionally, CFT 430 is configured with a higher rigidity than membrane 408 so that membrane 408 follows the created curvature while actuating CFT 430.

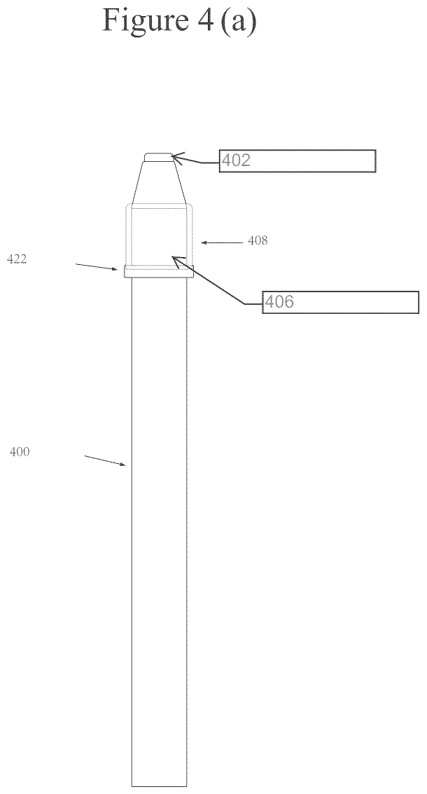

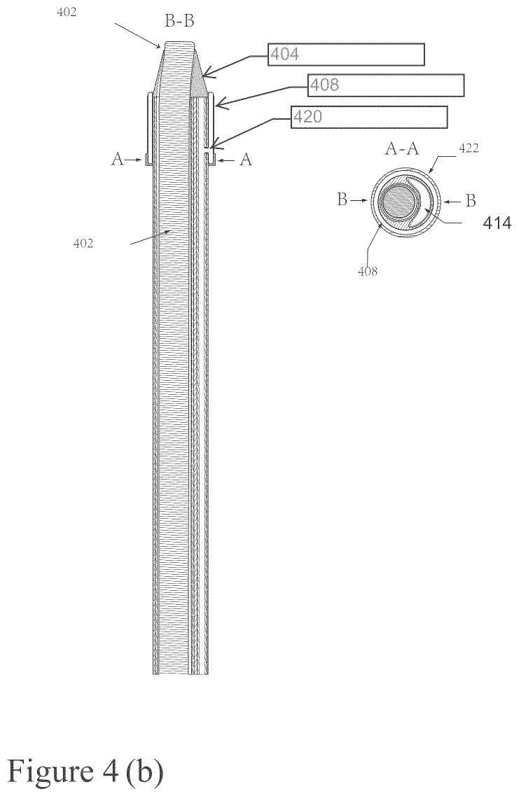

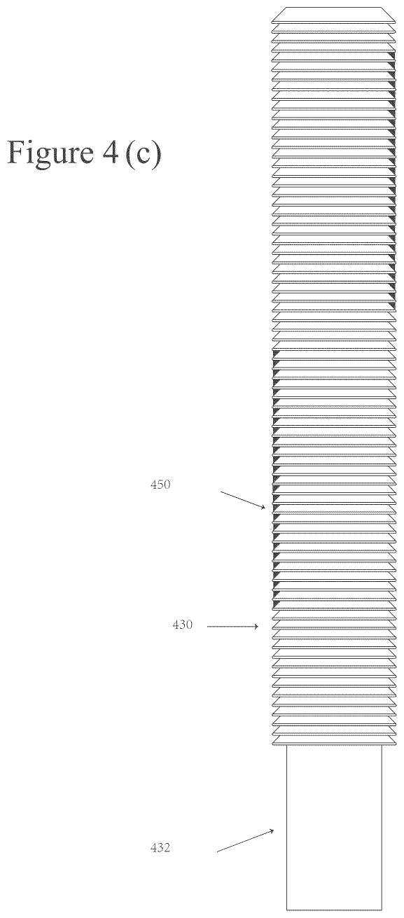

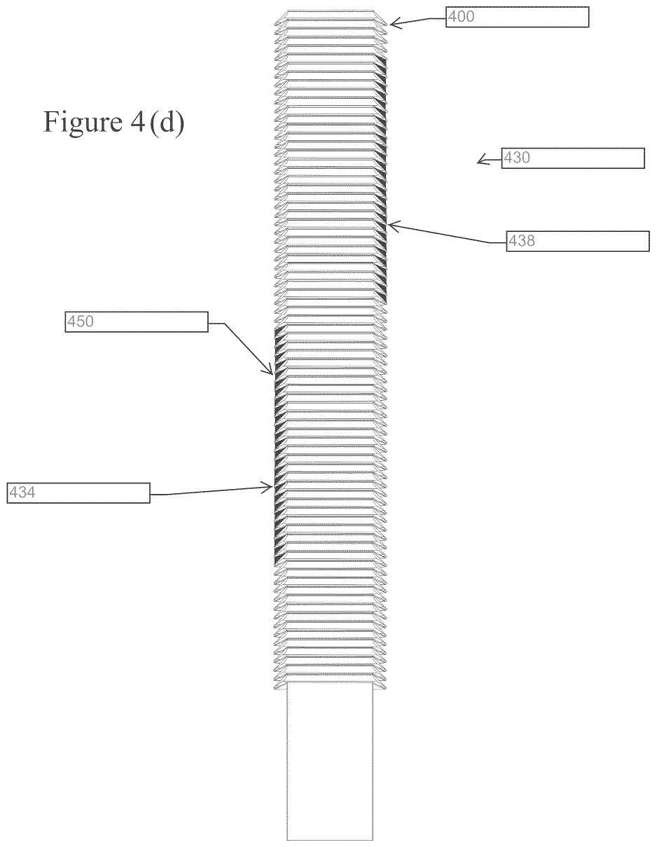

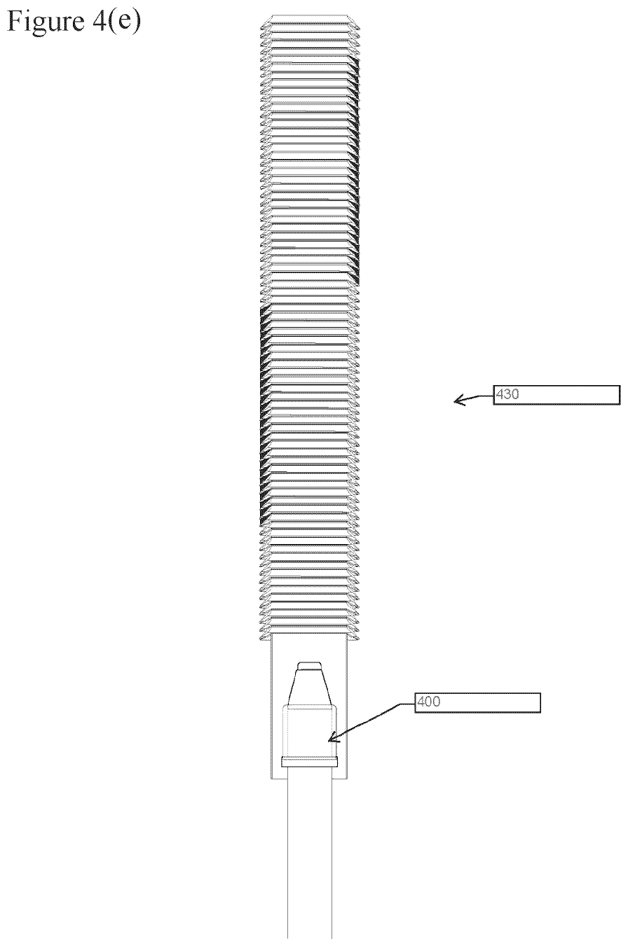

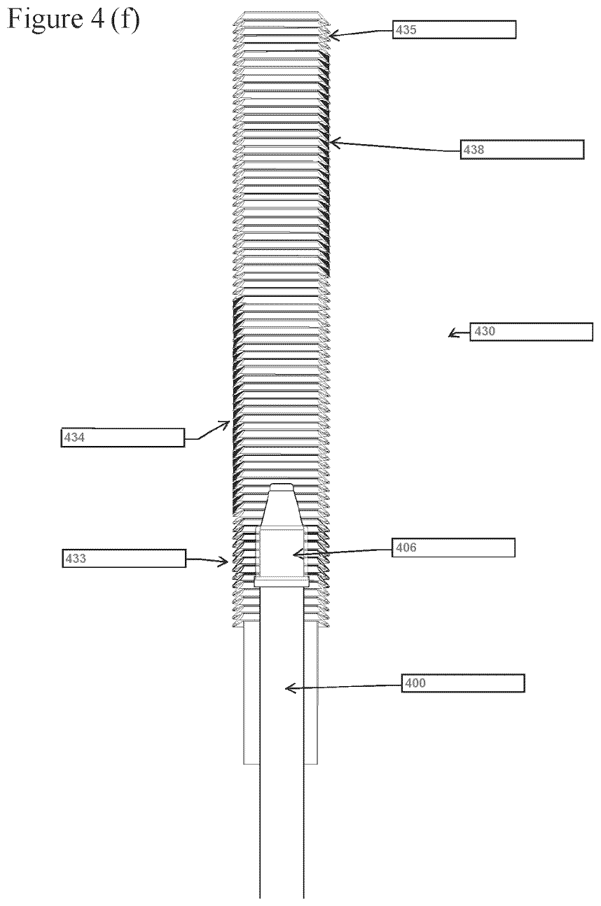

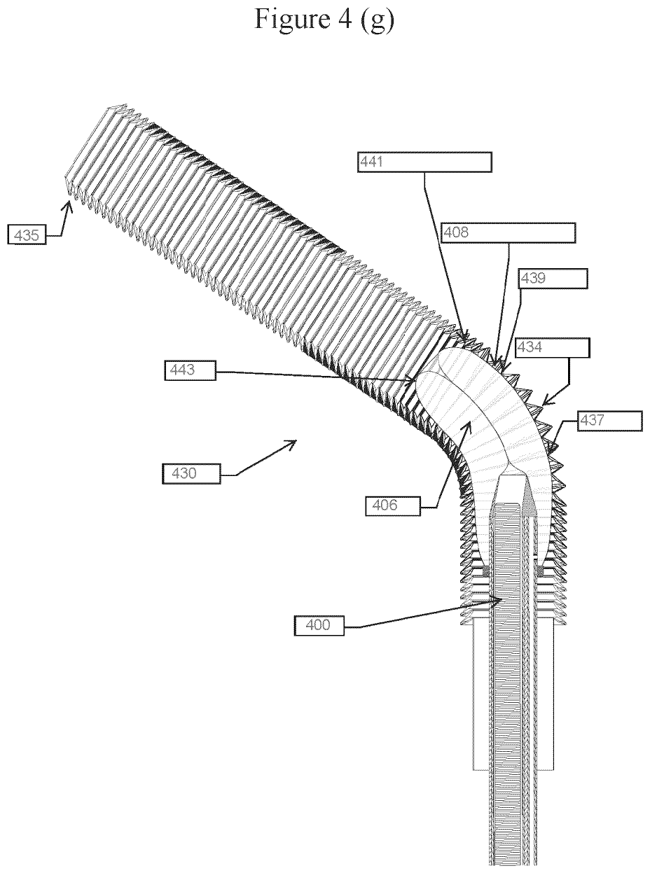

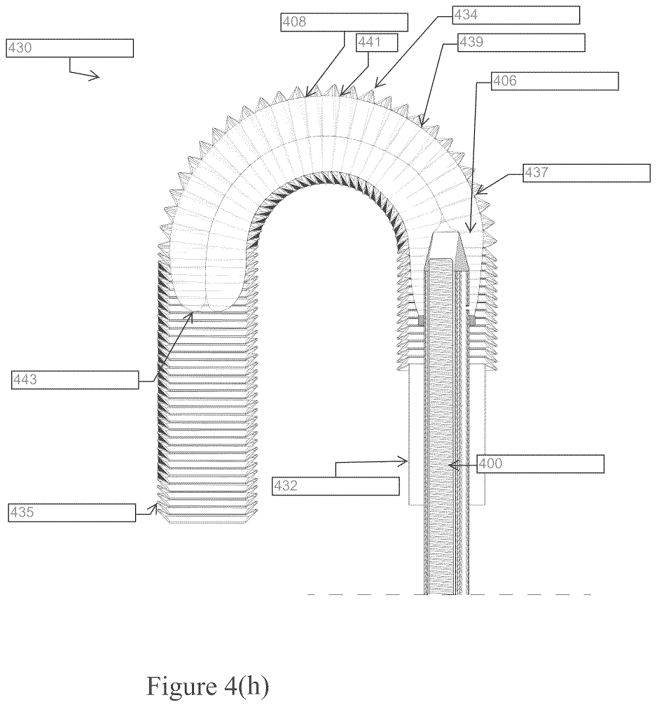

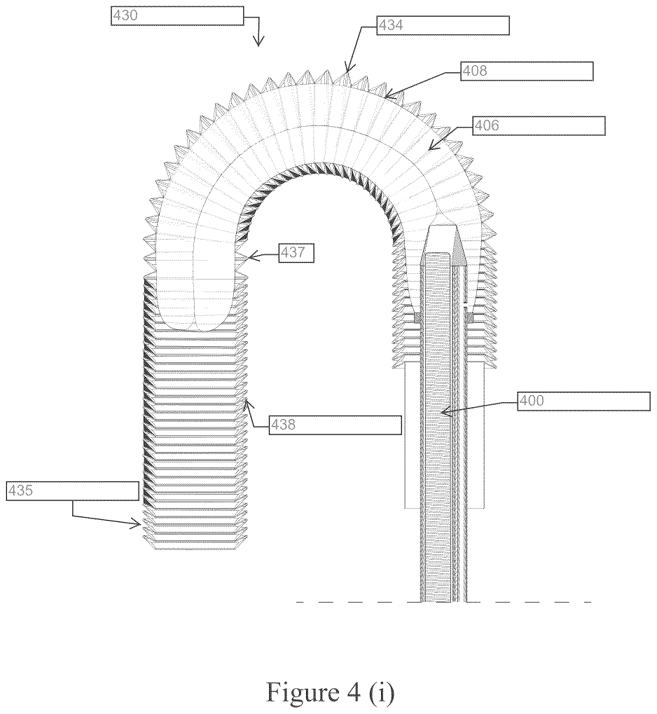

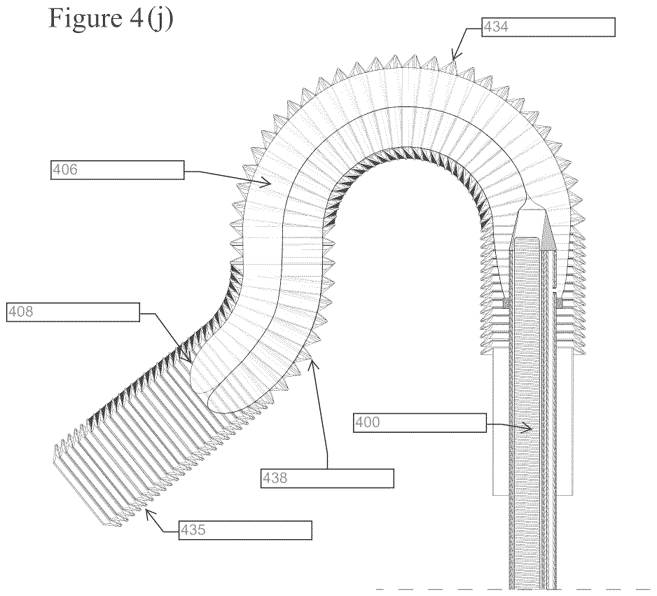

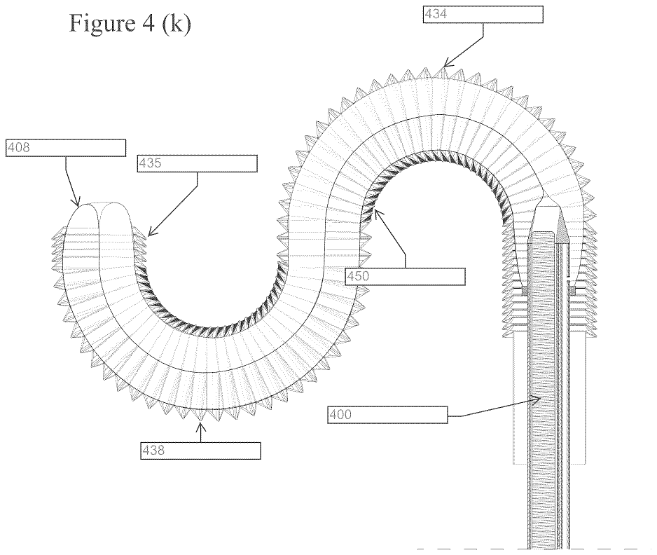

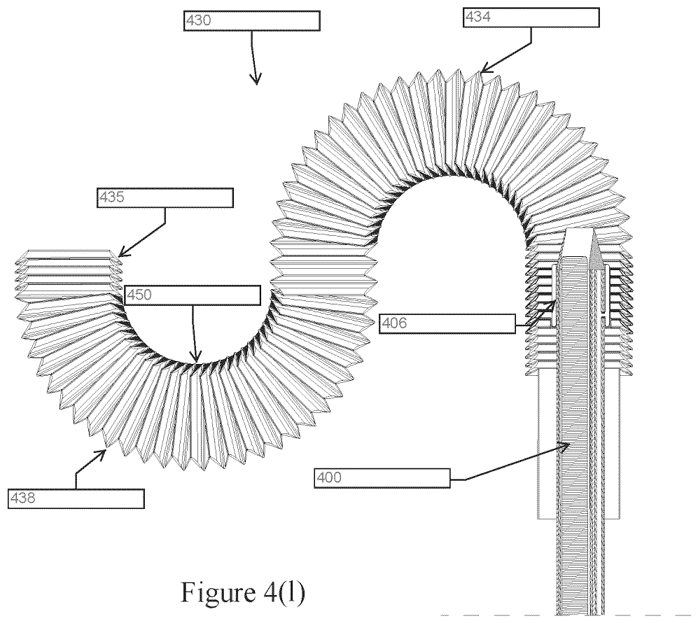

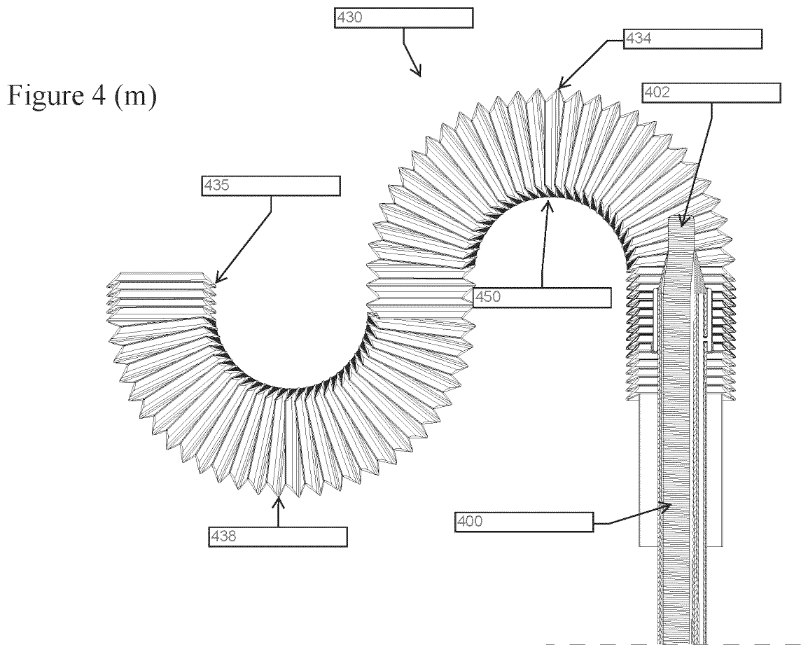

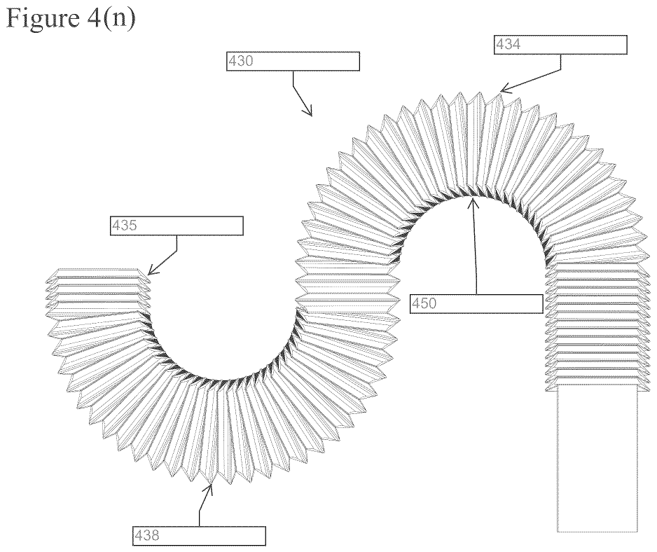

FIG. 4(a)—side-view of double lumen (OTW) shaper 400, in accordance with some embodiments of the invention. An optional wire 402 is seen protruding the tip of the shaper. Hydraulic extension device 406 at the distal end thereof optionally has a torus cross-section and is made of an elastic membrane designed to form large extensions of CFT 430 by latching on to its internal wall while distancing any two material points away from each other. The strain field is optionally formed uniformly and/or axi-symmetrically along the circumference of a membrane 408 thereof, thereby potentially extending the CFT correspondingly. In some embodiments of the invention, CFT 430 has locked (or otherwise constrained) cells (e.g., type 3)—the locking takes effect along an axial line with a predefined locking azimuth (θ(z), where θ is the azimuthal angle and z is the axial coordinate), FIG. 4(c) shows a straight and two-dimensional locking line 450 which may be used to provide two-dimensional deflection. Locking is optionally implemented inherently in the manufacturing process of CFT 430.

Exemplary Hydraulic Device Construction (See FIG. 4(b))

In some embodiments of the invention, Hydraulic mechanism 406 includes an extensible torus membrane 408; it remains secured at a tip 406 of shaper 400, below the tapered cross-section segment, when unactuated. The torus shape optionally enables the stable passage of a guidewire 402, especially when the mechanism has been curved through CFT 430. In some embodiments of the invention, torus membrane 408 is stretched axi-symmetrically around the micro-catheter, and when pressurized it optionally undergoes a combination of radial strain, which may provide the latching force onto the inner wall of CFT 430, and/or axial strain which may provide the extension force for CFT 430. In some embodiments of the invention, device 406 is configured to undergo significant axial extension up to tenfold its unactuated length.

FIG. 4(b)—radial cross-section of shaper micro-catheter 400. The figure shows the catheter construction and hydraulic device 406 construction. An axial cross-section view of the exemplary hydraulic device is also viewed in the figure (A-A) including two visible lumens: a wire lumen and an inflation lumen 414, an inflation orifice 420 suppling pressurized saline to elastic membrane torus 408.

FIG. 4(c)—side view of CFT 430—unactuated state, in accordance with some embodiments of the invention. Its inner diameter can be seen based on a proximally extending elongate shaft section 432 with no CF sections. The steering mechanism is optionally completely independent of the construction of shaft 432 and the properties of shaft 432 can be optionally optimized independently of the function of CFT 432. Two subsequent locked cell series' are implemented in this example of CFT 430, having 27 locked cells each. Other numbers may be provided as well. Optionally, the locked segments alternate 180 degrees in the azimuth of their locking lines which are straight and in-plane in this example. This design may be used to provide the dynamic shaping of a planar S curve (proximal to distal progression).

It is noted that shaping a double curve has a potential advantage because one of the major challenges in conventional guide-catheters is to curve a distal curve in an opposite direction of the already existing traction of the proximal curve. When pushing from the hub, the catheter always wants to go along the proximal curve or other existing proximal traction. In many cases of tortuous anatomies (e.g. abdominal) there is a need for controlling a double curve—i.e. change the traction along the tip in an opposing direction and even more importantly in 3D.

In addition, the CFT can optionally be designed to provide conformal progression along the vasculature so that no anchorings against artery walls are required, which potentially reduces the risk of perforations and/or arrhythmia and/or sliding along tissue. Such design can also provide a combination of curved sections and shaft extended sections, so that a wide range of anatomies may be navigated. Shaft extended sections can be used to extend the distal shaft into the anatomy after a turn has been made so that further anchoring can be achieved when hub forces cannot be transferred along the traction of the tip. This, for example, may be useful in a guide-catheter setting when a curve has been established but any transfer of subsequent devices (e.g. micro-catheter, balloon catheter or wire) alters the traction along the curve pushing the guide-catheter out of position. There are many relevant anatomies in which these situations may occur, including (but not limited to):
peripheral: abdominal aorta to renal placement, abdominal aorta to celiac trunk, splenic artery navigation;
coronary: any type of severe vessel tortuosity or angulation and specifically extreme angulation of the side-branch in relation to the main vessel or severe stenosis in both main vessel ad side-branch.

FIG. 4(d)—radial cross section view of CFT 430 (radial cross-section of FIG. 4(c)).

FIG. 4(e)—radial cross-section view of CFT 430 along with a side-view of shaper micro-catheter 400 inserted over a guidewire 402 and positioned distally prior to its advancement into CFT 430.

FIG. 4(f)—radial cross-section view of CFT 430 along with a side-view of shaper micro-catheter 400 inserted over guidewire 400 and advanced to an actuation position 433, optionally at a first bend location 434.

FIG. 4(g)—radial cross-section view of CFT 430 along with a radial cross-section view of shaper micro-catheter 400 inserted over guidewire 402. Hydraulic device 406 has been actuated, in accordance with some embodiments of the invention and advanced into first locked cells segment 434. As shown, the first 10 cells have been curved at this point. The actuation and advancement of hydraulic device 406 can be done over a soft (e.g. tapered) portion of the guidewire so that it doesn't resist the curve. It is noted that where activated over unconstrained cells, in some embodiments of the invention, an axial extension may be expected. Also, while the deployment is shown as starting at a proximal position 433, one may start at a distal position and retract device 406 proximally and/or design membrane 408 so it expands proximally (in addition to or instead of extending distally, as shown).

FIG. 4(h)—radial cross-section view of CFT 430 along with a radial cross-section view of shaper micro-catheter 400 inserted over a guidewire. The hydraulic device has been advanced over first locked cells segment 434 and has curved it 180 degrees in the opposite direction of proximal shaft 432.

By comparing FIGS. 4(g) and 4(h), the mechanism of action of membrane 408 may be visualized. This mechanism applies to other embodiments herein with an axially expanding membrane. Starting with FIG. 4(g). When membrane 408 first expands, it expands radially so that points 437 and 439 (on the inner surface of the lumen of CFT 430) are frictionally and/or geometrically engaged. Point 441, further axially along CFT 430 is not reached or engaged. As membrane 408 is further expanded, it is constrained from radial expansion by CFT 430 and must expand axially. This example is both at a tip 443 thereof and at membrane portions contacting the wall, such as the section between points 437 and 439. The axial expansion of this membrane section causes points 437 and 439 to be moved apart, thereby causing a bend, as shown. The axial expansion at 443 causes the radial engagement of membrane 408 to extend axially along CFT 430, for example, to engage point 441 (see FIG. 4(h)). Further expansion will, again, cause separation between point 439 and 441, causing more CFT-cells to be expanded, resulting, in this example, in more bending. Bending between points 437 and 439 may be constrained by the limits of CFT deformation.

In some embodiments of the invention, membrane 408 is in the form of a thin walled balloon, for example, one with a diameter smaller than the CFT inner diameter (e.g., so when collapsing does not apply force on wall). Optionally the wall includes one or more protrusions or serrations for better engagement and/or matching with the inner wall of CFT 430. Optionally, the diameter is not too small, so as to allow enough expandability for axial expansion and/or to reduce axial expansion which occurs before radial expansion is completed. Optionally, membrane 408 is designed to expand at least by a factor of 3, 5, 7, 10, 15, 20 or greater or intermediate amounts.

In some embodiments of the invention, membrane 408 has non-linear expansion properties, for example, expanding radially more easily than axially. For example, by including one or more axial threads which are more resilient than the body of membrane 408. Optionally, different axial locations along membrane 408 have different expansion properties, for example, to control the relative amount of axial extension in, for example between axially spaced apart points 437, 439 and 441.

FIG. 4(i)—radial cross-section view of CFT 430 along with a radial cross-section view of shaper micro-catheter 400 inserted over guidewire 402. Hydraulic device 406 has been advanced further into intermediate cells 437 which are located in between the locked cell segments at 434 and 438. CFT 430 has been optionally extended axially in this region and hydraulic device 406 has reached the second locked cell segment 438 but has not curved it yet.

FIG. 4(j)—radial cross-section view of CFT 430 along with a radial cross-section view of shaper micro-catheter 400 inserted over guidewire 402. Hydraulic device 406 has been advanced into second locked cell segment 438 and has begun to curve it, in accordance with some embodiments of the invention.

FIG. 4(k)—radial cross-section view of CFT 430 along with a radial cross-section view of shaper micro-catheter 400 inserted over guidewire 402. Hydraulic device 406 has been advanced throughout second locked cell segment 438 curving it 180 degrees, completing the formation of the S shaped planar curve, in accordance with some embodiments of the invention.

FIG. 4(l)—radial cross-section view of CFT 430 along with a radial cross-section view of shaper micro-catheter 400 inserted over guidewire 402. Hydraulic device 406 has been retracted from CFT 430 optionally back to its original unactuated form. CFT 430 remains stable holding the predesigned double curve, in accordance with some embodiments of the invention.

FIG. 4(m)—radial cross-section view of CFT 430 along with a radial cross-section view of shaper micro-catheter 400 inserted over guidewire 402, in accordance with some embodiments of the invention. Following FIG. 4(l), guidewire 402 is shown to optionally advance into the newly shaped double curve. The guidewire may be longer than shown, for example, extending out of end 435.

FIG. 4(n)—radial cross-section view of CFT 430 along with a radial cross-section view of shaper micro-catheter 400 inserted over guidewire 402. Assuming the target anatomy has been reached, shaper micro-catheter 400 has been retraced out of the catheter/sheath including CFT 430 for optional subsequent treatment via such catheter/sheath.

FIG. (Set) 5—CFT Sheath/Catheter and OTW (Double Lumen) Flexural Shaper Micro-Catheter, with Intrinsic Curvature and Punctured Sphere Like Geometry at the Tip, Purpose—Shaping a Curve in the CFT

The figure set shows the process of shaping a two-dimensional curve in a CFT 530 by a double lumen micro-catheter shaping device 400 (shaper of type 5—see table 1 and CFT of type 1—see table 2), in accordance with some embodiments of the invention. The shapers in this figure set optionally have the same design as the shapers described in FIG. 1 except they optionally have one or more additional features that may improve their crossing ability in consideration of a possibly non-smooth surface of CFT 530—at the bend scale and/or at the individual cell scale, as follow: The shaper is fabricated with intrinsic curvature, in the figure optionally such curvature geometry is presented, which is a curvature resembling a “Cobra” angiographic catheter shape. When it is required to move it forward through straight segment in CFT 530 its elasticity enables it to adopt a straight shape and slide forward. When it is required to move it forward through a bent segment in CFT 530 its intrinsic curvature help it slip forward along the CFT. Another feature that may improve crossing ability of the shaper is a punctured sphere or other rounded geometry, made from rigid or semi rigid material that is fabricated at a tip 504 thereof, at a more distal location from asymmetrically expanding membrane 508 (tip 504 hereafter termed “punctured bulb”). These features, which are designed to improve the crossing ability through a non-smooth CFT are demonstrated for a flexural shaper, but are relevant and can be fabricated on a shaper of any of the other figures and types, including extension, in addition or instead.

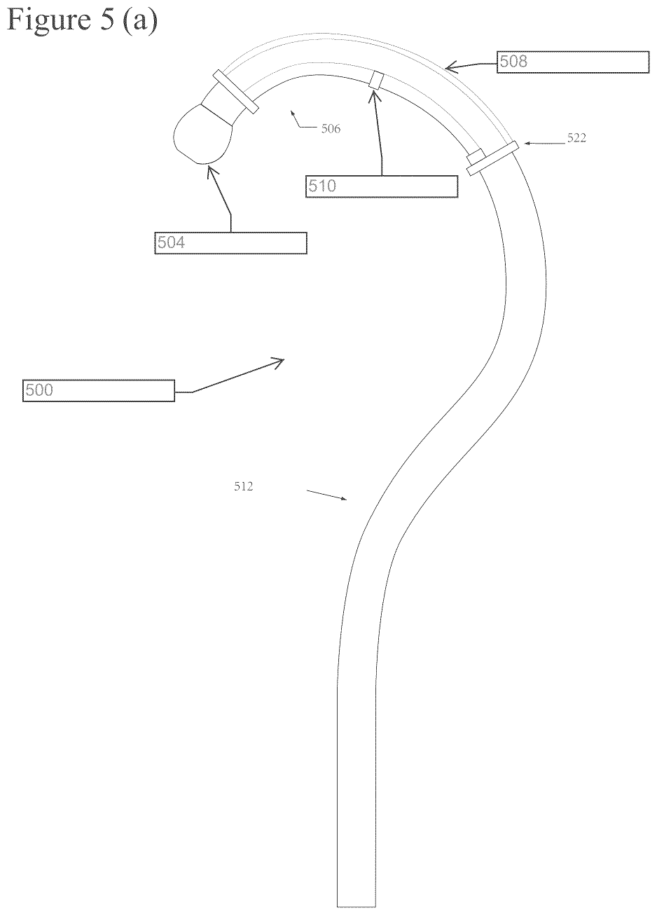

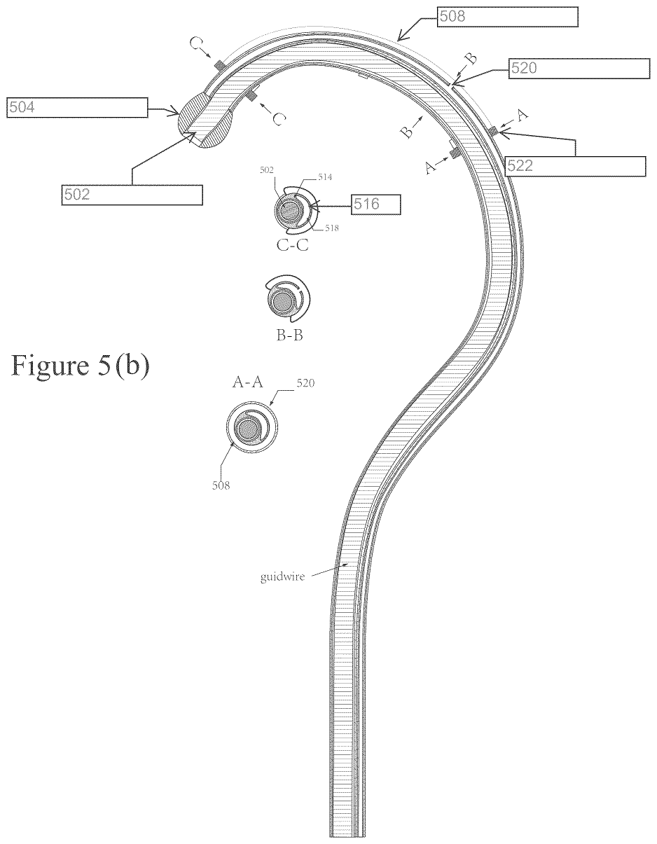

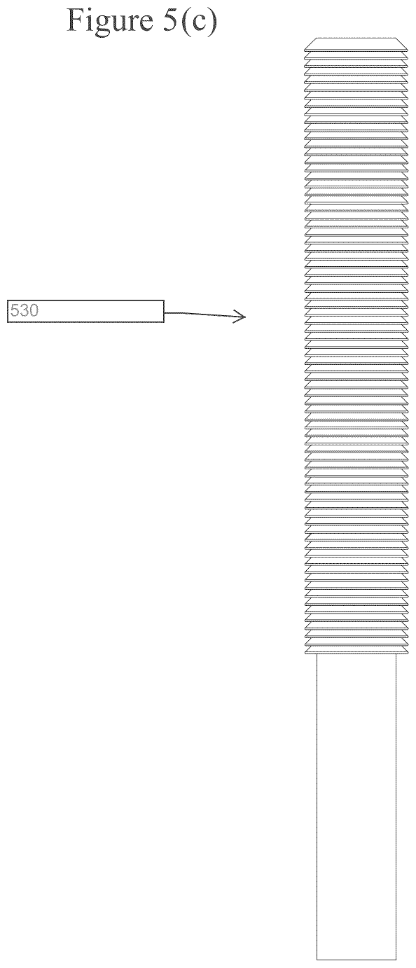

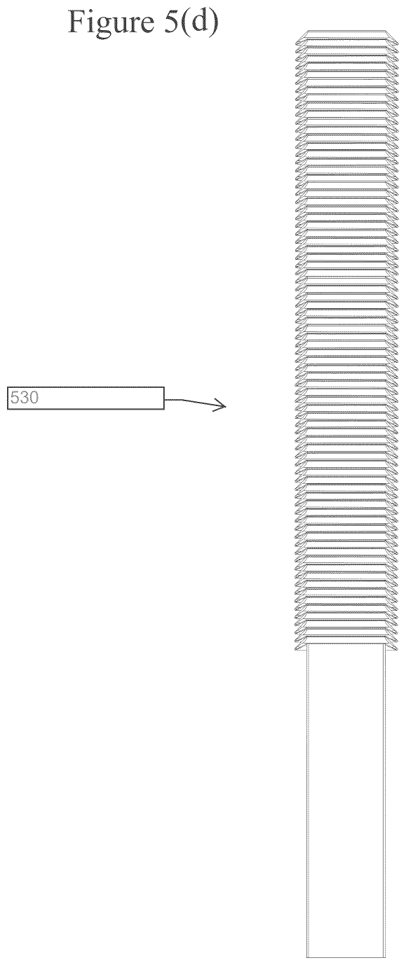

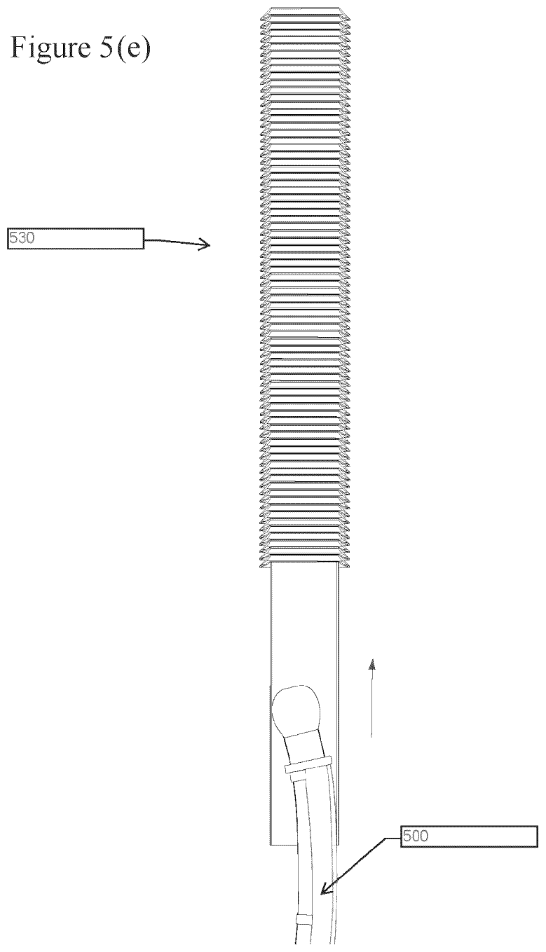

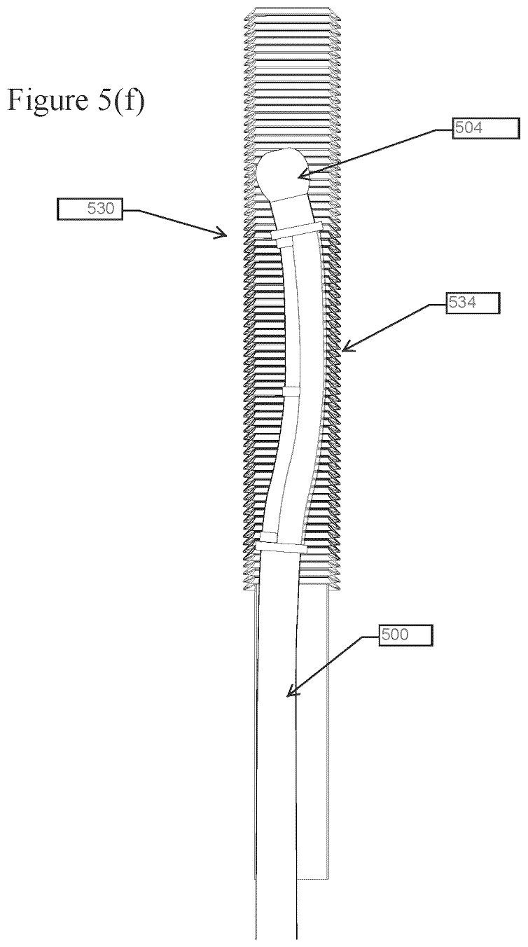

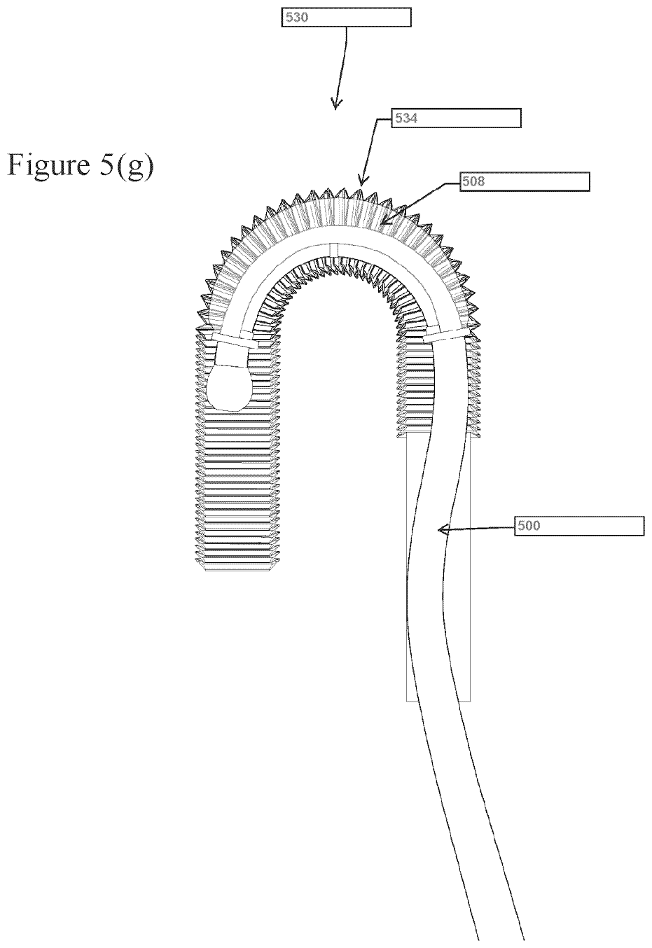

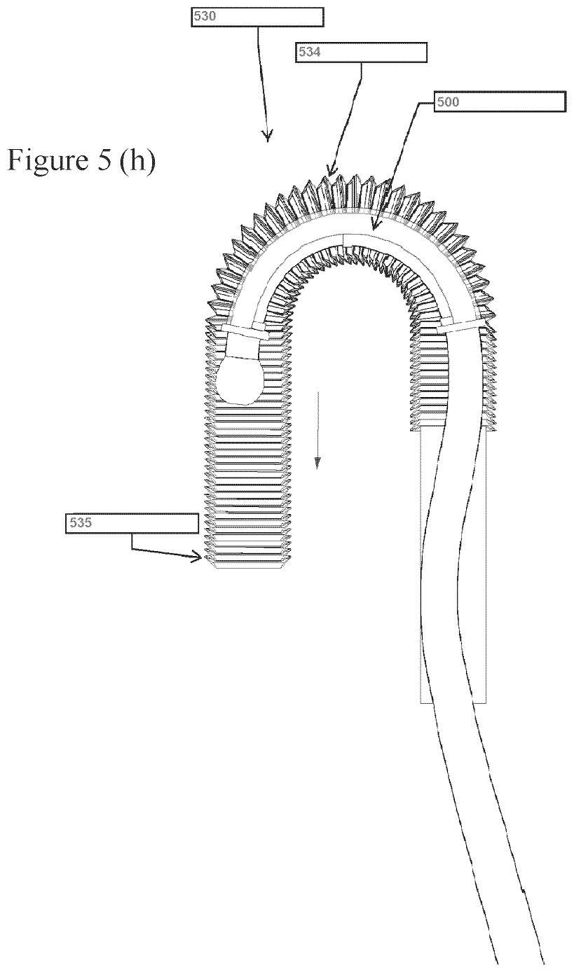

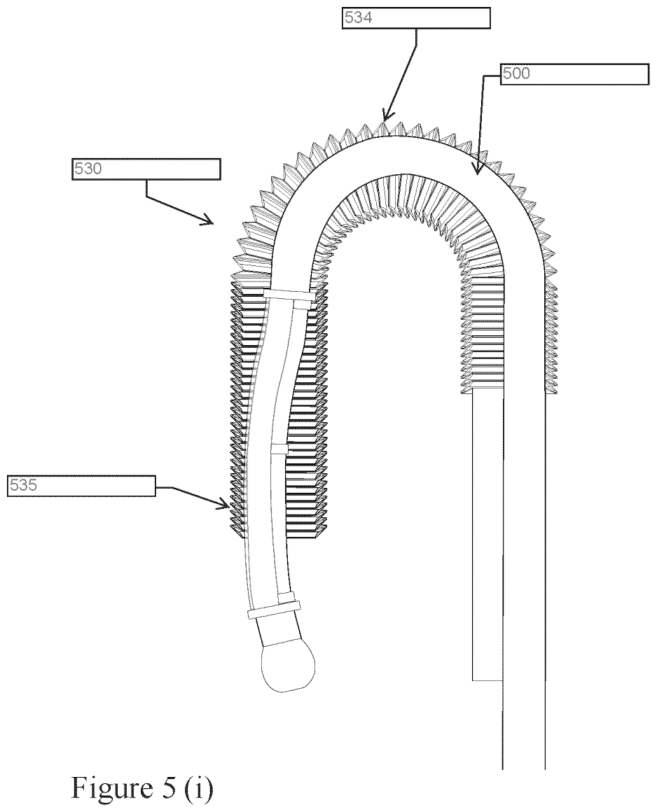

FIG. 5(a)—an exemplary two-dimensional side-view of a double lumen (OTW) shaper 500. The intrinsic curvature and punctured bulb at tip 504 can be seen. A guidewire 502 can cross along shaper 500 and through punctured bulb 504.

### Exemplary Hydraulic Device Construction (See FIG. 5a))

Hydraulic device 506 optionally includes an inflated elastic cylindrical tube or membrane 508 (hereafter termed membrane) and is optionally designed to form a curve in CFT 530. Such curving is optionally provided by latching (e.g., using a friction mechanism) radially asymmetrically on to the internal wall of CFT 530 while distancing two material points away from each other. When membrane 508 is pressurized, a strain field begins to forms optionally almost exclusively in the surface being designed to yield first (optionally membrane 508 is fabricated to include areas with thinner thickness to yield first and areas with thicker thickness to yield last (the face of the membrane designed to yield first hereafter termed “convex face”, the opposite side hereafter termed “concave face”), the membrane inflates and touches the internal wall of CFT 530, and the mechanism of curving CFT 530 operates. The design optionally includes one or more radio-opaque markers 510 on the concave face of membrane 508 which can provide imaging feedback as to the location of shaper 500 along CFT 530 and/or as to the curvature of membrane 508.

FIG. 5(b)—Radial cross-section view of shaper micro-catheter 500 with two visible lumens, in accordance with some embodiments of the invention: wire lumen 514 and inflation lumen 516. An inflation orifice 520 supplies pressurized saline to membrane 508, optionally elastic. An axial cross-sectional view of the shaper construction is also viewed to the right of the axial cross-section describing a top cross-sectional view of the above.

FIG. 5(c)—two-dimensional side view (radial) of CFT 530 in an unactuated state, in accordance with some embodiments of the invention. The CFT shown is a particular case of the CFT geometry. Its extension ratio is, for example, 1:2.25.

FIG. 5(d)—radial cross-section view of CFT 530.

FIG. 5(e)—radial cross-section view of CFT 530 along with a two-dimensional side-view (radial) of shaper micro-catheter 500 positioned at the proximal end of CFT 530.

FIG. 5(f)—radial cross-section view of CFT 530 along with a two-dimensional side-view (radial) of shaper micro-catheter 500 as it is advanced axially into CFT 530 and positioned at a desired location 534 where shaping of CFT 530 is desired, in accordance with some embodiments of the invention. In this and other embodiments, “at a desired location” refers generally to an alignment of the active area and/or reach of membrane 508 with regard to a portion of CFT 530 that is to be deformed (and which can be deformed). In some embodiments of the invention, what is aligned is an axial center of the membrane (e.g., as an example of a point of maximal deformation).

In some embodiments of the invention, membrane 508 is axially short and a curve is created by deforming first section of CFT 530 and then moving shaper 500 to a nearby location for further deformation. This can allow a curve with variable curvature to be created. For example, a shaper be sized to engage fewer than 50, fewer than 30, fewer than 20, fewer than 10, fewer than 5 and/or more than 2 CFT cells at a time. FIG. 11 shows an opposite case where many CFT cells are engaged with a single balloon.

It can be seen how shaper's 500 intrinsic curvature adopts straighter shape in accordance with the straight alignment of CFT 530, and punctured spherical tip 504 can be thought as sliding along the zigzag inner surface of CFT 530 as the shaper advances axially. In some embodiments of the invention, shaper 500 is made soft enough so that it applies (when not hydraulically activated) lower forces than needed to deform CFT elements of CFT 530.

FIG. 5(g)—radial cross-section view of CFT 530 along with a two-dimensional side-view (radial) of shaper micro-catheter 500 as it is actuated hydraulically, in accordance with some embodiments of the invention. Upon actuation, CFT 530 is optionally curved about the center of elastic membrane 508.

FIG. 5(h)—radial cross-section view of CFT 530 along with a two-dimensional side-view (radial) of shaper micro-catheter 500 as it is hydraulically deflated, in accordance with some embodiments of the invention, optionally as preparation step for another axially advancement along CFT 530.

FIG. 5(i)—radial cross-section view of CFT 530 along with a two-dimensional side-view (radial) of shaper micro-catheter 500, in accordance with some embodiments of the invention as shaper 500 is advanced past bent area 534 and optionally past a distal end 535 of CFT 530. As can be seen, the intrinsic curve of shaper 500 matches to the actual curve (or lack thereof) of CFT 530, as the force applied by the non-actuated catheter is optionally not sufficient to deform any CFT cells in CFT 530.

FIG. (Set) 6—CFT Sheath/Catheter and OTW (Double Lumen) Flexural Shaper Micro-Catheter, with a Fixed Antenna Like Wire and a Sphere Like Geometry at the Tip—Shaping a Curve in the CFT

This figure set shows the process of shaping a two-dimensional curve in a CFT 630 by a double lumen micro-catheter shaping device 600 (e.g., shaper of type 5—see table 1 and CFT of type 1—see table 2), in accordance with some embodiments of the invention. The shapers in this figure set optionally have the same design as the shapers described in FIG. 1 except they may include one or more additional features that improve its crossing ability along CFT 630 and especially in consideration of the potentially non-smooth topology of an inner surface of CFT 630. At the distal tip, at a more distal location from an (optionally) asymmetrically expanding membrane 608, an elongate element 603 is provided (resembling an antenna, and can be made in the same shape and from the same material as conventional nitinol guide wires, hereafter termed “antenna”). It is potentially advantageous but not necessary for its design to be tapered. Optionally, antenna 603 is stiffer at its base than at its distal end. At the distal tip of antenna 603, a sphere or other rounded geometry 604, optionally made from rigid or semi rigid material is optionally provided (hereafter termed “bulb”). Antenna 603is optionally used for diverting the stiffer part of shaper 600, to which it is attached, in the same direction that CFT 630 curves, when shaper 600 is required to slip forward along CFT 630. Optionally, antenna 603 blocks the wire lumen, so a guidewire can extend along shaper 600 but not protrude distally from shaper 600. Such wire (or stylet) may provide shaper 600 with better kink resistance and/or better pushability. In some embodiments, such properties are not required and/or for other reasons, a guidewire and its lumen may be omitted form the design. This may allow for the production of smaller diameter shaper. The above described features, which potentially improve the crossing ability through the zigzag surface of a CFT are demonstrated in a figure of a flexural shaper, but are relevant and can be fabricated on extension shaper just the same. Also, in some embodiments of the invention, bulb 604 includes a port for a guidewire.

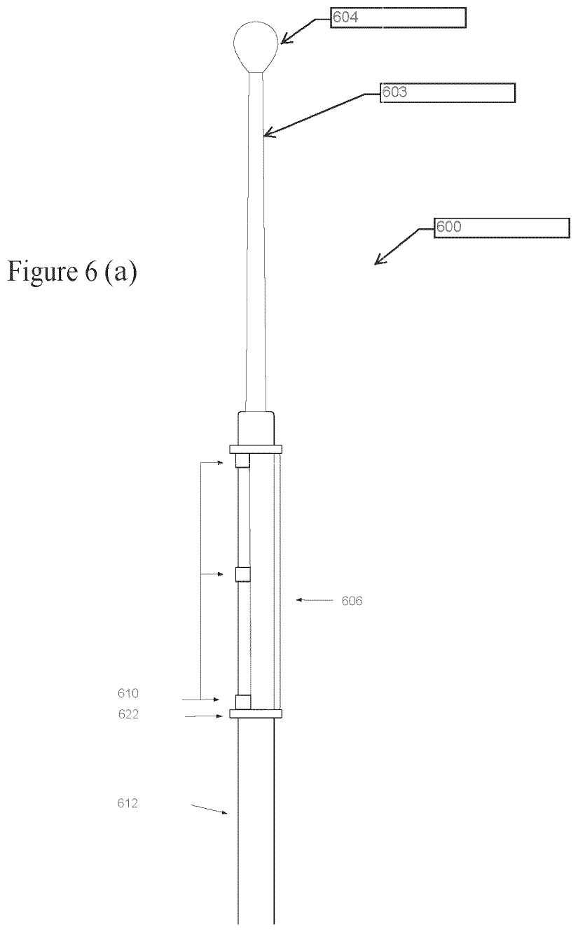

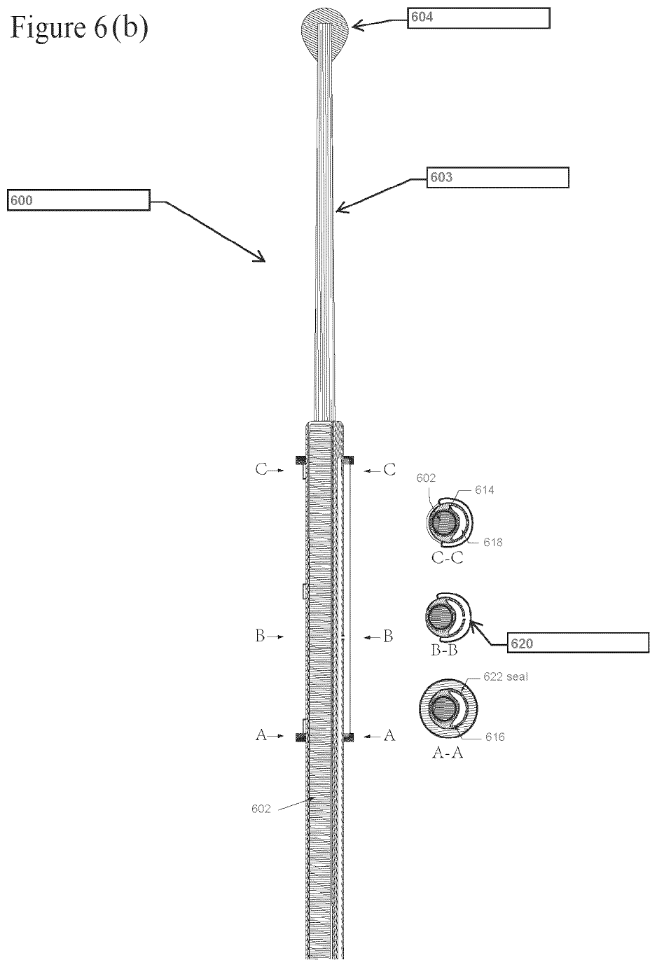

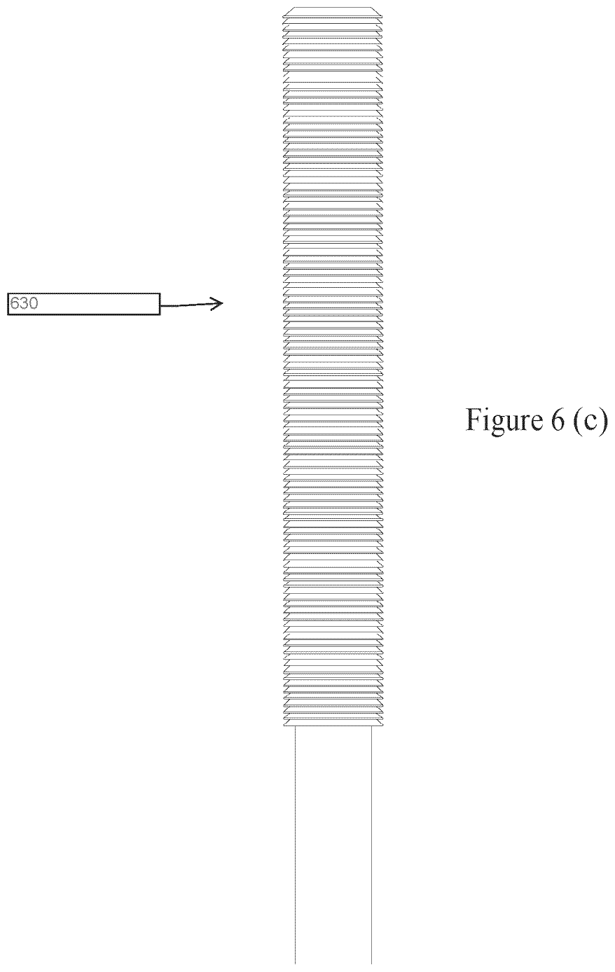

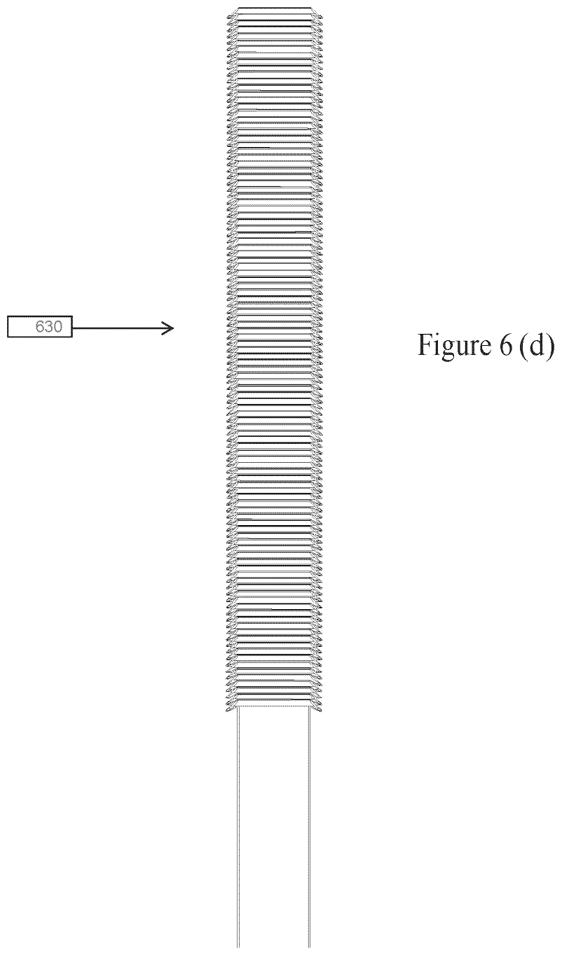

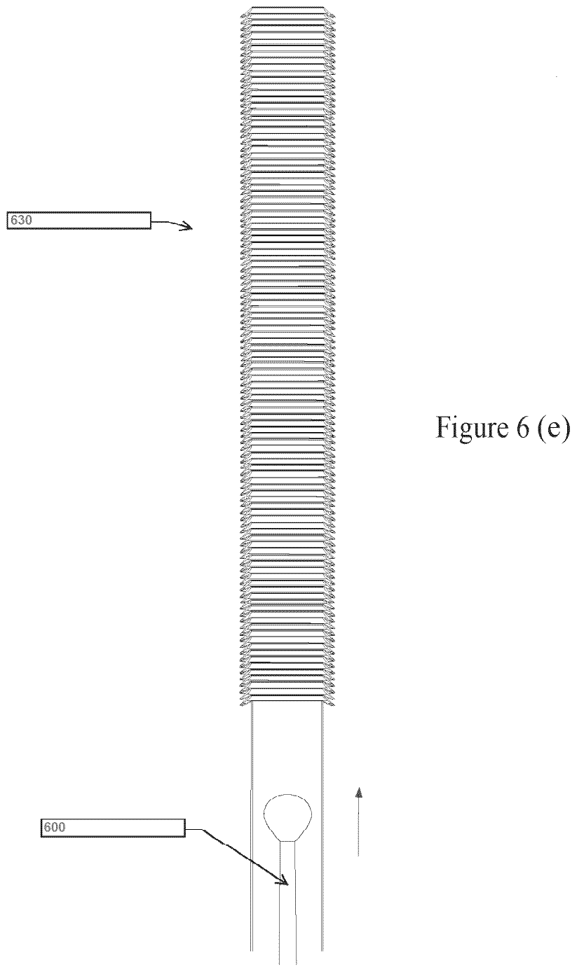

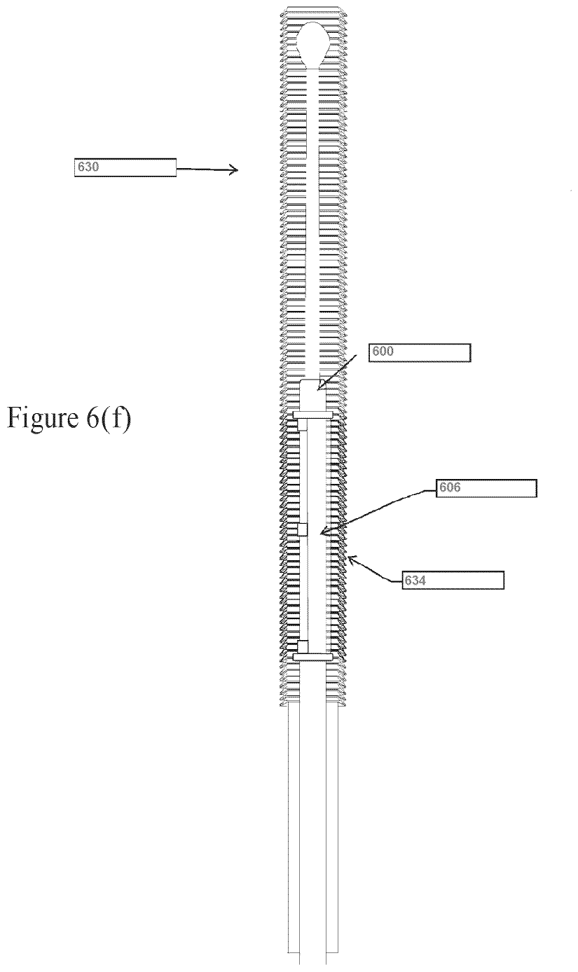

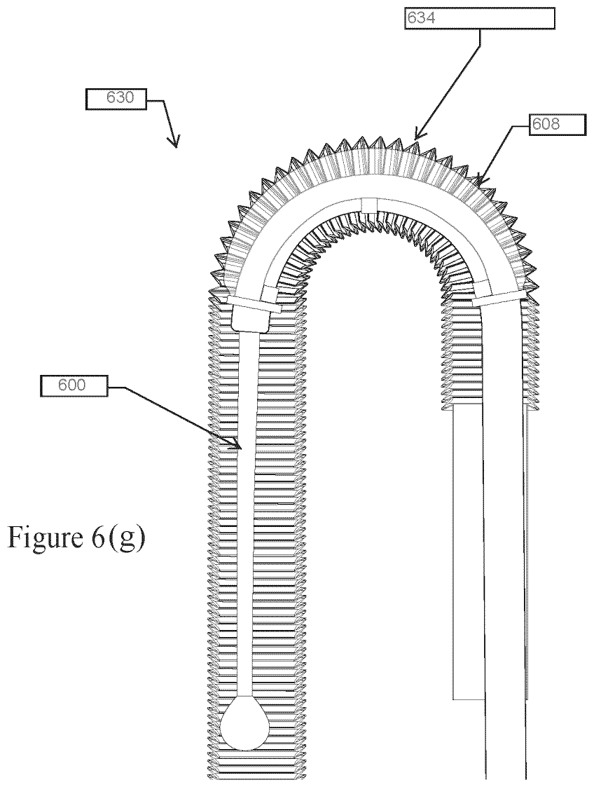

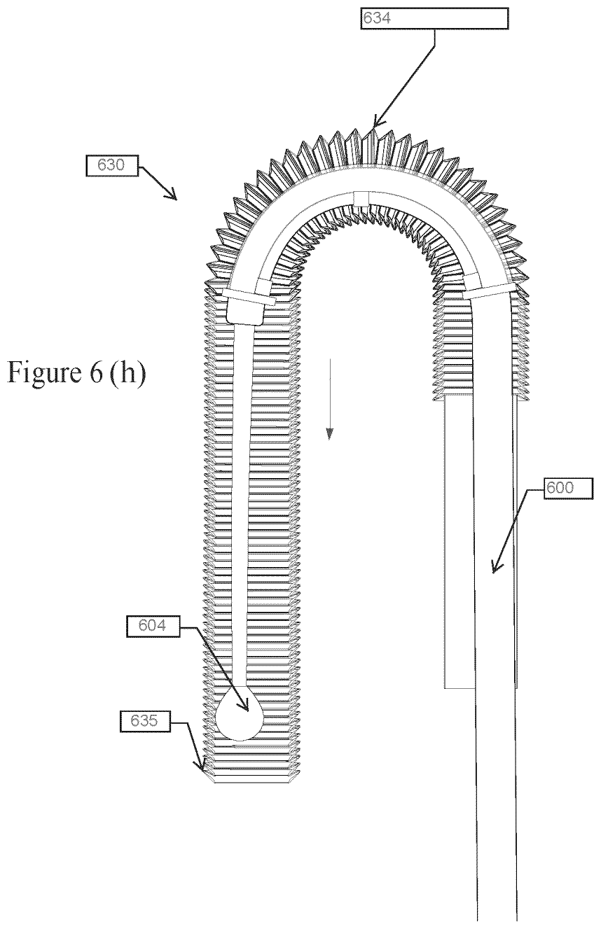

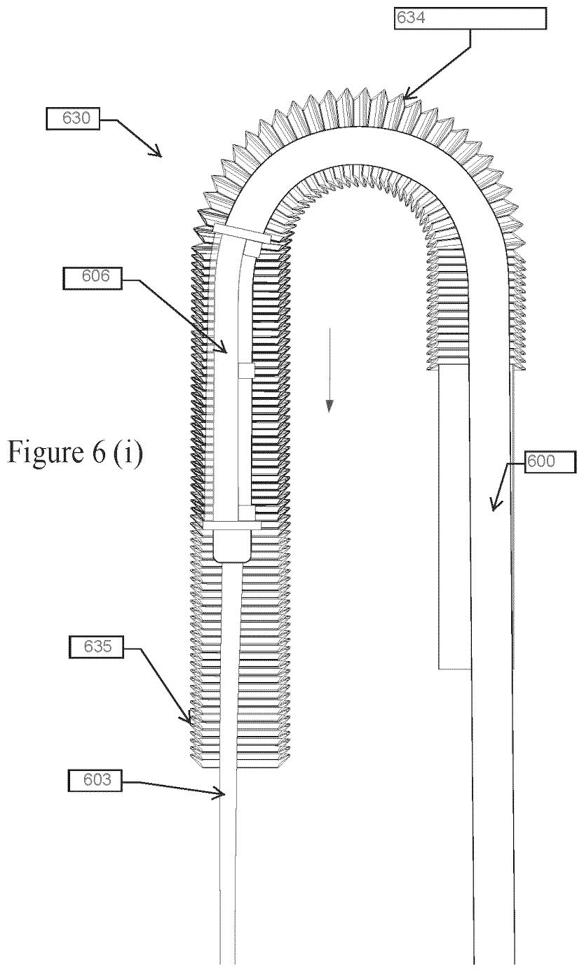

FIG. 6(a)—an exemplary two-dimensional side-view of a double lumen (OTW) shaper 600. The fixed “antenna” 603 and the “bulb” 604 at the tip can be seen.

Exemplary Hydraulic Device Construction (See FIG. 6(a))

The distal end of shaper 600, includes a hydraulic device 606 that optionally comprises an inflatable (elastic) cylindrical tube or membrane 608 (hereafter termed membrane) and is designed to cause CFT 630 to deform (axially and/or laterally). Deformation is provided by latching (e.g., a friction mechanism) radially asymmetrically on to internal wall of CFT 630 while distancing two material points away from each other. When membrane 608 is pressurized, a strain field begins to forms optionally almost exclusively in the surface being designed to yield first (optionally by fabricating areas with thinner thickness to yield first and areas with thicker thickness to yield last; The face of membrane 608 designed to yield first hereafter termed “convex face”, the opposite side hereafter termed “concave face”). This pressure causes membrane 608 to inflate and contact the internal wall of CFT 630, and the operation of curving or otherwise deforming CFT 630 occurs. Shaper 600 optionally includes one or more radio-opaque markers 610 on the concave face of the membrane which can provide imaging feedback as to the location of shaper 600 along CFT 630 and/or as to the curvature of membrane 608 and/or hydraulic device 606.

FIG. 6(b)—Radial cross-section view of shaper micro-catheter 600 with two visible lumens, in accordance with some embodiments of the invention: wire lumen 614 and inflation lumen 616, an inflation orifice 620 supplies pressurized saline to (optionally elastic) membrane 608. An axial cross-sectional view of shaper 600 is also shown to the right of the axial cross-section describing a top cross-sectional view of the above.

FIG. 6(c)—two-dimensional side view (radial) of CFT 630 in an unactuated state, in accordance with some embodiments of the invention. The CFT shown is a particular case of the CFT geometry. Its extension ratio is 1:2.25.

FIG. 6(d)—radial cross-section view of CFT 630.

FIG. 6(e)—radial cross-section view of CFT 630 along with a two-dimensional side-view (radial) of shaper micro-catheter 600 positioned at a proximal end of CFT 630.

FIG. 6(f)—radial cross-section view of CFT 630 along with a two-dimensional side-view (radial) of shaper micro-catheter 600 as it is advanced axially into CFT 630 and positioned at a desired location 634 where shaping of CFT 630 is desired, in accordance with some embodiments of the invention.

FIG. 6(g)—radial cross-section view of CFT 630 along with a two-dimensional side-view (radial) of shaper micro-catheter 600 as it is actuated hydraulically, in accordance with some embodiments of the invention. Upon actuation CFT 630 is optionally curved about the center of elastic membrane 608.

FIG. 6(h)—radial cross-section view of CFT 630 along with a two-dimensional side-view (radial) of shaper micro-catheter 600 as it is hydraulically deflated, in accordance with some embodiments of the invention, optionally as preparation step for another axially advancement along the axis of CFT630.

FIG. 6(i)—radial cross-section view of CFT 630 along with a two-dimensional side-view (radial) of shaper micro-catheter 600, in accordance with some embodiments of the invention, as shaper 600 is advanced further towards (and past) a distal end 635 of CFT 630.

FIG. (Set) 7—a Schematic Layout of the Catheter's Tip in the Context of the Mathematical Model of the CFTs Motion, in Accordance with Some Embodiments of the Invention.

FIG. 7(a), (b)—Top (axial) and section views of a constituent multi-stable cell, respectively, composed of two elastic conical frusta (710, 712).

FIG. 7(c)—Schematic layout of a guide catheter's distal portion (CFT) 700 with a hydraulic device (as part of a shaper micro-catheter) 702 inserted therein. View of the corresponding coordinate system.

FIG. 7(d)—Hydraulic device compartment 702 view in a local coordinate system.

### FIG. (Set) 8—(a) the Theoretical Potential Energy of a Single Elastic Frustum Under Zero Gauge Pressure According to Eq. (11), Alongside the Stable and Unstable Equilibria.

FIGS. 8(b) and (c)—comparison between the theoretical values (dashed curves) and the numerically calculated values (solid black curves) of the potential energy, at the sections described by the dashed lines in panel (a), all in accordance with some embodiments of the invention.

The energetic stability map in panel (a) is obtained from numerical simulation, and shows stable equilibria points in blue and unstable equilibria points in red (colors shown as different shades). In the figure the extension DOF is zeta, and curvature DOF is phi. At zero phi there are two stable axial extension points meaning the cell can be energetically stable when collapsed and when opened. Alternatively, at zero zeta there are two stable curvature points, since the analysis is two-dimensional, the structure can flex to either side, without loss of generality, either side simply means in any azimuthal direction if three dimensions.

FIGS. 12a-12b show the idealized and the realistic deformations of the active frustum along (a) the vertical and (b) the horizontal sections of FIG. 8, in accordance with some embodiments of the invention.

FIGS. 9(a)-(c)—Three projected views describing the dependency of the equilibrium states of a single elastic frustum, on the pressure applied on it directly, in accordance with some embodiments of the invention. Red curves describe the unstable branches whereas blue curves represent stable branches (colors shown as grey shades).

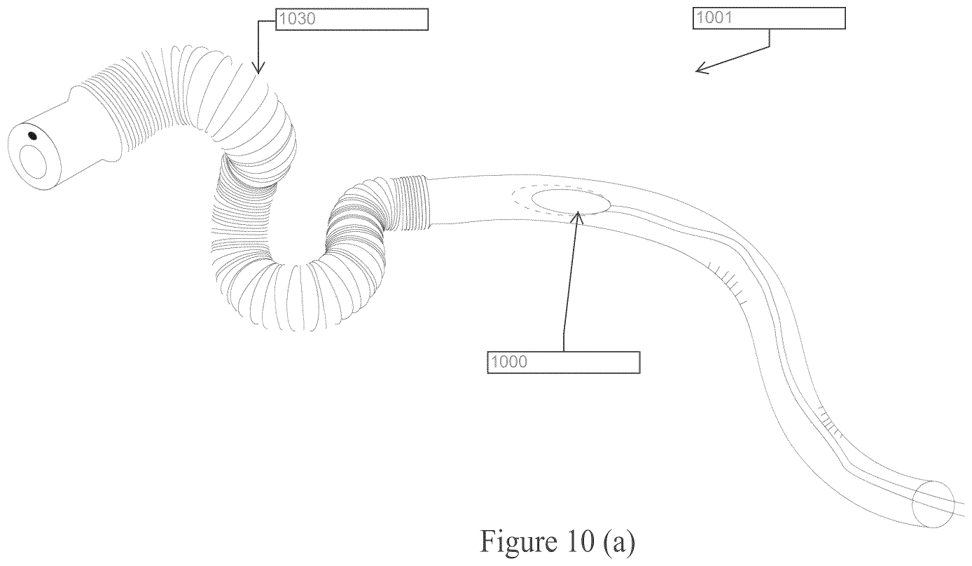

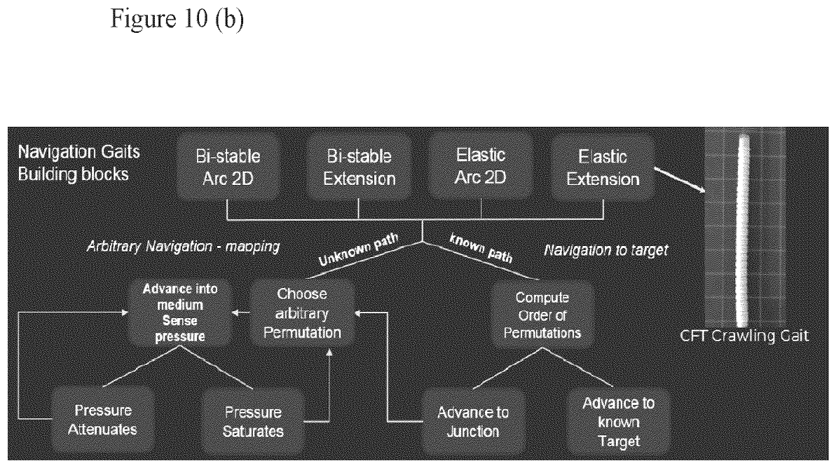

FIG. 10—(a) Schematic layout of a CFT based search and rescue robot 1001 including a CFT body 1030 and an inserted shaper 1000, in accordance with some embodiments of the invention. It is noted that robot 1001 may be tethered (e.g., receiving hydraulic power, computer communication and/or oxygen (e.g., for trapped person's) or untethered (e.g., self-contained with respect to power and optionally receiving instruction and/or power wirelessly. Optionally, robot 1001 includes a processing system for controlling CFT 1030, sensor processing (e.g., if robot 1001 includes a sensor such as an imager) and/or deciding on navigation.

FIG. 10 (b) shows a high level Architecture of a navigation system for use in a disaster zone, in accordance with an exemplary embodiment of the invention.

In some embodiments of the invention, the following method is used:
(a) a human or a computer select a desired target or deformation, for example, based on a desired goal of navigation.
(b) If a target is selected, one or more deformation sets that approximate the target are selected, e.g., using an automated method.
(c) A set of simulations (for example as described herein) are run to find a solution of a set/sequence of inflation pressures and/or shaper positions which provide the desired deformation.
(d) if no suitable set is found, a “failure” indication is optionally generated.

In some embodiments of the invention, one or more simulations are used to generate a set of (optionally maximal) targets that can be achieved. This are shown to a user and/or provided to a computer to define a working set within which to select a next command. For example, such a set may be displayed to a user overlaying an image showing possible targets.

### FIG. (Set) 11—Constrained CFT Sheath/Catheter and OTW (Double Lumen) High Extension Ratio Shaper Micro-Catheter Designed for Hydraulic Transluminal Crawling

The figure set shows a CFT-shaper combination device 1105 which is designed to perform transluminal hydraulic crawling, in accordance with an exemplary embodiment of the invention.

The figure (set) shows a single crawling stroke where a CFT 1100 has advanced distally along a housing lumen 1160 (e.g. artery) axis by pressurization of a shaper 1100. The pulling action of device 1105 is based on extension of CFT 1130, for example, similar to that shown in FIG. 4 of this document, followed by a helical anchoring action of a distal CFT segment 1138 against luminal wall 1160, for example, at locations 1162, 1164 which is finally followed by depressurization action of a hydraulic device 1106 of shaper 1100. The anchoring action helps the depressurization action of the hydraulic device to collapse the CFT cells towards the distal anchor (and not to an otherwise proximal anchor) thereby advancing the catheter distally along the axis.

The collapsing action upon depressurization, which takes place in the current embodiment and in contrast to some other hydraulic mechanisms depicted in this document, is optionally provided by the incorporation of a relatively thick hydraulic device wall (t(x,θ)) and relatively large static radius (R(x, θ)) hydraulic device. The specific choice of hydraulic device properties as per the crawling process is optionally done via convergence of a simulation on the desired crawling function. The process includes a choice of arbitrary wall thickness function at first, and gradual increase of the thickness function during the simulation. As the thickness function increases, actuation pressures increases accordingly. This process may ensure that the hydraulic device wall expands maximally into the CFT cell creases, so that upon depressurization, deflation first forms in the bulk section (centralized along the center axis of the hydraulic device) of the hydraulic compartment. During the first stage of depressurization an optional serrated membrane contour forms a friction force along the CFT thereby collapsing the CFT. In the second stage the friction action will be relaxed, right after the CFT has been collapsed distally. The careful tuning of this process is optionally done by simulation. CFT 1130 itself is optionally coated with an elastic layer applying greater force than resistible by the CFT cells, so that when shaper 1100 is depressurized, the CFT collapses.

FIG. 11(a)—An exemplary two-dimensional side-view of a double lumen (OTW) shaper 1100. An optional guidewire 1102 is seen protruding the tip of the shaper.

FIG. 11(b)—Radial cross-section view of shaper micro-catheter 1100 with two visible lumens in accordance with some embodiments of the invention: a wire lumen 1114 and an inflation lumen 1116. An inflation orifice 1120 optionally supplies pressurized saline to elastic membrane 1108. An axial cross-sectional view of the shaper construction is also viewed to the right of the axial cross-section describing a top cross-sectional view of the above.

FIG. 11(c,d)—Two-dimensional side views (radial) of CFT 1130 before actuation, in accordance with some embodiments of the invention. Locally locked cells 1150 of the CFT 1130 are marked in black along the circumference of each cell, where applicable. Figure (c) shows a side view, and figure (d) complements this side view with an opposite (180 deg) view of CFT 1130. In the example, the locking pattern forms a helix with a centerline which coincides with that of the CFT axis. Other shapes can be provided, including non-uniform and shapes which turn also or instead in the other spiraling direction.

FIG. 11(e)—Radial cross-section view of CFT 1130 prior to actuation. Locked cell locations 1150 are visible where applicable.

FIG. 11(f)—Radial cross-section view of CFT 1130 prior to actuation along with a side view of shaper micro-catheter 1100 positioned at the proximal end of the CFT prior to actuation.

FIG. 11(g)—Following FIG. 11(f) hydraulic device 1106 has been actuated along a straight segment 1134 (prior to reaching the helical pre-shaped segment). CFT 1130 is seen in a radial cross-section view, whereas hydraulic device 1106 is seen in a side view.

FIG. 11(h,i)—View of figure (g) held in position. CFT 1130 is now shown in a confining lumen 1160, such as a vascular lumen.

FIG. 11(j)—Hydraulic device 1106 is now actuated further (e.g., further expansion of membrane 1108) from the point shown in figure (g), so that the helical segment has been reached. Cell by cell CFT 1130 is actuated, each cell responds to the actuation by deforming in the pre-constrained direction dictated by the locking location creating the helix 1150 in the distal segment of CFT 1130. In some embodiments of the invention, CFT 1130 anchors by friction along the confining lumen 1160 (e.g., at points 1162, 1164). CFT 1130 is shown in a side view, overlaid with shaper 1100 and activated and extended hydraulic compartment 1106.

FIG. 11(k)—Seen is the same view of CFT 1130 as figure (j) in its actuated state. Locking (constraining) circumferential locations 1150 are visible in black.

FIG. 11(l)—The same view of figure (k) is shown rotated 180 deg around the longitudinal axis.

FIG. 11(m)—Hydraulic compartment 1106 is now deflated. The deflation causes the collapsing of straight (proximal) cell segment 1134, as the structure is still anchored distally along the helical segment.

FIG. 11(n)—The view (m) is shown in outer side view. The proximal shaft has advanced according to the advancement ratio of straight segment 1130.

FIG. 11(o)—Further depletion of hydraulic chamber 1106 is performed along anchored segment 1138. Shaper 1100 and CFT 1130 are advanced distally. Hydraulic compartment 1106 is retracted and positioned at the distal end of the CFT. In some embodiments of the invention, the distal end of membrane 1108 is designed to collapse after a proximal side. This may be provided, for example, by making the distal side more resilient (e.g., thicker wall) so that it collapses radially before (or faster than) radial collapse of the distal part of membrane 1108 and/or before/faster than axial contraction of the intervening membrane. Optionally or additionally, CFT 1130 may include an elastic layer which is more resilient proximally than distally. Optionally or additionally, inner surface of CFT 1130 may have higher friction distally than proximally.

FIG. 11(p)—The view (o) is shown in outer side view. Catheter 1105 has been advanced distally a distance 1170 along the lumen axis. The sequence (a “stroke”) may be repeated again for a further crawling act.

### Exemplary Integral Device

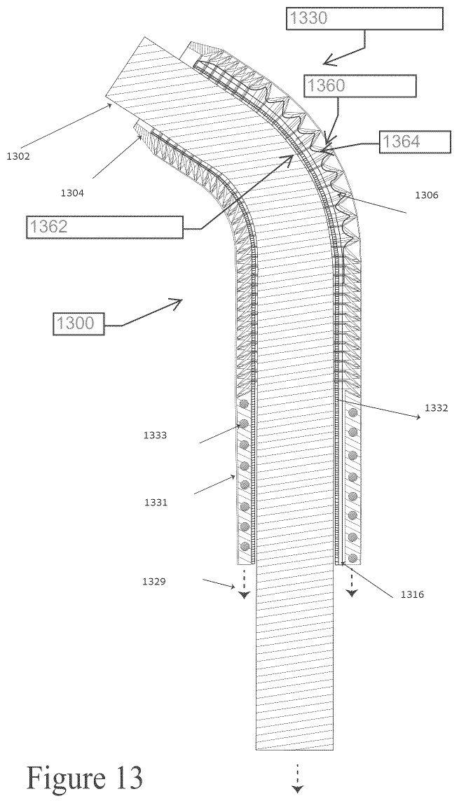

FIG. 13 is a cross-sectional view of an integral device 1300 including a sheath 1330 having CFT like properties and integrated one or more hydraulic compartments 1306 for shaping the sheath, in accordance with some embodiments of the invention. It is to be understood that the features described below may be provided, in part or all, in any of the other embodiments shown herein in the above figures.

In some embodiments of the invention, CFT sheath 1330 includes a set of CFT cells with underlying compartment 1306. When compartment 1306 is pressurized, for example, via an inflation lumen1316, compartment 1306 expands (e.g., it is defined between a CFT wall 1360 and an inner elastic (optionally only or more axially elastic) layer 1362. Layer 1362 may also act as an inner layer for the lumen of CFT 1330.

In this embodiment, friction between compartment 1306 and CFT wall 1360 is not generally needed, do to manufacture as an integral system.

Sheath 1330 is shown as having an optional tip 1304 (optionally soft and/or otherwise atraumatic) and a tool 1302 optionally filling a lumen thereof.

Distally from CFT wall 1360 sheath 1330 extends as a shaft 1329. Optionally, a coil or braid 1333 is provided embedded at least within part of the shaft, for example, to give desired elasticity and pushability properties. Also shown is an optional outer layer 1331. Optionally, layer 1331 is elastic and is, at least in some places, strong enough to snap an underlying deformed CFT cell back to its resting state. Optionally or additionally, layer 1331 serves to constrain deformation of at least some CFT cells at at least some places. Optionally or additionally, elasticity is provided by layer 1362 and/or a different internal layer. In some embodiments of the invention, layer 1362 includes one or more circumferential elements (e.g., thread or wire rings or bands or attachments of layer 1362 to CFT wall 1360) which resist expansion of chamber 1306 into a lumen of CFT 1330.

Also noticeable in the figure is an optional design whereby compartment 1306 includes also an elastic layer 1364 beneath wall 1360, which layer 1364 is shown expanding into creases defined by wall 1330. A potential benefit is that this allows chamber 1306 to be sealed and provided as a unit, rather than sealed to CFT 1330. Optionally or additionally, it allows CFT wall 1360 to have one or more apertures formed therein.

Device 1300 of FIG. 13 is optionally used for medical access in the heart and tool1 302 can be, for example, an ablation tool. In some embodiments of the invention, device 1300 extends to a handheld (or other) hub outside the body which may include, for example, a pressure source and/or user controls for selecting section of CFT 1330 to deform and/or an amount of deformation.

In some embodiments of the invention, a plurality of lumens 1316 are provided, one for each section to be expanded, for example, for a pre-set bend section, for a universal bend section and/or for axial extension section.

A potential advantage of an integral hydraulic compartment is obviating the need to align a shaper axially and/or rotationally with the CFT.

A potential advantage of an integral device is allowing a simpler mode of operation as the shaper need not be retracted when a tool is to be used.

A potential advantage of an integral device is allowing elastic deformation which using a tool.

Specific exemplary use cases based on the some embodiments of the invention are now described.

### Exemplary Peripheral Vasculature Catheterization

The mechanism, methods and designs discussed so far have laid the basis for a steerable catheter device to be used in conjunction with a micro-catheter and optionally a guidewire, which may be provided in accordance with some embodiments of the invention. In a radiological setting, a micro-catheter can have two functions as shown in the figures. First the micro-catheter can provide distal anchoring and support for further navigation, placement of the guidewire and transfer of treatment to the target (e.g. embolization)—the conventional function of a micro-catheter in peripheral vasculature catheterization. Secondly, the microcatheter functions as a CFT shaping device when taking the functional pressure control signals that strains the hydraulic device/compartment at its tip, in turn actuating and steering the CFT. The functional role of the guidewire is optionally retrained in this design, making it suitable for steerable peripheral intervention settings. In some embodiments of the invention, the target size of the CFT for this use-case is 5-6 Fr while the double lumen micro-catheter target size is 3-4 Fr. A variety of guidewires, as large as 0.035″ can fit the design. The suggested method could provide precision three-dimensional shaping/steering of the CFT catheter for further placement of the micro-catheter and/or transfer of treatment to the target. Extension capabilities could also contribute as a navigation method and where torque transfer becomes challenging. Note that when using a type 2 hydraulic device (see table 1) in conjunction with an unconstrained CFT catheter/sheath, steering is optionally achieved without the need for torque transfer at all. In some embodiments of the invention, steering comprises selecting a direction to bend the sheath and applying that bending, rather than rotating the sheath and applying a pre-set bending direction.

Even in embodiments where an inner shaper needs to be rotated, this does not generally require a same amount of torque transfer and/or precision as when rotating the sheath itself, especially when the shaper is deflated and takes up a smaller diameter.

In some embodiments of the invention, the shape has three circumferential compartments, each one extending less than 360 degrees, for example, each one extending between 30 and 120 degrees. This allows a universal bending mechanism to be provided. Selective inflation of one or two compartments causes the shaper to expand and the surrounding CFT to deform along a direction defined by the selection and degree of inflation of these compartments.

In some embodiments of the invention, a CFT section is shortened using a two balloon system—two balloons which expand to engage the CFT at different parts and spaced apart by a section which can be shortened and which does not engage the walls. After inflation, the section is shortened, thereby shortening the CFT. For example, shortening may be provided by each balloon being mounted to a different pull wire and then pulling on the pull wire of the more distal balloon. This method can also be used with a single balloon shaper, whereby the balloon is inflated to engage the CFT and then the shaper is pulled back when holding the CFT in place.

In addition, using a constrained CFT design (e.g., CFT types 3 and 6) along with a symmetric shaping device (e.g. type 1, 3, 4) may also reduce a need for torque transfer dependent steering. Finally, in the peripheral vascular setting a potentially significant advantage of the CFT-Shaper combination device is control of local curvature of the distal shaft which can minimize arterial support and anchoring. For example, in convention radiology, in order to turn into an ostium, say from the aorta, the physician typically uses a specific catheter design that anchors against the aorta and pivots into the ostium. A CFT catheter can be curved into the ostium using pivoting of a proximal segment of the CFT (shaft) relative to the distal segment being positioned into the ostium and optionally without anchoring or leaning against the aorta and also without any sliding movement against to the aortic wall.

### Exemplary Manual Interface Steerable Catheter

In some embodiments of the invention, the combination of a CFT catheter and a hydraulic shaping catheter, for example as described above and herein and/or in FIGS. 1-4, together form a steerable catheter device. The device can be actuated manually by the equivalent of a PTCA balloon inflation device optionally integrated into the hub of the CFT catheter and/or shaper. The hub can potentially facilitate the axial and rotational alignment between the shaping device and the CFT device. When single membranic compartment shaping devices are used (see table 1), torqueing of the shaper (micro) catheter is optionally used to control the plane of the local curve. In some embodiments of the invention, the closed environment of the CFT catheter lumen optionally provides the basis for precision rotational alignment between the two catheters (CFT and shaping catheter). The design of the hub of the CFT catheter optionally includes dial type controls for the control of the rotational alignment of the shaper and CFT, and/or control of the saline pressure in the hydraulic device. The hub will optionally also include a locking mechanism for the positioning and retraction of the shaper catheter.

### Exemplary Robotic Interface Steerable Vascular Catheter

Robotic vascular catheterization platforms are gaining growing attention these days. Some of their potential advantages are low radiation exposures to the staff and patient (with a radiation protected control interface), the ability to perform procedures remotely (even over 5g wireless connections), perhaps even with no highly trained staff on location, and overall improved clinical outcomes. Currently, robotic catheterization platforms can be classified into two main market segments. Non-steerable technologies, based on conventional manual catheter disposables (e.g., as by Siemens Corindus and Robocath), and steerable technologies, dispersed in a variety of vascular applications, each based on a dedicated catheter design which is compatible with the robotic interface. The first segment has been the focus of recent advances in this field with relatively low procedural cost and a low cost of disposable catheters per procedure. However, this first segment suffers from low steerability or the equivalent of non-steerablity in the manual operation segment and thus is limited in scope; it typically excludes: complex lesion PCI, cardio catheterization, peripheral vascular catheterization and more. The second segment suffers from high procedural costs and a high cost of disposables, sometimes reaching several thousand dollars for a single catheter. In some embodiments of the invention, there is potentially provided a precision robotic interface steering at a low cost per procedure while maintaining catheter modularity and cath-lab workflow as in the manual segment.

The combination of the CFT and hydraulic shaping catheter, as described above and herein and for example in FIGS. 1-4, provide the basis for robotically controlled steering. A potential advantage of this method of control is that the catheters, namely the CFT catheter and the shaping catheter, may require only slight modification, if any, of the manufacturing processes of conventional manual catheters (vascular), e.g., tip modification of conventional guide catheters (to use as a CFT device) and balloon modification of a PTCA catheter (to use as a shaping catheter). Once these are achieved, the robotic interface can provide precision actuation and steering of relatively cheap disposable catheters that together form a steerable catheter device with multiple degrees of freedom. Another potential advantage when considering the robotic platform is that the CFT and shaper combination device can be actuated by a dedicated control algorithm (e.g., based on the solution of the CFT governing equations described above) in which position feedback of the catheter's tip is achieved via a combination of cath-lab imaging resources and a unique dynamic pressure reading. For example, a table may be provided linking pressure and shape, which table is optionally associated with the shape and strain field of the hydraulic device. Optionally or additionally, one or more rules are provided, for example, a high rise in pressure may indicate that the catheter is stuck. In another example, the internal pressure of the hydraulic device may change not only as a function of actuation, but also as a function of stresses exerted by the artery wall. Continuous reading of hydraulic device pressure (p in the potential energy of the frustum, denoted Vn,k above) can enable to generate a more correct position and arc shape of the CFT by a feedback mechanism and control algorithm. In some embodiments of the invention, the robotic controls of the CFT-shaper combination device can include one or more of axial position of the CFT catheter, torque and axial position of the shaper catheter (in shaper types 2 and 4 torque is not necessary), pressure control of the membrane compartments of the hydraulic device (at the tip of the shaper catheter) and/or torque and axial position of the guidewire where applicable.

In some embodiments of the invention, a model is built which matches sensing pressure signals with actual deformations and/or forces applied on a CFT. For example, a data set may be created of pressures and images (e.g., X-ray) or built using a simulation. When an actual signal is sensed, the model is used as a look up to determine the deformation or pressure. Such a model may be implemented, for example, as a lookup table, a neural network or a machine-learning model, (e.g., a weighted parameter set model), among other manners. Links in both direction are possible-what pressures, when applied cause what deformation and vice versa, what pressure signal when sensed indicates what force. Also, an analytic solution can be provided using equations which model the connection between geometry, deformation, pressure and transients.

### Exemplary Transseptal Access System

In some embodiments of the invention, a structural heart access/placement sheath is provided. The sheath can be actuated either manually or robotically. For example, using a combination of type 2 and 4 (table 1) hydraulic devices, actuation can be achieved without the need to apply rotational torque to the sheath and/or the in-lumen hydraulic device. Optionally, local curvature of the CFT is controlled keeping its structure stable both under actuation and after actuation. The sheath can also be optionally locked into place via a constrained CFT mechanism (e.g., where necessary) to provide further support for delivery of transcatheter devices, once the shaping mechanism has been retracted. A large delivery lumen can be cleared once the hydraulic device has been retracted providing a good inner to outer dimeter ratio of the sheath, for example, above 70%, above 80%, above 85%, above 90% or smaller or intermediate percentages. The extension capabilities of the sheath combined with curvature control can provide good co-axial alignment capabilities for mitral valve delivery and/or treatment (e.g., to align a sheath axis with a valve axis). For example, when considering an equivalent CFT mitral delivery device, in direct comparison with the mitral-clip system for example, one or more of the following potential advantages are provided: A single delivery CFT sheath could provide all the necessary navigation and placement capabilities, potentially, in a simply operated, robotically controlled (or single human), device as oppose to a manually controlled device requiring 2 expert physicians and several more cath-lab technicians. Extension capabilities of the CFT can potentially provide delicate and precise controlled corrections instead of or in addition to the traditional push-pull movements of the physician. In some cases, septal puncture is performed and the steering of the distal, left atrial sheath is quite limited in the mitral setting. The ability to locally control the distal sheath, via the shaping device post interatrial puncture can provide unique and precise delivery capabilities. The retractable hydraulic device can provide a relatively large available delivery lumen with respect to the outer diameter, perhaps opening further markets for left atrial procedures, such as children. Another potential benefit is that once a part of the CFT is shaped, it can remain stably in that shaped configuration while a next section is shaped. This allows sequential rather than parallel manipulation which may be more precise and/or require less expertise.

### Exemplary Cardiac Uses

In some embodiments of the invention, a heart access/placement sheath is provided. Optionally, the sheath is used for delivering cardiac tools, for example, cardiac ablation tools, optionally standard such tools, optionally tools without articulation ability (e.g., so any articulation needed may be provided by the sheath).

In one example, the sheath is according to FIG. 4, though other designs, such as in the other figures showing sheaths or otherwise described herein, can be used as well.

In some embodiments of the invention, the sheath is used to access complex anatomical structures, for example, structures having a complex path thereto. One example of a complex path includes passage through a cardiac valve (in all, possibly two valves), after which further extension or curves are required. It si noted that in these delicate structures, pushing—or advancement from the hub can be challenging or cause adverse events. Another example of a complex path is a path which includes an S shaped curve, or an S shaped curve after which further navigation is required (such as to the Right Ventricular outflow tract (RVOT)).

For example, a sheath can be configured by controlling several segments of the distal shaft (not just a simple curve as in conventional steerable catheter designs) as shown in the above described figures drawings (and optionally including the use of presets such as in FIG. 4). Such configuration may allow the sheath to be extended even to distal regions of the LVOT or RVOT (left and right ventricle outflow tract, respectively) without compromising the heart's delicate structures. After reaching these areas further distal manipulation may still be available and can be leveraged to precisely navigate a therapeutic tool through the sheath in these areas. Sheath access to these areas could be used to guide therapeutic tools to these locations, for example, for ablation, for delivery of implants such as a pacemaker or mitral clip, for performing valvuloplasty and/or for cardiac electro-anatomical mapping.

A potential benefit of using a controllable sheath as described herein is the stability of the sheath once shaped, which can in turn provide stability of access to a target, potentially without leaning on that target, even for targets where currently stable access is difficult or impossible to access or supply a stable sheath thereto. Once at the target area, as described herein, the tip of the guide sheath can be moved, to help access nearby locations.

In a particular implementation, a sheath device comprises a multi-stability guide sheath with outer elastic layer which is optionally configured as a soft sheath (without any braided or coil metal reinforcements of the shaft). In one exemplary use, the sheath device is advanced using hydraulic shaping to complex anatomical areas of the heart potentially without endangering and/or damaging the delicate internal structures of the human heart and/or without deforming the heart or causing an unintended arrhythmia. Once in place, the sheath device may be stiffened (e.g., using the described-herein hydraulic shaping mechanism) to provide support for passing tools and treating the heart.

In one example of use, axial advancing of the tip is used to position the sheath, after it is correctly placed. For example, the sheath may be navigated and curved, as needed, and an actual length set after navigation and curving si complete. In contrast, standard catheters can only be advanced by pushing, so the location of any curve depends on pressure against body surfaces, and sliding of the catheter at the bent location (applying friction and force to anatomy) is needed in order to complete tip advancement. The therapeutic tool (e.g., ablation catheter may be positioned in the sheath during navigation or provided after. In a system with a removable hydraulic shaper, it will typically be provided after, to reduce overall diameter.

A potential advantage of tip bending as described herein is that any applied forces are applied at the tip of the catheter and do not need to be mechanically (by pushing or pulling) be conveyed along the catheter/sheath. Also as the sheath can be stiff other than at the end, such bending does not interfere with the layout of other parts of the catheter/sheath. Conversely, the actually layout of the catheter/sheath is not expected to affect the bending, while in a typical pull-wire based catheter, friction and forces and bending of the catheter/sheath along its length may affect the quality and/or quantity of the movement. Potentially this allows for more precise, predictable and/or stable movements.

Another potential advantage of the design described herein is that the catheter or sheath lack coupled tip-shaft movements and tool-sheath coupled movements as may be present in a standard sheath/catheter design. In some embodiments of the invention, hydraulic deflection and extension replace pull-wire tension (or coaxial shaft positioning) and push-pull movements (typically applied via a handle). This has a potential advantage of reducing dependency of positioning, navigation and stability on operator hand movements and dexterity.

### Exemplary Colonoscopic Access System

In some embodiments of the invention, the basis for a colon diagnosis and/or treatment scope, e.g., a CFT colonoscope, is provided. A potential advantage in this use case revolves around significantly lower support and strain against the colon, which are considered to be known drawbacks to patient outcomes in conventional colonoscopies and potential causes of tearing or perforation or other colonic trauma. Optionally or additionally, what is improved is fine control of the tip location, a problem exacerbated in standard colonoscopies by the elasticity and movability of the colon. Tip resistance can be reduced due to the local control of shaft curvature, minimizing lateral movement along the colon, especially around the sigmoid loop. In addition, extension capabilities of the CFT scope can provide the potential capability to advance the distal shaft gradually, without the need to push the proximal shaft at all, thus potentially reaching a target safely and minimizing perforations. In some embodiments of the invention, local control of the stiffness of the scope is provided using the in-lumen hydraulics. In some embodiments of the invention, a relatively (e.g., to what can usually be used in the heart) large and permanent hydraulic device can be used. Optionally, these are provided such that all possible controls along the CFT colonoscope shaft are available to the physician. In the colonoscopy setting, a robotics interface potentially garners advantages, among them hydraulic shaper compartment feedback which can also signify to the algorithm if the tip is progressing smoothly without lateral forces or if lateral forces rise and corrections must be made.

In some embodiments of the invention, when there is more lumen space available, one can employ a 3 compartment hydraulic device with omni-directionality (e.g., type 2 shaper table 1). Feedback is optionally provided by reading the proximal (hub) pressure of a specific lumen. Generally, in these cases, the control signal is saline flux and the controlled variable is saline pressure.

### Exemplary Search and Rescue Robot

In the event of large crises, a primary task of the fire and rescue services is the search for human survivors on an incident site. This is a complex and dangerous task, which—too often—leads to loss of lives among the human crisis managers themselves. This section of the document introduces unmanned search and rescue robot/device technology that is based on the mechanism discussed so far regarding the steerable catheter device, but with adaptations in scale, design and/or methods of operation which may make it more suitable for the debris environment in which the robot/device will be used. Such robot/device, equipped with currently available miniature sensing technology such as one or more of high-definition video cameras, thermal cameras, 2D and 3D laser range finders, spectrometers, chemical sensors, microphones and/or other sensors, can allow for locating casualties trapped underground and/or perform precise and/or relatively fast cartography of the searched area. For other purposes such as pinpointing potential dangers, sensors such as for measuring chemical, biological, and radiological contamination can be used. A potential advantage of using an unmanned robot/device is that the human operators can be kept remote and safe.

In some embodiments of the invention, the search and rescue robot/device is mostly designed to serve as a tool by the hands of human search and rescue workers that are in a mission of “tunneling through debris”. In such a mission the rescue team try to pave a way into a pile of debris (e.g. of collapsed building) looking for signs of life and search after cavities with trapped air, where people could survive. The search and rescue robot/device is adapted to that kind of environment and is capable of navigating in an uncertain medium, progressing and bending in accordance to a three dimensional maze created by the debris, sometimes through narrow branch out and tortuous tunnels and sometimes in an open space without supports to lean on.

It is noted that the described (optionally) soft robotic mechanism is optionally designed to penetrate into the debris. Optionally, it's the head (distal tip) of the robotic mechanism leads the search, progressing into the debris but the tail (proximal end) is always left outside the debris, so a passage is maintained. Once located, it is possible to supply aid (e.g. oxygen, water, food, communication means) to the trapped survivor through the lumen of the robot itself.

A potential advantage of the design is that such a robot/device can be steered to progress/grow in any direction along its axis and be bent to any required 3D curve. In contrast to some snake-like mechanical robots and growing soft-robots, some embodiments of the invention can recover its tracks at any point along the path and alter the navigation route.

As with some embodiments of the steerable catheter device, the search and rescue robot/device is optionally actuated manually by hydraulically pressurizing an asymmetric and/or compliant balloon/membrane inside the CFT lumen. (e.g., See FIG. 11) Optionally, the CFT will include a hub that provides axial and/or rotational alignment between the shaping device and the CFT can work in the same way as in the steerable catheter device.

In some embodiments of the invention, a guidewire is used in the steering of the search and rescue robot/device. Wires are typically characterized by their pushability, steerability, and torque. Pushability is the amount of force needed to advance the wire. Steerability is the ability and responsiveness of the wire tip to navigate vessels. Torque is the response of the wire to turning by the operator e.g. when navigating in debris. Guide wires typically come in two basic configurations: Solid steel or nitinol core wires and solid core wire wrapped in a smaller wire coil or braid. Coiled or braided wires typically offer a large amount of flexibility, pushability and kink resistance. Guide wires usually have a floppy tip and a stiff body to enable easy tip navigation, with good pushability offered by the stiffer section of the wire. Some wires are coated with a polymer, such as silicone or polytetrafluoroethylene (PTFE), to increase lubricity. The tips of the wires come in various configurations, including a “J” curve, a variety of angles or straight tips to help navigate various 3D obstructions. When considering guidewire diameter for search and rescue application, it is optionally be scaled up from sizes used in medical application, for example to a diameter of approximately around 10 mm, but consideration of shape, diameter, length, and stiffness are optionally customized dependent on mission and application.

One way a guidewire can be used in navigation is by introducing it into the CFT lumen to guide the CFT above complex wreckage/debris terrain or pass obstacles when a curved guidewire is used. The wire is advanced and later the CFT is along it, using the wire as a rail, and then again the wire is advanced and so on. At any time the shaper can be inserted instead of (or over or to the side of) the wire, into the CFT lumen to create a desired bending or extension. As noted herein, the CFT can be stable in maintaining its curvature, at least in some parts thereof, e.g. after transition of bi-stable state, to resist the wire or shaper forces when introduced through it, and in that manner the CFT tip is directed toward the next destination of navigation, to which the wire is directed in the following step. In some embodiments, at least part of the CFT has an elastic element or layer associated with it, to undo the deformation of the CFT by the shaper. When navigating in open space, the guidewire can stand only limited length without bending to the ground under gravity forces, in that case the CFT is optionally guided with the wire on the ground, until a point is reached where the CFT is required to be bend upward. Then the shaper is introduced to desired location along the CFT, and actuated to create the required bending of the CFT.

In some embodiments of the invention, a CFT (in this or any other embodiment) may include some parts which are stable and some which elastically recoil. For example, some parts of the CFT may be provided with an elastic layer and some not, or with a thinner and/or otherwise less resilient layer.

Another way a guidewire can be used in navigation is that it is introduced into an assigned lumen in the shaper, to guide the shaper through tortuous curves in the CFT and over the wavy surface which may characterize the inner surface CFT. In some embodiments, the inner surface is coated to make it smoother, as may be the medical tool inner surface.

The lumen of the CFT is optionally larger in the search and rescue application in comparison to the steerable catheter device. Its diameter can be of the order of a few centimeters (normally 2-5 centimeters, but it can be larger, for example, 5-10 cm or 10-20 cm or more). For such lumen diameter it may be advantageous to design a mechanical robot controller to actuate the CFT. Such robot controller will either bend the CFT, or extend it, or both. Such robotic controller may translate commands (e.g., given by GUI or a joystick or knobs) into inflation/deflation/advance/retreat commands for a shaper. Optionally or additionally, no handheld controller is provided, rather, a user may indicate a target location or a general command, such as “explore” and the robotic interface translates such commands into actions by the shaper and the CFT, for example using planning systems as known in the art.

An option for the actuation of the search and rescue robot/device is by robotic interface. The CFT shaper and guidewire retain their original form, and a suited control system is used for actuating them. From a mechanical prospective the actuation is in the same way as a human would, e.g., the control system controls electronic gear that mechanically shifts the CFT forward and backward and set its axial position. Similarly, electronic gear attached to the shaper and/or guidewire, can apply and/or sense torque and/or axial friction force to determine axial and rotational position of the shaper and/or guidewire. Other layout sensing methods may be used. In case of hydraulic shaper, the pressure in the balloon/membrane compartments is optionally controlled, and results in controlled bending and/or extension of the shaper. In case that mechanical robot is actuating the CFT the mechanism is optionally fully electrically controlled.

The actuation by robotic interface as just mentioned can be done by human operating a joystick, with different action to control each degree of freedom (e.g. pull the stick once for driving the CFT backward in axial position 1 mm, press a button and take the joystick left to rotate by 5 degrees the shaper counterclockwise, etc.). One way for actuating the robotic interface is by a dedicated control algorithm that receives as input the position of the catheter's tip within a coordinate system (or on an image) optionally deduced by 3D sensing means, such as 3D laser range sensors or stereo imaging.

FIG. 10(a) shows an illustrative view of the CFT based search and rescue robot 1001 as well as a chart 10(b) showing a navigational algorithm of the suggested search and rescue robot, based on hydraulic shaping, in accordance with some embodiments of the invention, including navigational capabilities and gaits.

Such control methods may also be used in medical settings. For example, a dedicated control algorithm (based on the solution of the CFT governing equations such as described herein, for example) in which position feedback of the catheter's tip is achieved via a combination of cath-lab imaging resources and a unique dynamic pressure reading associated with the shape and strain field of the hydraulic device. For example, the internal pressure of the hydraulic device may change not only as a function of actuation, but also as a function of stresses exerted by the artery wall. Continuous reading of hydraulic device pressure p in the potential energy of the frustum (denoted V_{n,k} in the section entitled “An exemplary conical frusta tip” above), can enable to generate the true position and arc shape of the CFT by a feedback mechanism and control algorithm. These methods may also be used for non-medical settings.

Exemplary Specific Designs Stemming from the Disclosure and Having Specific Interventional Uses

The following is a list of designs which are based on the technology described herein and which correspond to specific interventional use cases and which may be provided in accordance with some embodiments of the invention. Sizes and forces and/or other design elements of the general mechanism described above are optionally adapted to match the below uses, especially, but not only, using features known in the art:
1. Steerable guidesheath (with or without the use of a guide-wire). In particular it may be useful to have uncoupling between setting of bending (location and/or angle) and setting of axial extension (location and/or amount).
2. Steerable catheter (A guidewire leads inside the lumen, shaper runs over the wire)
3. Steerable ablation catheter (actuation of the CFT by a pressurized integral lumen, non-retractable device)
4. Steerable guidewire (actuating the CFT by push-wire)
5. Steerable colonoscope
6. Steerable coronary delivery sheath (optionally similar to item 1, but one or more optional specific features that match needs in the coronary system-local permutations (and predefined (e.g., during manufacture) bend locations, direction, constrictions) of CFT, for example to match known geometries, providing support without anchoring, navigating a 3D space using arbitrary 3D curves and extensions which do not rely on proximal push or twist)
7. Decoupling shaft rigidity from push-ability and/or torque-ability of the catheter (e.g., the shaper and CFT are aligned, twist of the shaper changes the directionality of the curve without the need to twist the CFT sheath).
8. Steerable transseptal catheter
9. Steerable snake robot, tethered or untethered, for searching in chaotic environment such as debris
10. Dynamic shaft extensions, which allow, for example, to select a direction stably and then advance the tip of the catheter to the desired location
11. Crawling shaper
12. Providing open loop precision-angle of bending is a direct function of the pressure, due to stability and constraints provided by CFT geometry
13. Interventional radiological setting—the shaper MC functions as a conventional MC (microcatheter), while the CFT (e.g., 5 F in diameter) provides precision steering
14. Transeptal delivery—transfer higher torque—independent of shaft and steering
15. Some example intrabody sizes: 6 Fr OD catheter—MC—3-4 Fr, wire 0.018

### LIST OF SYMBOLS

lₜ-conical frusta tip unactuated axial length
lᵣ-axial extension ratio of the tip
rᵢ-inner frustum radius
rₒ-outer frustum radius
E-Young's modulus
h-CFT wall thickness

### LIST OF ABBREVIATIONS

CFT—Conical Frusta Tip

### General

It is expected that during the life of a patent maturing from this application many relevant multi-stable designs will be developed; the scope of the term multi-stable is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-70. (canceled)

71. A multi-stability guide sheath, comprising:
a body, comprising:
a lumen; and
a bendable section enclosing a part of said lumen and having an axial extent and defining along a side thereof a sequence of a plurality of axially spaced apart deformation points, each point defined by at least one crease,
wherein each deformation point is characterized by having multiple locally stable deformation states.

72. A multi-stability guide sheath according to claim 71, wherein deformation at said points comprises buckling at said crease.

73. A multi-stability guide sheath according to claim 71, wherein said plurality of axially spaced apart deformation points along said bendable section each define at least two minima of resistance to bending.

74. A multi-stability guide sheath according to claim 73, wherein said creases define a CFT-like structure of a wall thereof and wherein said CFT-like structure is defined, at said points, by pairs of radially converging and diverging surfaces of said wall.

75. A multi-stability guide sheath according to claim 74, wherein said CFT-like structure is defined by a diverging frustum and a converging frustum meeting at a crease.

76. A multi-stability guide sheath according to claim 74, wherein said CFT-like structure is not circumferentially symmetric.

77. A multi-stability guide sheath according to claim 71, comprising one or more circumferentially extending wire.

78. A multi-stability guide sheath according to claim 71, wherein said body is elastic and/or comprises an elastic layer.

79. A multi-stability guide sheath according to claim 71, comprising at least one integral fluidic expandable chamber which is configured to deform said bendable section, when expanded.

80. A multi-stability guide sheath according to claim 79, wherein said at least one integral fluidic expandable chamber comprises a plurality of said chambers for selective expansion for providing bending and/or extension of said bendable section and wherein said plurality of chambers are underlying said bendable section.

81. A multi-stability guide sheath according to claim 79, wherein said fluid is a gas or a liquid.

82. A multi-stability guide sheath according to claim 79, wherein said chamber is defined by a membrane between said chamber and a wall of said body.

83. A multi-stability guide sheath according to claim 82, wherein said membrane is constrained from expanding into said lumen.

84. A multi-stability guide sheath according to claim 79, having a single central inflation lumen.

85. A system comprising a multi-stability guide sheath according to claim 79 and circuitry for calculating a position of at least a part of said sheath or resistance of surrounding structure based on a pressure in said chamber.

86. A multi-stability guide sheath according to claim 71, comprising at least one removable shaper sized to fit in said lumen and shape said bendable section.

87. A multi-stability guide sheath according to claim 86, wherein said shaper has an outer surface configured to frictionally and/or geometrically engage an inner wall of said lumen.

88. A multi-stability guide sheath according to claim 86, wherein said shaper has a predefined axially asymmetric shape.

89. A multi-stability guide sheath according to claim 71, wherein said bendable section has at least one restrictor which non-uniformly limits deformation of said bendable section.

90. A multi-stability guide sheath according to claim 71, wherein said sheath is configured to be bent to create a compound curve in said bendable section.

91. A multi-stability guide sheath according to claim 71, wherein a first deformation at said plurality of creases defines a different axial extension of said guide sheath than a second deformation at said plurality of creases.

92. A multi-stability guide sheath according to claim 71, wherein said creases are arranged to allow a range of bending of said bendable section of at least 60 degrees of bending between one position and a second position, the bending being measured relative to a longitudinal axis of said sheath.

93. A multi-stability guide sheath according to claim 71, wherein said creases are arranged to allow said body to bend with a proximal curve, an axial extendible section and a more distal curve, wherein one or both of said curves is at least 180 degrees.

94. A multi-stability guide sheath according to claim 71, wherein said guide sheath is an intrabody guide sheath and wherein said lumen has a cross-sectional area of at least 80% of an area defined by an outer cross-section of said guide.

95. A multi-stability guide sheath according to claim 94, wherein said guide sheath is a cardiac guide sheath.
